(19) United States
(12) Patent Application Publication
(10) Pub. No.: US 2017/0202840 A1
(43) Pub. Date: Jul. 20, 2017

(54) TREATMENT OF PANCREATIC CANCER WITH LIPOSOMAL IRINOTECAN

(71) Applicant: MERRIMACK PHARMACEUTICALS, INC., Cambridge, MA (US)

(72) Inventors: Eliel Bayever, New York, NY (US); Navreet Dhindsa, Boston, MA (US); Jonathan Basil Fitzgerald, Arlington, MA (US); Peter Laivins, Scituate, MA (US); Victor Moyo, Ringoes, NJ (US); Clet Niyikiza, Gulph Mills, PA (US); Jaeyeon Kim, Lexington, MA (US)

(21) Appl. No.: 15/403,441

(22) Filed: Jan. 11, 2017

Related U.S. Application Data
(60) Provisional application No. 62/278,751, filed on Jan. 14, 2016.
(60) Provisional application No. 62/281,458, filed on Jan. 21, 2016.
(60) Provisional application No. 62/328,830, filed on Apr. 28, 2016.
(60) Provisional application No. 62/341,223, filed on May 25, 2016.
(60) Provisional application No. 62/355,640, filed on Jun. 28, 2016.

(51) Int. Cl. A61K 31/519; A61K 31/4745; A61K 31/513; A61K 9/00

(52) U.S. Cl. CPC A61K 31/519; A61K 9/0019; A61K 31/4745; A61K 31/513

(57) ABSTRACT

A liposome-encapsulated formulation of irinotecan can be used for the treatment of pancreatic cancer in combination with 5-fluorouracil and leucovorin, including treatment of patients diagnosed with metastatic adenocarcinoma of the pancreas who have been previously treated with gemcitabine.

### RELATED APPLICATIONS

This patent application claims priority to each of the following pending U.S. provisional patent applications, each incorporated herein by reference in their entirety: 62/278,751 (filed Jan. 14, 2016), 62/281,458 (filed Jan. 21, 2016), 62/328,830 (filed Apr. 28, 2016), 62/341,223 (filed May 25, 2016) and 62/355,640 (filed Jun. 28, 2016).

### TECHNICAL FIELD

The present disclosure relates to the treatment of pancreatic cancer and the use of the topoisomerase 1 inhibitor irinotecan liposome injection for intravenous infusion, including use of irinotecan liposome injection for the treatment of metastatic adenocarcinoma of the pancreas in combination with fluorouracil and leucovorin in patients who have been previously treated with gemcitabine-based therapy.

### BACKGROUND

Pancreatic cancer is the fourth leading cause of cancer death in the United States and in Europe. Despite improvements in cancer treatments, there remains a critical need to further improve therapies to prolong patients' lives while maintaining quality of life, particularly in the case of advanced cancers such as pancreatic cancers that often are, or become, resistant to current therapeutic modalities. Incidence of pancreatic cancer has markedly increased during the past several decades. It now ranks as the fourth leading cause of cancer death in the United States. Pancreatic cancer's high mortality rate is due to a dearth of effective therapies and a complete absence of reliably durable therapies. Because of the location of the pancreas, pancreatic cancer is typically not diagnosed until a tumor has become large enough to produce systemic symptoms. This, coupled with the absence of good screening tools and a limited understanding of risk factors, results in patients usually having advanced disease, often advanced metastatic disease, at the time of diagnosis. Metastatic pancreatic cancer has a dismal prognosis and is almost uniformly fatal, with an overall survival rate of less than 4% at 5 years.

Chemotherapy with one or more of 5-fluorouracil (5-FU) and gemcitabine has been shown to prolong survival in pancreatic cancer. Combination therapies including folinic acid (leucovorin or levoleucovorin), 5-fluorouracil, and irinotecan (FOLFIRI), folinic acid, 5-fluorouracil, irinotecan and oxaliplatin (FOLFIRINOX), or, less commonly, a combination of folinic acid, 5-fluorouracil, and oxaliplatin (FOLFOX) are also used to treat some pancreatic cancers. Irinotecan is 7-ethyl-10-[4-(1-piperidino)-1-piperidino] carbonyloxycampothecin, IUPAC name (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate. Irinotecan is a member of the topoisomerase I inhibitor class of drugs and is a semi-synthetic and water soluble analog of the naturally-occurring alkaloid, camptothecin. Also known as CPT-11, irinotecan is currently marketed as CAMPTOSAR® (irinotecan hydrochloride injection), provided as an irinotecan hydrochloride trihydrate salt. CAMPTOSAR® is approved as a component of a therapeutic combination with 5-fluorouracil (5-FU) and leucovorin (LV) for patients with metastatic carcinoma of the colon or rectum. However, CAMPTOSAR® has not been approved for the treatment of other cancer types, such as pancreatic cancers. Topoisomerase I inhibitors such as irinotecan work to arrest uncontrolled cell growth by inhibiting the unwinding of DNA and thereby preventing DNA replication.

The pharmacology of irinotecan is complex, with extensive metabolic conversions involved in the activation, inactivation, and elimination of the drug. Irinotecan is a prodrug that is converted by nonspecific carboxylesterases into a 100-1000 fold more active metabolite, SN-38. SN-38 is not recognized by P-glycoprotein, a drug transporter that plays an important role in acquired drug resistance by pumping certain drugs out of cells, so irinotecan is likely to be active in tumors resistant to other standard chemotherapies. In the body, SN-38 is cleared via glucuronidation, for which major pharmacogenetic variability has been described, and biliary excretion. These drug properties contribute to the marked heterogeneities in efficacy and toxicity observed clinically with irinotecan. Irinotecan hydrochloride injection is approved in the United States for treatment of metastatic colon or renal cancer and is also used to treat colorectal, gastric, lung, uterine cervical and ovarian cancers.

There are few approved treatment options for advanced or metastatic pancreatic cancers, particularly for those of exocrine origin, and no FDA-approved treatments for pancreatic cancer following disease progression after first-line chemotherapy treatment. Gemcitabine has been approved as a single agent for the treatment of pancreatic cancer, and in combination with paclitaxel, for first-line treatment of metastatic breast cancer after failure of prior anthracycline-containing adjuvant chemotherapy, unless anthracyclines were clinically contraindicated. There remains an unmet clinical need for FDA-approved therapies to safely and effectively treat patients with pancreatic cancer, including metastatic adenocarcinoma of the pancreas, when the disease progresses following gemcitabine-based first-line therapy.

### SUMMARY

Provided are methods for treating pancreatic cancer in a patient (i.e., a human patient) comprising administering to the patient liposomal irinotecan (e.g., irinotecan sucrose octasulfate salt liposome injection, also referred to as MM-398) alone or in combination with 5-fluorouracil (5-FU) and leucovorin (together, 5-FU/LV), according to a particular clinical dosage regimen. Compositions adapted for use in such methods are also provided.

In one aspect, a method for treatment of pancreatic cancer in a patient is provided, the method comprising co-administering to the patient an effective amount each of liposomal irinotecan, 5-fluorouracil (5-FU), and leucovorin, wherein the method comprises at least one cycle of administration, wherein the cycle is a period of 2 weeks, and wherein for each cycle:

(a) liposomal irinotecan is administered to patients not homozygous for the UGT1A1*28 allele on day 1 of each cycle at a dose of 80 mg/m², and to patients homozygous for the UGT1A1*28 allele on day 1 of cycle 1 at a dose of 60 mg/m² and on day 1 of each subsequent cycle at a dose of ranging from 60 mg/m² to 80 mg/m² (e.g., 60 mg/m² or 70 mg/m² or 80 mg/m²);

(b) 5-FU is administered at a dose of 2400 mg/m²; and

(c) leucovorin is administered at a dose of 200 mg/m² (1 form, or levoleucovorin) or 400 mg/m² (l+d racemic form).

In another aspect, a formulation of liposomal irinotecan for co-administration with 5-fluorouracil (5-FU) and leucovorin in at least one cycle is provided, wherein the cycle is a period of 2 weeks, the formulation of irinotecan is a liposomal formulation of irinotecan, and wherein:

(a) liposomal irinotecan is administered to patients not homozygous for the UGT1A1*28 allele on day 1 of each cycle at a dose of 80 mg/m² and to patients homozygous for the UGT1A1*28 allele on day 1 of cycle 1 at a dose of 60 mg/m² and on day 1 of each subsequent cycle at a dose of 60 mg/m² or 80 mg/m²;
(b) 5-FU is administered at a dose of 2400 mg/m²; and
(c) leucovorin is administered at a dose of 200 mg/m² (1 form, or levoleucovorin) or 400 mg/m² (l+d racemic form).

In one embodiment, after cycle 1 the dose of liposomal irinotecan administered to the patient homozygous for the UGT1A1*28 allele is increased to 80 mg/m². In another embodiment, the liposomal irinotecan is administered intravenously over 90 minutes. In another embodiment, the 5-FU is administered intravenously over 46 hours. In another embodiment, leucovorin is administered intravenously over 30 minutes.

In another embodiment, prior to each administration of liposomal irinotecan, the patient is pre-medicated with dexamethasone and/or a 5-HT3 antagonist or another anti-emetic.

In another embodiment, the liposomal formulation of irinotecan is irinotecan sucrose octasulfate salt liposome injection. MM-398 is irinotecan liposome injection recently approved by FDA under the tradename ONIVYDE® (irinotecan liposome injection). MM-398 irinotecan liposome injection contains the topoisomerase 1 inhibitor irinotecan encapsulated with sucrose octasulfate in a lipid bilayer vesicle or liposome and formulated for intravenous administration. MM-398 is indicated in combination with fluorouracil and leucovorin for the treatment of patients with metastatic adenocarcinoma of the pancreas whose disease has progressed following gemcitabine-based therapy.

Provided herein, is a safe and effective therapy using an irinotecan liposome formulation for the treatment of metastatic adenocarcinoma of the pancreas in patients who have been previously treated with gemcitabine-based therapy. The therapy comprises the administration to the patient having pancreatic cancer once every two weeks: 70 mg/m² of irinotecan encapsulated in a MM-398 irinotecan liposome in combination with a therapeutically effective amount of the antineoplastic agent leucovorin and 2,400 mg/m² of the antineoplastic agent 5-fluorouracil, without administering any other antineoplastic agents for the treatment of the pancreatic cancer.

The therapy can be safely and effectively administered to patients diagnosed with metastatic adenocarcinoma of the pancreas after disease progression following gemcitabine-based therapy. The amount of leucovorin administered can be selected to provide a desired effect of the 5-fluorouracil (e.g., an amount of leucovorin comprising 200 mg/m² of levoleucovorin, such as 400 mg/m² of the (l+d) racemic form of leucovirin). For example, the patient can be treated with an antineoplastic therapy (referred to herein as “MM-398+5-FU/LV (MM-398 70 mg/m² q2w regimen)”) comprising: 70 mg/m² of irinotecan encapsulated in a MM-398 irinotecan liposome (e.g., as a 90 minute intravenous infusion) followed by 400 mg/m² of the antineoplastic agent (l+d) racemic leucovorin (e.g., as a 30 minute intravenous infusion) followed by 2,400 mg/m² of the antineoplastic agent 5-fluorouracil (e.g., as an intravenous infusion over 46 hours), without administering any other antineoplastic agents for the treatment of the pancreatic cancer (e.g., without administering gemcitabine).

Unless otherwise indicated, recitation of the amount of the irinotecan administered herein refers to the amount of irinotecan free base administered (molecular weight of about 587 g/mol). When indicated, the amount of irinotecan can also be expressed as the amount of irinotecan hydrochloride trihydrate (molecular weight of about 677 g/mol) providing a given amount of irinotecan free base: for example, 80 mg/m² of irinotecan hydrochloride trihydrate (e.g., as approved under the non-liposomal irinotecan product CAMPTOSAR®) contains about the same amount of irinotecan as a dose of 70 mg/m² of irinotecan in the MM-398 liposome formulation.

The therapy was evaluated in the NAPOLI-1 human clinical trial described herein, an international randomized Phase 3 trial in metastatic pancreatic cancer patients previously treated with a gemcitabine based therapy. In the NAPOLI-1 human clinical trial, analysis of the ITT (intent to treat) patient group demonstrated statistically significant increase in overall survival (OS) of MM-398+5-FU/LV (MM-398 70 mg/m² q2w regimen) over 5-FU/LV alone (FIGS. 4A and 4B) of 6.1 months versus 4.2 months respectively. Significant increase in OS was also observed in Progression Free Survival (PFS), Objective Response Rate (ORR), and Tumor Marker Response (CA 19-9) response (FIG. 5). In comparison, MM-398, as a single agent (120 mg/m² q3w regimen), did not show a significant difference in OS. In addition, a Forest plot sensitivity analyses of the NAPOLI-1 human clinical trial favored MM-398+5-FU/LV over 5-FU/LV across prognostic subgroups, tumor characteristics and previous treatment (FIGS. 6 and 7). In the Per Protocol (PP) population (described in FIG. 8) of patients in the NAPOLI-1 human clinical trial (patients receiving 6 weeks of treatment), the MM-398+5-FU/LV combination regimen achieved a median OS of 8.9 months versus 5.1 months in the 5-FU/LV arm (stratified Hazard Ratio (HR): 0.47, p=0.0018; FIGS. 9A and 9B). The observed patient safety profile was manageable, with most frequent being grade≧3 adverse events including neutropenia, fatigue and GI effects, such as diarrhea and vomiting (FIG. 12).

### BRIEF DESCRIPTION OF THE DRAWINGS

FIG. 1 is a graph providing the overall survival of patients in an assessment of the clinical efficacy and safety of the irinotecan liposome injection monotherapy or the irinotecan liposome injection in combination with 5-fluorouracil and leucovorin (the irinotecan liposome injection+5-FU/LV), compared to an active control arm of 5-FU/LV.

FIG. 2 is a flow-chart representation of the NAPOLI-1 study design.

FIG. 3 is a table providing the baseline characteristics of the Intent to Treat (ITT) (all randomized patients) population. In the table, CA 19-9 at baseline was unknown in 3% of the patients.

FIGS. 4A and 4B are graphical representations of overall survival (ITT population) in MM-398+5-FU/LV vs. MM-398 or 5-FU/LV alone. The data presented is the protocol-defined primary analysis data cut after 305 events. “**” denotes un-stratified HR: 0.67 (0.49-0.92), p=0.0122; “***” denotes un-stratified HR: 0.99 (0.77-1.28), p=0.9416.

FIG. 5 is a table providing tumor response and control data (protocol-defined primary analysis data cut). Overall Response Rate calculated per RECIST version 1.1. The CA 19-9 reduction response is defined as ≧50% reduction in baseline CA 19-9 levels, in patients with baseline levels>30 U/mL, and at least one post-baseline CA 19-9 measurement.

FIG. 6 is a forest plot of the overall survival in patients receiving MM-398+5FU/LV treatment vs. treatment with 5FU/LV alone. The results presented in the table are a data cut from a protocol-defined primary analysis by un-stratified log-rank test.

FIG. 7 is another forest plot of the overall survival in patients receiving MM-398+5FU/LV treatment vs. treatment with 5FU/LV alone. The results presented in the table are a data cut from a protocol-defined primary analysis by un-stratified log-rank test.

FIG. 8 is a flow chart explaining the ITT (all randomized patients) and PP (Per Protocol) populations. The Per Protocol population comprises eligible patients who received≧80% dose density of the protocol defined treatment during the first 6 weeks of treatment.

FIGS. 9A and 9B are graphical representations of overall survival (OS) for the Per Protocol (PP) patient population vs. the Non-Per Protocol (Non-PP) patient population. The results presented in the figures are a data cut from a protocol-defined primary analysis. Per protocol population was defined as patients who received at least 80% of the protocol defined treatment during the first 6 weeks of treatment and did not have protocol deviations related to inclusion/exclusion criteria, receiving prohibited therapies or not receiving treatment as randomized. FIG. 9A represents the PP population and FIG. 9B represents the Non-PP population.

FIG. 10 is a table providing the demographic characteristics for the Per Protocol (PP) patient population vs. the Non-Per Protocol (Non-PP) patient population. CA 19-9 includes only patients who had a measured CA 19-9 prior to treatment. “**” denotes results that showed a statistically significant difference (p value≦0.01). “***” denotes the median (1″ quartile, 3^{rd} quartile).

FIG. 11 is a table providing the dose modifications and treatment exposure. Duration of exposure is the time from (the date of the last administration of the study drug+the projected days to the next dose of the study drug administration—the date of the first study drug administration)/7

FIG. 12 is a table providing the safety data (adverse events) for the study. In the table, (1) the safety population refers to those patients receiving at least one dose of the study drug; (2) percentages of the populations having adverse events (AE's) were provided per CTCAE Version 4; and (3) hematologic adverse events include only those patients who had at least one post-baseline assessment.

FIG. 13 is a pictorial representation of nanoliposomal irinotecan (nal-IRI).

FIG. 14 provides a pharmacokinetic analysis of the extended circulation of irinotecan and the SN38 metabolite after administration of irinotecan within the liposome vs. irinotecan HCl, in patients with gastric cancer.

FIG. 15 provides irinotecan and SN38 levels in tumor tissue and plasma 72 hours after treatment with nal-IRI.

FIG. 16A is a graphical representation of the Overall Survival Rate (OS) corresponding to treatment with nal-IRI+5-FU/LV vs. 5-FU/LV alone.

FIG. 16B is a graphical representation of the Overall Survival Rate (OS) corresponding to treatment with nal-IRI vs. 5-FU/LV alone.

FIG. 17A is a graphical representation of the Progression Free Survival Rate (PFS) corresponding to treatment with nal-IRI+5-FU/LV vs. 5-FU/LV alone.

FIG. 17B is a graphical representation of the Progression Free Survival Rate (PFS) corresponding to treatment with nal-IRI vs. 5-FU/LV alone.

FIG. 18 is a Kaplan-Meier graph of Overall Survival (OS) in the NAPOLI-1 trial.

FIG. 19 is a bar graph that provides the association of baseline CA 19-9 levels with Overall Survival (OS) for patients treated with Nal-IRI+5-FU/LV (shaded bars) or 5-FU/LV (open bars), Hazard Ratios (HR) are shown with their 95% CIs.

FIG. 20 is a bar graph that provides the association of baseline CA 19-9 levels with Progression Free Survival Rate (PFS) for patients treated with Nal-IRI+5-FU/LV (shaded bars) or 5-FU/LV (open bars), Hazard Ratios (HR) are shown with their 95% CIs.

FIG. 21 is a graph showing the anti-tumor activity of MM-398 in an orthotopic pancreatic tumor model expressing luciferase (L3.6pl).

FIG. 22 is a graph showing accumulation of SN-38 in tumors following treatment with free irinotecan or liposomal irinotecan (MM-398).

FIG. 23 is a graph showing the effect of MM-398 on Carbonic Anhydrase IX Staining in a HT29 Xenograft Model.

FIG. 24 shows the effect of MM-398 on perfusion of small molecule Hoechst stain.

FIG. 25 summarizes the pharmacokinetics of MM-398 in q3w (irinotecan, liposome+free drug).

FIG. 26 summarizes the pharmacokinetics of MM-398 in q3w.

FIG. 27 is a trial profile.

FIG. 28 is a table providing demographics and baseline characteristics (PRO Population).

FIG. 29 is a graphical representation of the proportion of patients demonstrating improvement, stability, or worsening in global health status and functional scale scores (nal-IRI+5-FU/LV, n=71; 5-FU/LV, n=57).

FIG. 30 is a graphical representation of the proportion of patients demonstrating improvement, stability, or worsening in symptom scale scores (nal-IRI+5-FU/LV, n=71; 5-FU/LV, n=57).

FIG. 31 is a graphical representation of the median change from baseline to week 12 in global health status and functional scale scores (nal-IRI+5-FU/LV, n=71 at baseline; 5-FU/LV, n=57 at baseline).

FIG. 32 is a graphical representation of the median change from baseline to week 12 in symptom scale scores (nal-IRI+5-FU/LV, n=71 at baseline; 5-FU/LV, n=57 at baseline).

FIG. 33 is a table providing Treatment-Emergent Adverse Events From the Primary Analysis of the NAPOLI-1 Trial.

FIG. 34 is a table providing Demographics and Baseline Characteristics (Safety Population).

FIG. 35 is a table providing TEAEs by Age.

FIG. 36 is a table providing TEAEs by Ethnicity.

FIG. 37 is a table providing TEAEs by UGT1A1*28 Allele (TA7/TA7 Genotype).

FIG. 38 is a table providing TEAEs by Albumin Level.

FIG. 39 is a table providing TEAEs by KPS Score.

### DETAILED DESCRIPTION

As provided herein, irinotecan is administered in a stable liposomal formulation as irinotecan sucrose sulfate liposome injection (otherwise termed “irinotecan sucrose octasulfate salt liposome injection” or “irinotecan sucrosofate liposome injection”), the formulation referred to herein as “MM-398” (also known as PEP02, see U.S. Pat. No. 8,147,867). MM-398 may be provided as a sterile, injectable parenteral liquid for intravenous injection. The required amount of MM-398 may be diluted, e.g., in 500 mL of 5% dextrose injection USP and infused over a 90 minute period.

An MM-398 liposome is a unilamellar lipid bilayer vesicle of approximately 80-140 nm in diameter that encapsulates an aqueous space which contains irinotecan complexed in a gelated or precipitated state as a salt with sucrose octasulfate. The lipid membrane of the liposome is composed of phosphatidylcholine, cholesterol, and a polyethyleneglycol-derivatized phosphatidyl-ethanolamine in the amount of approximately one polyethyleneglycol (PEG) molecule for 200 phospholipid molecules.

This stable liposomal formulation of irinotecan has several attributes that may provide an improved therapeutic index. The controlled and sustained release improves activity of this schedule-dependent drug by increasing duration of exposure of tumor tissue to drug, an attribute that allows it to be present in a higher proportion of cells during the S-phase of the cell cycle, when DNA unwinding is required as a preliminary step in the DNA replication process. The long circulating pharmacokinetics and high intravascular drug retention in the liposomes can promote an enhanced permeability and retention (EPR) effect. EPR allows for deposition of the liposomes at sites, such as malignant tumors, where the normal integrity of the vasculature (capillaries in particular) is compromised resulting in leakage out of the capillary lumen of particulates such as liposomes. EPR may thus promote site-specific drug delivery of liposomes to solid tumors. EPR of MM-398 may result in a subsequent depot effect, where liposomes accumulate in tumor associated macrophages (TAMs), which metabolize irinotecan, converting it locally to the substantially more cytotoxic SN-38. This local bioactivation is believed to result in reduced drug exposure at potential sites of toxicity and increased exposure at cancer cells within the tumor.

In one embodiment, the liposomal irinotecan is liposomal irinotecan such as MM-398. MM-398 irinotecan liposome injection is a topoisomerase 1 inhibitor encapsulated in a lipid bilayer vesicle or liposome. Topoisomerase 1 relieves torsional strain in DNA by inducing single-strand breaks. Irinotecan and its active metabolite SN-38 bind reversibly to the topoisomerase 1-DNA complex and prevent re-ligation of the single-strand breaks, leading to exposure time-dependent double-strand DNA damage and cell death. In mice bearing human tumor xenografts, irinotecan liposome administered at irinotecan HCl-equivalent doses 5-fold lower than irinotecan HCl achieved similar intratumoral exposure of SN-38.

MM-398 is a topoisomerase inhibitor, formulated with irinotecan hydrochloride trihydrate into a liposomal dispersion, for intravenous use. The chemical name of irinotecan hydrochloride trihydrate is (5) 4,11-diethyl-3,4,12,14-tetrahydro-4-hydroxy-3,14-dioxo1H-pyrano[3′,4′:6,7]-indolizino[1,2 b]quinolin-9-yl-[1,4′bipiperidine]-1′-carboxylate, monohydrochloride, trihydrate. The empirical formula is C₃₃H₃₈N₄O₆.HCl.3H₂O and the molecular weight is 677.19 g/mole. The molecular structure is:

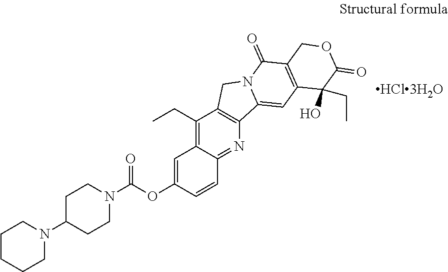 [SMILES: CCc1c2c(nc3ccc(OC(=O)N4CCC(N5CCCCC5)CC4)cc13)-c1cc3c(c(=O)n1C2)COC(=O)[C@]3(O)CC]

The chemical name of irinotecan is (5) 4,11-diethyl-3,4,12,14-tetrahydro-4-hydroxy-3,14-dioxo1H-pyrano[3′,4′: 6,7]-indolizino[1,2b]quinolin-9-yl-[1,4′bipiperidine]-1′-carboxylate. The empirical formula is C₃₃H₃₈N₄O₆ and the molecular weight is 586.68 g/mole. The molecular structure of irinotecan is:

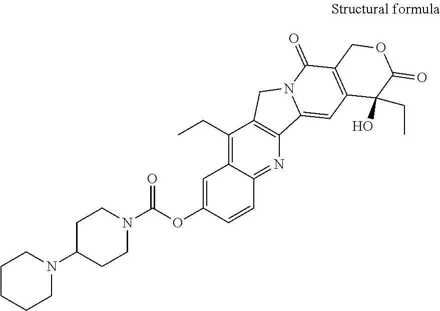 [SMILES: CCc1c2c(nc3ccc(OC(=O)N4CCC(N5CCCCC5)CC4)cc13)-c1cc3c(c(=O)n1C2)COC(=O)[C@]3(O)CC]

MM-398 is irinotecan liposome injection recently approved by FDA under the tradename ONIVYDE™ (irinotecan liposome injection). MM-398 is a topoisomerase I inhibitor indicated for the treatment of metastatic adenocarcinoma of the pancreas after disease progression following gemcitabine-based therapy. MM-398 (irinotecan liposome injection), in combination with fluorouracil and leucovorin, is indicated for the treatment of patients with metastatic adenocarcinoma of the pancreas whose disease has progressed following gemcitabine-based therapy. Administer MM-398 prior to leucovorin and fluorouracil. MM-398 is not indicated as a single agent for the treatment of metastatic adenocarcinoma of the pancreas. MM-398 is not substituted for other drugs containing non-liposome formulations of irinotecan hydrochloride or irinotecan hydrochloride trihydrate.

Converting a dose based on irinotecan hydrochloride trihydrate to a dose based on irinotecan free base is accomplished by multiplying the dose based on irinotecan hydrochloride trihydrate with the ratio of the molecular weight of irinotecan free base (586.68 g/mol) and the molecular weight of irinotecan hydrochloride trihydrate (677.19 g/mol). This ratio is 0.87 which can be used as a conversion factor. For example, an 80 mg/m² dose based on irinotecan hydrochloride trihydrate is equivalent to a 69.60 mg/m² dose based on irinotecan free base (80×0.87). In the clinic this is rounded to 70 mg/m².

As used herein, the dose of irinotecan liposome (including doses of MM-398 or PEP02 irinotecan liposome) expressed in mg/m² refers to the amount of irinotecan in the corresponding weight of irinotecan hydrochloride trihydrate unless otherwise indicated. For example, a dose of 80 mg/m² irinotecan liposome refers to a dose of the irinotecan liposome that provides 80 mg/m² irinotecan hydrochloride trihydrate (“hydrochloride salt dose”). Alternatively, the dose of irinotecan liposome can be expressed as the amount of irinotecan free base encapsulated in the irinotecan liposome (“free base dose”). In the ONIVYDE irinotecan liposome product, the dose of irinotecan liposome is expressed in terms of the free base dose of irinotecan. Unless otherwise indicated, the irinotecan liposome dose free base dose recited in the US Prescribing Information for ONIVYDE can be converted to the irinotecan liposome hydrochloride salt dose according to the table below.

TABLE 1

| Irinotecan liposome free base | Corresponding irinotecan liposome |
|---|---|
| dose in ONIVYDE USPI (mg/m²) | hydrochloride salt dose (mg/m²) |
| (“free base”) | (“salt”) |
| 100 | 120 |
| 70 | 80 |
| 60 | 70 |
| 50 | 60 |
| 43 | 50 |
| 35 | 40 |

The recommended dose of MM-398 is 80 mg/m² (salt) (70 mg/m² free base) intravenous infusion over 90 minutes (i.e., 70 mg irinotecan free base in the irinotecan liposome per m² patient body surface area, containing about the same amount of irinotecan that would be in 80 mg/m² of irinotecan hydrochloride trihydrate). Preferably, the recommended dose of MM-398 is 80 mg/m² administered by intravenous infusion over 90 minutes every 2 weeks. A corticosteroid and an anti-emetic is preferably administered to the patient 30 minutes prior to MM-398.

MM-398 can be obtained as the product ONIVYDE (irinotecan liposome for injection) (Merrimack Pharmaceuticals, Inc, Cambridge, Mass.), a sterile, white to slightly yellow opaque isotonic liposomal dispersion. Each 10 mL single-dose vial contains 43 mg irinotecan free base at a concentration of 4.3 mg/mL. The liposome is a unilamellar lipid bilayer vesicle, approximately 110 nm in diameter, which encapsulates an aqueous space containing irinotecan in a gelated or precipitated state as the sucrose-octasulfate salt, which is also referred to as the “sucrosofate salt.” The vesicle is composed of 1,2-distearoyl-sn-glycero-3-phosphocholine (DSPC) 6.81 mg/mL, cholesterol 2.22 mg/mL, and methoxy-terminated polyethylene glycol (MW 2000)-distearoylphosphatidyl ethanolamine (MPEG-2000-DSPE) 0.12 mg/mL. Each mL also contains 2-[4-(2-hydroxyethyl) piperazin-1-yl]ethanesulfonic acid (HEPES) as a buffer 4.05 mg/mL and sodium chloride as an isotonicity reagent 8.42 mg/mL.

To prepare MM-398, follow applicable special handling and disposal procedures. MM-398 can be provided as a single dose vial containing 43 mg of irinotecan in 10 mL single dose vial. The irinotecan is encapsulated in liposomes. Withdraw the calculated volume of MM-398 from the vial. Dilute MM-398 in 500 mL 5% Dextrose Injection, USP or 0.9% Sodium Chloride Injection, USP and mix diluted solution by gentle inversion. Protect diluted solution from light. Administer diluted solution within 4 hours of preparation when stored at room temperature or within 24 hours of preparation when stored under refrigerated conditions [2° C. to 8° C. (36° F. to 46° F.)]. Allow diluted solution to come to room temperature prior to administration. Do not freeze. Infuse diluted solution intravenously over 90 minutes. Do not use in-line filters. Discard unused portion. Injection: 43 mg/10 mL irinotecan free base as a white to slightly yellow, opaque, liposomal dispersion in a single-dose vial. MM-398 is available in a single-dose vial containing 43 mg irinotecan free base at a concentration of 4.3 mg/mL NDC: 69171-398-01. Refrigerate MM-398 at 2° C. to 8° C. (36° F. to 46° F.). Do NOT freeze. Protect from light. MM-398 is a cytotoxic drug. Follow applicable special handling and disposal procedures.

### MM-398 Dosage and Administration

Liposomal irinotecan can be administered in combination with 5-fluorouracil and leucovorin to treat pancreatic cancer. The pancreatic cancer can be an exocrine pancreatic cancer selected from the group consisting of acinar cell carcinoma, adenocarcinoma, adenosquamous carcinoma, giant cell tumor, intraductal papillary-mucinous neoplasm (IPMN), mucinous cystadenocarcinoma, pancreatoblastoma, serous cystadenocarcinoma, and solid and pseudopapillary tumors. In one embodiment, a patient treated using the methods and compositions disclosed herein exhibits evidence of recurrent or persistent pancreatic cancer following primary chemotherapy. In another embodiment, the patient has had and failed at least one prior platinum based chemotherapy regimen for management of primary or recurrent disease, e.g., a chemotherapy regimen comprising carboplatin, cisplatin, or another organoplatinum compound. In an additional embodiment, the patient has failed prior treatment with gemcitabine or become resistant to gemcitabine. In one embodiment a resistant or refractory tumor is one where the treatment-free interval following completion of a course of therapy for a patient having the tumor is less than 6 months (e.g., owing to recurrence of the cancer) or where there is tumor progression during the course of therapy. In another embodiment, the pancreatic cancer of the patient undergoing treatment is advanced pancreatic cancer, which is a pancreatic tumor that exhibits either or both of distant metastasis or peripancreatic extension of the tumor. The compositions and methods disclosed herein are useful for the treatment of all pancreatic cancers, including pancreatic cancers that are refractory or resistant to other anti-cancer treatments.

In one embodiment, liposomal irinotecan is administered prior to 5-FU and leucovorin to treat pancreatic cancer. In another embodiment, leucovorin is administered prior to 5-FU. In another embodiment, liposomal irinotecan is administered intravenously over 90 minutes. In another embodiment, 5-FU is administered intravenously over 46 hours. In another embodiment, leucovorin is administered intravenously over 30 minutes. In various embodiments the liposomal irinotecan is MM-398.

In another aspect, a formulation of liposomal irinotecan for co-administration with 5-fluorouracil (5-FU) and leucovorin in at least one cycle is provided, wherein the cycle is a period of 2 weeks, the formulation of irinotecan is a liposomal formulation of irinotecan, and wherein:

(a) liposomal irinotecan is administered to patients not homozygous for the UGT1A1*28 allele on day 1 of each cycle at a dose of 80 mg/m² and to patients homozygous for the UGT1A1*28 allele on day 1 of cycle 1 at a dose of 60 mg/m² and on day 1 of each subsequent cycle at a dose of 60 mg/m² or 80 mg/m²;

(b) 5-FU is administered at a dose of 2400 mg/m²; and

(c) leucovorin is administered at a dose of 200 mg/m² (1 form, or levoleucovorin) or 400 mg/m² (l+d racemic form).

In another embodiment, the 5-FU is administered intravenously over 46 hours.

In another embodiment, leucovorin is administered intravenously over 30 minutes.

In another embodiment, prior to each administration of liposomal irinotecan, the patient is pre-medicated with dexamethasone and/or a 5-HT3 antagonist or another anti-emetic.

5-Fluorouracil is a pyrimidine antagonist that interferes with nucleic acid biosynthesis. The deoxyribonucleotide of the drug inhibits thymidylate synthetase, thus inhibiting the formation of thymidylic acid from deoxyuridylic acid, thus interfering in the synthesis of DNA. It also interferes with RNA synthesis.

Leucovorin (also called folinic acid) acts as a biochemical cofactor for 1-carbon transfer reactions in the synthesis of purines and pyrimidines. Leucovorin does not require the enzyme dihydrofolate reductase (DHFR) for conversion to tetrahydrofolic acid. The effects of methotrexate and other DHFR-antagonists are inhibited by leucovorin. Leucovorin can potentiate the cytotoxic effects of fluorinated pyrimidines (i.e., fluorouracil and floxuridine). After 5-FU is activated within the cell, it is accompanied by a folate cofactor, and inhibits the enzyme thymidylate synthetase, thus inhibiting pyrimidine synthesis. Leucovorin increases the folate pool, thereby increasing the binding of folate cofactor and active 5-FU with thymidylate synthetase.

Leucovorin has dextro- and levo-isomers, only the latter one being pharmacologically useful. As such, the bioactive levo-isomer (“levoleucovorin”) has also been approved by the FDA for treatment of cancer. The dosage of levoleucovorin is typically half that of the racemic mixture containing both dextro (d) and levo (l) isomers.

FU and leucovorin will be stored and handled according to the country specific package inserts.

The enzyme produced by the UGT1A1 gene, UDP-glucuronosyltransferase 1, is responsible for bilirubin metabolism and also mediates SN-38 glucuronidation, which is the initial step in the predominant metabolic clearance pathway of this active metabolite of irinotecan. Besides its anti-tumor activity, SN-38 is also responsible for the severe toxicity sometimes associated with irinotecan therapy. Therefore, the glucuronidation of SN-38 to the inactive form, SN-38 glucuronide, is an important step in the modulation of irinotecan toxicity.

Mutational polymorphisms in the promoter of the UGT1A1 gene have been described in which there is a variable number of thymine adenine (ta) repeats. Promoters containing seven thymine adenine (ta) repeats (found in the UGT1A1*28 allele) have been found to be less active than the wild-type six repeats, resulting in reduced expression of UDP-glucuronosyltransferase 1. Patients who carry two deficient alleles of UGT1A1 exhibit reduced glucuronidation of SN-38. Some case reports have suggested that individuals who are homozygous for UGT1A1*28 alleles (referred to as having the UGT1A1 7/7 genotype, because both alleles are UGT1A1*28 alleles that contain 7 ta repeats, as opposed to the wild-type UGT1A1 6/6 genotype in which both alleles contain 6 ta repeats) and who have fluctuating elevation in serum bilirubin, (e.g., Gilbert's Syndrome patients), may be at greater risk of toxicity upon receiving standard doses of irinotecan. This suggests that there is a link between homozygosity of the UGT1A1*28 allele, bilirubin levels and irinotecan toxicity.

The metabolic transformation of MM-398 to SN-38 (e.g., in plasma) includes two critical steps: (1) the release of irinotecan from the liposome and (2) the conversion of free irinotecan to SN-38. While not intending to be limited by theory, it is believed that once irinotecan leaves the liposomes, it is catabolized by the same metabolic pathways as conventional (free) irinotecan. Therefore the genetic polymorphisms in humans predictive for the toxicity and efficacy of irinotecan and those of MM-398 can be considered similar. Nonetheless, due to the smaller tissue distribution, lower clearance, higher systemic exposure and longer elimination half-life of SN-38 of the MM-398 formulation compared to free irinotecan, the deficient genetic polymorphisms may show more association with severe adverse events and/or efficacy.

Individuals who are homozygous for the UGT1A1*28 allele (UGT1A1 7/7 genotype) have been shown to be at increased risk for neutropenia following initiation of irinotecan treatment. According to the prescribing information for irinotecan (Camptosar®), in a study of 66 patients who received single-agent irinotecan (350 mg/m2 once every-3-weeks), the incidence of grade 4 neutropenia in patients homozygous for the UGT1A1*28 allele was as high as 50%, and in patients heterozygous for this allele (UGT1A1 6/7 genotype) the incidence was 12.5%. Importantly, no grade 4 neutropenia was observed in patients homozygous for the wild-type allele (UGT1A1 6/6 genotype). In other studies, a lower prevalence of life threatening neutropenia is described. For this reason, patients who are enrolled in the phase 3 study described in the Examples herein and are homozygous for the UGT1A1*28 allele (UGT1A1 7/7 genotype) will have MM-398 treatment initiated at a lower dose than patients with one (e.g., UGT1A1 6/7) or two (UGT1A1 6/6) wild-type alleles.

Although the UGT1A1*28 allele is relatively common in Caucasians (estimates 10%), the prevalence is varied in other ethnic groups. Furthermore, additional UGT1A1 genotypes are found with higher prevalence for example in Asian populations and these could be important for the metabolism of irinotecan in these populations. For example, the UGT1A1*6 allele is more prevalent in Asians. This allele is not associated with a to repeat, but with a Gly71Arg mutation that reduces enzyme activity. In previous and ongoing studies of MM-398, pharmacogenetic information has been collected on patients being enrolled. In a study referred to as the PEP0203 study, the relationship of genetic polymorphism of UGT1A family and of DPYD (dihydropyrimidine dehydrogenase, an enzyme associated with catabolism of 5-FU) with pharmacokinetic parameters of MM-398 and toxicity did not provide a clear correlation with the small sample size of subjects evaluated. However, it was observed that patients with UGT1A1*6/*28 combined polymorphism had higher dose-normalized AUCs of SN-38 and experienced DLT.

The recommended starting dose of MM-398 in patients known to be homozygous for the UGT1A1*28 allele is 50 mg/m² administered by intravenous infusion over 90 minutes. Increase the dose of MM-398 to 70 mg/m² as tolerated in subsequent cycles. In one embodiment, after cycle 1 the dose of liposomal irinotecan administered to the patient homozygous for the UGT1A1*28 allele is increased to 80 mg/m². In another embodiment, the liposomal irinotecan is administered intravenously over 90 minutes.

In some embodiments, methods of administering MM-398 to patients having one or more characteristics can include reducing or otherwise modifying the dose of MM-398 administered according to the embodiments herein. In some embodiments, the dose of MM-398 is modified according to Table 2.

TABLE 2
Recommended Dose Modifications for MM-398

| | | MM-398 | Patients homozygous |
|---|---|---|---|
| | | adjustment | for UGT1A1*28 |
| Toxicity | | in patients | without previous |
| NCI CTCAE | | receiving | increase to |
| v4.0 | Occurrence | 70 mg/m^{2‡} | 70 mg/m² |
| Grade 3 or 4 | Withhold MM-398. | | |
| adverse | Initiate loperamide for late onset diarrhea of any severity. | | |
| reactions | Administer intravenous or subcutaneous atropine 0.25 to | | |
| | 1 mg (unless clinically contraindicated) for early onset | | |
| | diarrhea of any severity. | | |
| | Upon recovery to ≦Grade 1 or baseline grade resume | | |
| | MM-398 at: | | |
| | First | 50 mg/m² | 43 mg/m² |
| | Second | 43 mg/m² | 35 mg/m² |
| | Third | Discontinue | Discontinue |
| | | MM-398 | MM-398 |
| Interstitial | First | Discontinue | Discontinue |
| Lung | | MM-398 | MM-398 |
| Disease | | | |
| Anaphylactic | First | Discontinue | Discontinue |
| Reaction | | MM-398 | MM-398 |

Irinotecan is subject to extensive metabolic conversion by various enzyme systems, including esterases to form the active metabolite SN-38, and UGT1A1 mediating glucuronidation of SN-38 to form the inactive glucuronide metabolite SN-38G. Irinotecan can also undergo CYP3A4-mediated oxidative metabolism to several inactive oxidation products, one of which can be hydrolyzed by carboxylesterase to release SN-38. In the population pharmacokinetic analysis using the results of a subset with UGT1A1*28 genotypic testing, in which the analysis adjusted for the lower dose administered to patients homozygous for the UGT1A1*28 allele, patients homozygous (N=14) and non-homozygous (N=244) for this allele had total SN-38 average steady-state concentrations of 1.06 [95% CI: 0.9-1.25] and 0.95 [95% CI: 0.9-0.99] ng/mL, respectively.

Individuals who are homozygous for the UGT1A1*28 allele are at increased risk for neutropenia from irinotecan HCl. In Study 1, patients homozygous for the UGT1A1*28 allele (n=7) initiated MM-398 at a reduced dose of 50 mg/m² in combination with 5-FU/LV. The frequency of Grade 3 or 4 neutropenia in these patients [2 of 7 (28.6%)] was similar to the frequency in patients not homozygous for the UGT1A1*28 allele who received a starting dose of MM-398 of 70 mg/m² [30 of 110 (27.3%)].

### Pharmacokinetics of MM-398

Preferably, the irinotecan liposome is characterized by the pharmacokinetic parameters in table 3 (e.g., MM-398 irinotecan liposome), or is bioequivalent to such an irinotecan liposome (e.g., an irinotecan liposome characterized by one or more values that are within 80%-105% of one or more of the values for parameters Cₘₐₓ, AUC and t_{1/2} in table 3 below), when administered once every two weeks to a human patient.

The plasma pharmacokinetics of total irinotecan and total SN-38 were evaluated in patients with cancer who received MM-398, as a single agent or as part of combination chemotherapy, at doses between 50 and 150 mg/m² and 353 patients with cancer using population pharmacokinetic analysis. The pharmacokinetic parameters of total irinotecan and total SN-38 following the administration of MM-398 70 mg/m² as a single agent or part of combination chemotherapy are presented in Table 3, or have a Cmax and AUC value within 80-125% of the value observed from MM-398 administered at a dose of 80 mg/m² (salt) q2w. Summary of Mean (±Standard Deviation)

TABLE 3
Total Irinotecan and Total SN-38
Pharmacokinetic Parameters in Patients with Solid Tumors.

| | Total Irinotecan | | | | | Total SN-38 | | |
|---|---|---|---|---|---|---|---|---|
| | Cₘₐₓ | AUC_{0-∞} | t_{1/2} | CL | V_{d} | Cₘₐₓ | AUC_{0-∞} | t_{1/2} |
| Dose | [μg/mL] | [h · μg/mL] | [h] | [L/h] | [L] | [ng/mL] | [h · ng/mL] | [h] |
| (mg/m²) | (n = 25) | (n = 23) | (n = 23) | (n = 23) | (n = 23) | (n = 25) | (n = 13) | (n = 13) |
| Max | 46.5 | 1718 | | | | | | |
| (125%) | | | | | | | | |
| 80 | 37.2 | 1364 | 25.8 | 0.20 | 4.1 | 5.4 | 620 | 67.8 |
| | (8.8) | (1048) | (15.7) | (0.17) | (1.5) | (3.4) | (329) | (44.5) |
| Min | 29.8 | 1091 | | | | | | |
| (80%) | | | | | | | | |

Cₘₐₓ: Maximum plasma concentration
AUC_{0-∞}: Area under the plasma concentration curve extrapolated to time infinity
t_{1/2}: Terminal elimination half-life
V_{d}: Volume of distribution

The pharmacokinetic parameters of total Irinotecan and total SN-38 following administration of MM-398 70 mg/m² as a single agent or part of combination chemotherapy are presented in Table 3.

Over the dose range of 50 to 150 mg/m², the Cmax and AUC of total irinotecan increases with dose. Additionally, the Cₘₐₓ of total SN-38 increases proportionally with dose; however, the AUC of total SN-38 increases less than proportionally with dose. The correlation of SN-38 Cₘₐₓ with liposomal irinotecan dose had not previously been established. Higher plasma SN-38 Cₘₐₓ was associated with increased likelihood of experiencing neutropenia.

The Cₘₐₓ of SN-38 increases proportionally with liposomal irinotecan dose but the AUC of SN-38 increases less than proportionally with dose, enabling new methods of dosage adjustment. For example, the value of the parameter associated with adverse effects (Cₘₐₓ) decreases by a relatively greater extent than the value of the parameter associated with the effectiveness of treatment (AUC). Accordingly, when an adverse effect is seen, a reduction in the dosing of the liposomal irinotecan can be implemented that maximizes the difference between the reduction in Cₘₐₓ and in AUC. The discovery means that in treatment regimens, a given SN-38 AUC can be achieved with a surprisingly low SN-38 Cₘₐₓ. Likewise, a given SN-38 Cₘₐₓ can be achieved with a surprisingly high SN-38 AUC.

Direct measurement of irinotecan liposome showed that 95% of irinotecan remains liposome encapsulated, and the ratios between total and encapsulated forms did not change with time from 0 to 169.5 hours post-dose. The mean volume of distribution is summarized in Table 3.

In some embodiments, the liposomal irinotecan can be MM-398 or a product that is bioequivalent to MM-398. In some embodiments, the liposomal irinotecan can be characterized by the parameters in Table 4, including a Cmax and/or AUC value that is 80-125% of the corresponding value in Table 3. The pharmacokinetic parameters of total irinotecan for various alternative liposomal irinotecan formulations administering 70 mg/m² irinotecan free base once every two weeks is provided in Table 4.

TABLE 4
Total Irinotecan Pharmacokinetic Parameters in
Alternative Liposomal Irinotecan Formulations

Total Irinotecan

| | Cₘₐₓ | AUC_{0-∞} |
|---|---|---|
| Dose | [μg/mL] | [h · μg/mL] |
| (mg/m²) | (n = 25) | (n = 23) |
| 70 | 29.8-46.5 | 1091-1705 |

Cₘₐₓ: Maximum plasma concentration
AUC_{0-∞}: Area under the plasma concentration curve extrapolated to time infinity
t_{1/2}: Terminal elimination half-life

Plasma protein binding is <0.44% of the total irinotecan in MM-398.

The plasma clearance of total irinotecan from MM-398 70 mg/m² is 0.077 L/h/m² with a terminal half live of 26.8 h. Following administration of irinotecan HCl 125 mg/m², the plasma clearance of irinotecan is 13.3 L/h/m² with a terminal half live of 10.4 h.

The disposition of MM-398 has not been elucidated in humans. Following administration of irinotecan HCl, the urinary excretion of irinotecan is 11% to 20%; SN-38, <1%; and SN-38 glucuronide, 3%. The cumulative biliary and urinary excretion of irinotecan and its metabolites (SN-38 and SN-38 glucuronide), over a period of 48 hours following administration of irinotecan HCl in two patients, ranged from approximately 25% (100 mg/m²) to 50% (300 mg/m²).

The population pharmacokinetic analysis suggests that age (28 to 87 years) had no clinically meaningful effect on the exposure of irinotecan and SN-38.

The population pharmacokinetic analysis suggests that gender (196 males and 157 females) had no clinically meaningful effect on the exposure of irinotecan and SN-38 after adjusting for body surface area (BSA).

In a population pharmacokinetic analysis, mild-to-moderate renal impairment had no effect on the exposure of total SN-38 after adjusting for BSA. The analysis included 68 patients with moderate (CLcr 30-59 mL/min), 147 patients with mild (CLcr 60-89 mL/min) renal impairment, and 145 patients with normal renal function (CLcr>90 mL/min). There was insufficient data in patients with severe renal impairment (CLcr<30 mL/min) to assess its effect on pharmacokinetics.

The population pharmacokinetic analysis suggest that Asians (East Asians) have 56% lower total irinotecan average steady state concentration and 8% higher total SN-38 average steady state concentration than Whites.

The pharmacokinetics of irinotecan liposome have not been studied in patients with hepatic impairment. In a population pharmacokinetic analysis, patients with baseline bilirubin concentrations of 1-2 mg/dL (n=19) had average steady state concentrations for total SN-38 that were increased by 37% compared to patients with baseline bilirubin concentrations of <1 mg/dL (n=329); however, there was no effect of elevated ALT/AST concentrations on total SN-38 concentrations. No data are available in patients with bilirubin>2 mg/dL.

In a population pharmacokinetic analysis, the pharmacokinetics of total irinotecan and total SN-38 were not altered by the co-administration of fluorouracil/leucovorin.

Following administration of irinotecan HCl, dexamethasone, a moderate CYP3A4 inducer, does not alter the pharmacokinetics of irinotecan.

In vitro studies indicate that irinotecan, SN-38 and another metabolite, aminopentane carboxylic acid (APC), do not inhibit cytochrome P-450 isozymes.

### Safety and Tolerability

MM-398 is a cytotoxic drug. In some embodiments, methods of administering MM-398 comprise certain precautions. For example, patients can have or have had neutropenia, diarrhea, or interstitial lung disease.

In some embodiments, MM-398 is administered without causing severe or fatal neutropenia or neutropenic sepsis. MM-398 can cause severe and fatal neutropenia/neutropenic sepsis. In Study 1, the incidence of neutropenic sepsis was 0.8% among patients receiving MM-398, occurring in one of 117 patients in the MM-398 plus fluorouracil/leucovorin (MM-398/5-FU/LV) arm and one of 147 patients receiving MM-398 as a single agent. Severe or life threatening neutropenia occurred in 27% of patients receiving MM-398/5-FU/LV compared to 2% of patients receiving fluorouracil/leucovorin alone (5-FU/LV). Grade 3 or 4 neutropenic fever/neutropenic sepsis occurred in 3% of patients receiving MM-398/5-FU/LV, and did not occur in patients receiving 5-FU/LV.

In patients receiving MM-398/5-FU/LV, the incidence of Grade 3 or 4 neutropenia was higher among Asian patients [18 of 33 (55%)] compared to White patients [13 of 73 (18%)]. Neutropenic fever/neutropenic sepsis was reported in 6% of Asian patients compared to 1% of White patients.

Accordingly, in some embodiments, MM-398 is administered while monitoring complete blood cell counts on Days 1 and 8 of every cycle and more frequently if clinically indicated. Clinical indication includes an assessment of the likelihood of the patient developing neutropenia. Patients with elevated neutropenia risk include those with non-wildtype UGT1A1 and/or who are Asian. In some embodiments, the methods disclosed herein comprise the step of performing blood cell counts more frequently when the patient is Asian and/or has a non-wildtype UGT1A1 allele. In some embodiments, the blood cell counts are performed on day 1, on one or more of days 2-7, on day 8, and one or more of days 9-14, for example wherein the blood cell counts are performed on day 1, on two or more of days 2-7, on day 8, and two or more of days 9-14; wherein the blood cell counts are performed on day 1, on three or more of days 2-7, on day 8, and three or more of days 9-14; wherein the blood cell counts are performed on day 1, on four or more of days 2-7, on day 8, and four or more of days 9-14; wherein the blood cell counts are performed on day 1, on five or more of days 2-7, on day 8, and five or more of days 9-14; such as wherein the blood cell counts are performed on each of days 1-14. In some embodiments, the blood cell count detects neutropenia, for example wherein neutropenia is detected when absolute neutrophil count is below 1500/mm³. In some embodiments, the subsequent dose of liposomal irinotecan is reduced when neutropenia is detected. In some embodiments the reduced dose is not zero.

Withhold MM-398 if the absolute neutrophil count (ANC) is below 1500/mm³ or if neutropenic fever occurs. Resume MM-398 when ANC is 1500/mm³ or above. Reduce MM-398 dose for Grade 3-4 neutropenia or neutropenic fever following recovery in subsequent cycles. The determination of which reduced dose to use can incorporate a consideration of the discussion in paragraph [0018]. When neutropenia is observed, for example in an Asian patient, the dose of liposomal irinotecan can be reduced in order to reduce SN-38 Cₘₐₓ, which correlates with the adverse effect. Therapeutic effectiveness would be expected nonetheless, however, on the basis that the reduction in effect (based on AUC) would be surprisingly high in comparison. Thus, this discovery by the inventors means that treatment may be continued in many instances where previously clinicians may have determined it necessary to cease treatment entirely, on the prior assumption that SN-38 Cₘₐₓ and SN-38 AUC both related to liposomal irinotecan dose in the same fashion. In other words, the relationship between these parameters and liposomal irinotecan dosing is such that the reduction in liposomal irinotecan dose reduces SN-38 Cₘₐₓ below the threshold at which adverse effects occur, but maintains the SN-38 AUC above the threshold of at which therapeutic results are obtained. Accordingly, in some embodiments of the methods disclosed herein, the method comprises the step of reducing the dose of liposomal irinotecan when the patient is determined to have neutropenia (or to be suffering from another adverse effect) or be likely to developing neutropenia (or another adverse effect). In some embodiments the dose is reduced, but treatment with liposomal irinotecan is not stopped (i.e. the reduced dose is not zero).

By analysis of blood count and SN-38 Cₘₐₓ in a study population, it is possible to determine the relationship between neutropenia and SN-38 Cₘₐₓ, and thereby calculate a threshold level of Cₘₐₓ, which when exceeded, indicates neutropenia has developed or will likely develop. This threshold can be used as a predetermined threshold in subsequent tests on other patients. Accordingly, in some embodiments, the methods disclosed herein include a step of monitoring SN-38 Cₘₐₓ and where appropriate altering the dosage of liposomal irinotecan. As noted above, the relationship between SN-38 Cₘₐₓ and administered liposomal irinotecan is proportional, meaning that the required reduction in liposomal irinotecan dose can be calculated empirically from the extent that a patient's SN-38 Cₘₐₓ exceeds the threshold level known to be associated with neutropenia, but at which the AUC parameter exceeds the threshold at which a therapeutic effect is seen. Similar analyses can be implemented for countering the risk of interstitial lung disease or a severe hypersensitivity reaction before physical symptoms appear, and indeed other adverse effects.

In some embodiments, MM-398 is administered without causing severe or life-threatening diarrhea. MM-398 can cause severe and life-threatening diarrhea. Do not administer MM-398 to patients with bowel obstruction. Severe or life-threatening diarrhea followed one of two patterns: late onset diarrhea (onset more than 24 hours following chemotherapy) and early onset diarrhea (onset within 24 hours of chemotherapy, sometimes occurring with other symptoms of cholinergic reaction). An individual patient may experience both early and late-onset diarrhea.

In Study 1, Grade 3 or 4 diarrhea occurred in 13% receiving MM-398/5-FU/LV compared to 4% receiving 5-FU/LV. The incidence of Grade 3 or 4 late onset diarrhea was 9% in patients receiving MM-398/5-FU/LV, compared to 4% in patients receiving 5-FU/LV. The incidence of Grade 3 or 4 early onset diarrhea was 3% in patients receiving MM-398/5-FU/LV, compared to no Grade 3 or 4 early onset diarrhea in patients receiving 5-FU/LV. Of patients receiving MM-398/5-FU/LV in Study 1, 34% received loperamide for late-onset diarrhea and 26% received atropine for early-onset diarrhea.

In some embodiments, method of treatment comprise withholding MM-398 for Grade 2-4 diarrhea. Initiate loperamide for late onset diarrhea of any severity. Administer intravenous or subcutaneous atropine 0.25 to 1 mg (unless clinically contraindicated) for early onset diarrhea of any severity. Following recovery to Grade 1 diarrhea, resume MM-398 at a reduced dose.

In some embodiments, MM-398 is administered to a patient without causing severe or fatal Interstitial Lung Disease. Irinotecan HCl can cause severe and fatal interstitial lung disease (ILD). Withhold MM-398 in patients with new or progressive dyspnea, cough, and fever, pending diagnostic evaluation. Discontinue MM-398 in patients with a confirmed diagnosis of ILD.

In some embodiments, MM-398 is not administered to patients who experience severe hypersensitivity reaction to irinotecan. Irinotecan HCl can cause severe hypersensitivity reactions, including anaphylactic reactions. Permanently discontinue MM-398 in patients who experience a severe hypersensitivity reaction.

In some embodiments, MM-398 is not administered to pregnant or lactating patients. Based on animal data with irinotecan HCl and the mechanism of action of MM-398, MM-398 can cause fetal harm when administered to a pregnant woman. Embryotoxicity and teratogenicity were observed following treatment with irinotecan HCl, at doses resulting in irinotecan exposures lower than those achieved with MM-398 70 mg/m² in humans, administered to pregnant rats and rabbits during organogenesis. Advise pregnant women of the potential risk to a fetus. Advise females of reproductive potential to use effective contraception during treatment with MM-398 and for one month following the final dose. Based on animal data with irinotecan HCl and the mechanism of action of MM-398, MM-398 can cause fetal harm when administered to a pregnant woman. There are no available data in pregnant women. Embryotoxicity and teratogenicity were observed following treatment with irinotecan HCl, at doses resulting in irinotecan exposures lower than those achieved with MM-398 70 mg/m² in humans, administered to pregnant rats and rabbits during organogenesis. Advise pregnant women of the potential risk to a fetus.

In the U.S. general population, the estimated background risk of major birth defects and miscarriage in clinically recognized pregnancies is 2-4% and 15-20%, respectively. No animal studies have been conducted to evaluate the effect of irinotecan liposome on reproduction and fetal development; however, studies have been conducted with irinotecan HCl. Irinotecan crosses the placenta of rats following intravenous administration. Intravenous administration of irinotecan at a dose of 6 mg/kg/day to rats and rabbits during the period of organogenesis resulted in increased post-implantation loss and decreased numbers of live fetuses. In separate studies in rats, this dose resulted in an irinotecan exposure of approximately 0.002 times the exposure of irinotecan based on area under the curve (AUC) in patients administered MM-398 at the 70 mg/m² dose. Administration of irinotecan HCl resulted in structural abnormalities and growth delays in rats at doses greater than 1.2 mg/kg/day (approximately 0.0002 times the clinical exposure to irinotecan in MM-398 based on AUC). Teratogenic effects included a variety of external, visceral, and skeletal abnormalities. Irinotecan HCl administered to rat dams for the period following organogenesis through weaning at doses of 6 mg/kg/day caused decreased learning ability and decreased female body weights in the offspring.

There is no information regarding the presence of irinotecan liposome, irinotecan, or SN-38 (an active metabolite of irinotecan) in human milk, or the effects on the breastfed infant or on milk production. Irinotecan is present in rat milk. Because of the potential for serious adverse reactions in breastfed infants from MM-398, advise a nursing woman not to breastfeed during treatment with MM-398 and for one month after the final dose. Radioactivity appeared in rat milk within 5 minutes of intravenous administration of radiolabeled irinotecan HCl and was concentrated up to 65-fold at 4 hours after administration relative to plasma concentrations.

MM-398 can cause fetal harm when administered to a pregnant woman. Advise females of reproductive potential to use effective contraception during treatment with MM-398 and for one month after the final dose. Because of the potential for genotoxicity, advise males with female partners of reproductive potential to use condoms during treatment with MM-398 and for four months after the final dose.

There is no recommended dose of MM-398 for patients with serum bilirubin above the upper limit of normal. Safety and effectiveness of MM-398 have not been established in pediatric patients. There are no treatment interventions known to be effective for management of over dosage of MM-398.

In some embodiments, methods of administering MM-398 comprise advising patients of one or more risks selected from the group consisting of:
Hypersensitivity to irinotecan HCl or MM-398: Advise patients of the potential risk of severe hypersensitivity and that MM-398 is contraindicated in patients with a history of severe allergic reactions with irinotecan HCl or MM-398. Instruct patients to seek immediate medical attention for signs of severe hypersensitivity reaction such as chest tightness; shortness of breath; wheezing; dizziness or faintness; or swelling of the face, eyelids, or lips;
Severe Neutropenia: Advise patients of the risk of neutropenia leading to severe and life threatening infections and the need for monitoring of blood counts. Instruct patients to contact their healthcare provider immediately if experiencing signs of infection, such as fever, chills, dizziness, or shortness of breath (See “WARNINGS AND PRECAUTIONS: Severe Neutropenia”).
Severe Diarrhea: Inform patients of the risk of severe diarrhea. Advise patients to contact their healthcare provider if they experience persistent vomiting or diarrhea; black or bloody stools; or symptoms of dehydration such as lightheadedness, dizziness, or faintness;
Interstitial lung disease: Inform patients of the potential risk of ILD. Advise patients to contact their healthcare provider as soon as possible for new onset cough or dyspnea;
Cholinergic reactions: Inform patients of the risk of cholinergic reactions occurring within 24 hours of MM-398 administration. Advise patients to contact their healthcare provider if they experience rhinitis, increased salivation, flushing, bradycardia, miosis, lacrimation, diaphoresis, and intestinal hyperperistalsis with abdominal cramping and early onset diarrhea;
Embryofetal toxicity: Inform females of reproductive potential of the potential risk to a fetus, to use effective contraception during treatment and for one month after the final dose, and to inform their healthcare provider of a known or suspected pregnancy;
Contraception: Advise male patients with female partners of reproductive potential to use condoms during treatment with MM-398 and for four months after the final dose
Lactation: Advise women not to breastfeed during treatment with MM-398 and for one month after the final dose.

In some embodiments, MM-398 is not administered to patients with severe hypersensitivity reaction to MM-398 or irinotecan HCl, patients with hypersensitivity reaction to MM-398 or to certain female patients of reproductive potential or of the potential risk to a fetus, or to female patients who breast feed.

Following administration of non-liposomal irinotecan (i.e., irinotecan HCl), exposure to irinotecan or its active metabolite, SN-38, is substantially reduced in adult and pediatric patients concomitantly receiving the CYP3A4 enzyme-inducing anticonvulsants phenytoin, phenobarbital, carbamazepine, or St. John's wort. Avoid the use of strong CYP3A4 inducers (e.g., rifampin, phenytoin, carbamazepine, rifabutin, rifapentin, phenobarbital, and St. John's wort) if possible. Substitute non-enzyme inducing therapies at least 2 weeks prior to initiation of MM-398 therapy.

In some embodiments, MM-398 is not administered to patients receiving strong CYP3A4 Inducers. Following administration of non-liposomal irinotecan (i.e., irinotecan HCl), patients receiving concomitant ketoconazole, a CYP3A4 and UGT1A1 inhibitor, have increased exposure to irinotecan and its active metabolite SN-38. Co-administration of MM-398 with other inhibitors of CYP3A4 (e.g., clarithromycin, indinavir, itraconazole, lopinavir, nefazodone, nelfinavir, ritonavir, saquinavir, telaprevir, voriconazole) or UGT1A1 (e.g., atazanavir, gemfibrozil, indinavir) may increase systemic exposure to irinotecan or SN-38. Avoid the use of strong CYP3A4 or UGT1A1 inhibitors if possible. Discontinue strong CYP3A4 inhibitors at least 1 week prior to starting MM-398 therapy. In some embodiments, MM-398 is administered to a patient who has received a strong CYP3A inducers, by substituting the strong CYP3A inducer with non-enzyme inducing therapies at least 2 weeks prior to initiation of MM-398. In some embodiments, MM-398 is not administered to patients receiving strong CYP3A4 inhibitors. In some embodiments, MM-398 is administered to a patient who has received a strong CYP3A4 inhibitor, by substituting the strong CYP3A4 inhibitor with non-enzyme inducing therapies at least 1 week prior to initiation of MM-398. Do not administer MM-398 to patients with bowel obstruction. Withhold MM-398 for diarrhea of Grade 2-4 severity. Administer loperamide for late diarrhea of any severity. Administer atropine for early diarrhea of any severity.

No studies have been performed to assess the potential of irinotecan liposome for carcinogenicity, genotoxicity or impairment of fertility. Intravenous administration of irinotecan hydrochloride to rats once weekly for 13 weeks followed by a 91 week recovery period resulted in a significant linear trend between irinotecan HCl dosage and the incidence of combined uterine horn endometrial stromal polyps and endometrial stromal sarcomas. Irinotecan HCl was clastogenic both in vitro (chromosome aberrations in Chinese hamster ovary cells) and in vivo (micronucleus test in mice). Neither irinotecan nor its active metabolite, SN-38, was mutagenic in the in vitro Ames assay.

Dedicated fertility studies have not been performed with irinotecan liposome injection. Atrophy of male and female reproductive organs was observed in dogs receiving irinotecan liposome injection every 3 weeks at doses equal to or greater than 13 mg/kg, (approximately 3 times the clinical exposure of irinotecan following administration to MM-398 dosed at 70 mg/m²) for a total of 6 doses. No significant adverse effects on fertility and general reproductive performance were observed after intravenous administration of irinotecan HCl in doses of up to 6 mg/kg/day to rats; however, atrophy of male reproductive organs was observed after multiple daily irinotecan HCl doses both in rodents at 20 mg/kg (approximately 0.007 times the clinical irinotecan exposure following MM-398 administration at 70 mg/m²) and in dogs at 0.4 mg/kg (0.0005 times the clinical exposure to irinotecan following administration of MM-398).

### Example 1: NAPOLI-1 Clinical Trial

NAPOLI-1 was an international randomized human Phase 3 clinical trial evaluating the use of the irinotecan liposome MM-398 in patients with a diagnosis of metastatic pancreatic cancer patients previously treated with a gemcitabine based therapy. The NAPOLI-1 trial is summarized below.

NAPOLI-1 was an open label, randomized, stratified by albumin (<4.0 g/dL vs ≧4.0 g/dL), Karnofsky Performance Status (KPS) (70 & 80 vs ≧90), and ethnicity (Caucasian vs East Asian vs others). The primary analysis compared each treatment arm to its corresponding 5-FU/LV control for OS by unstratified log-rank test; family-wise type I error rate was controlled at the 2-sided 0.05 level using the Bonferroni-Holm method. Primary analysis planned when at least 305 death events occurred to have 85% power to detect HR=0.67 in the MM-398 arm and 98% power to detect HR=0.50 in the MM-398+5-FU/LV arm. A supportive stratified analysis, accounting for the randomization strata, was performed.

The primary endpoint of the NAPOLI-1 study was overall survival; and the key secondary endpoints were Progression Free Survival (PFS), Objective Response Rate (ORR), Tumor Marker Response (CA 19-9) and Safety. The study was amended to add the MM-398+5-FU/LV arm once safety data on the combination became available. Only those patients enrolled in the 5FU/LV arm after the amendment (N=119), were used as the control for the combination arm.

Key Inclusion Criteria for the NAPOLI-1 trial were: Adenocarcinoma of the exocrine pancreas; Metastatic disease, measurable or non-measurable; Progressed after prior gemcitabine or gemcitabine-containing therapy; KPS≧70; Adequate bone marrow, hepatic (bilirubin within normal range for the institution and albumin≧3 g/dL), and renal function.

Sixty six PP patient were in the MM398+5-FU/LV arm and 71 PP patients were in the 5-FU/LV arm of NAPOLI-1. The NAPOLI-1 study was well balanced. Patients in the MM-398+5FU/LV and 5FU/LV arms were consistent across the following patient characteristics: prognostic factors, demographics (age, sex, race), tumor and pre and post treatment characteristics. Post-study anticancer therapy was 31% in the MM-398+5-FU/LV arm and 38% in the 5-FU/LV arm.

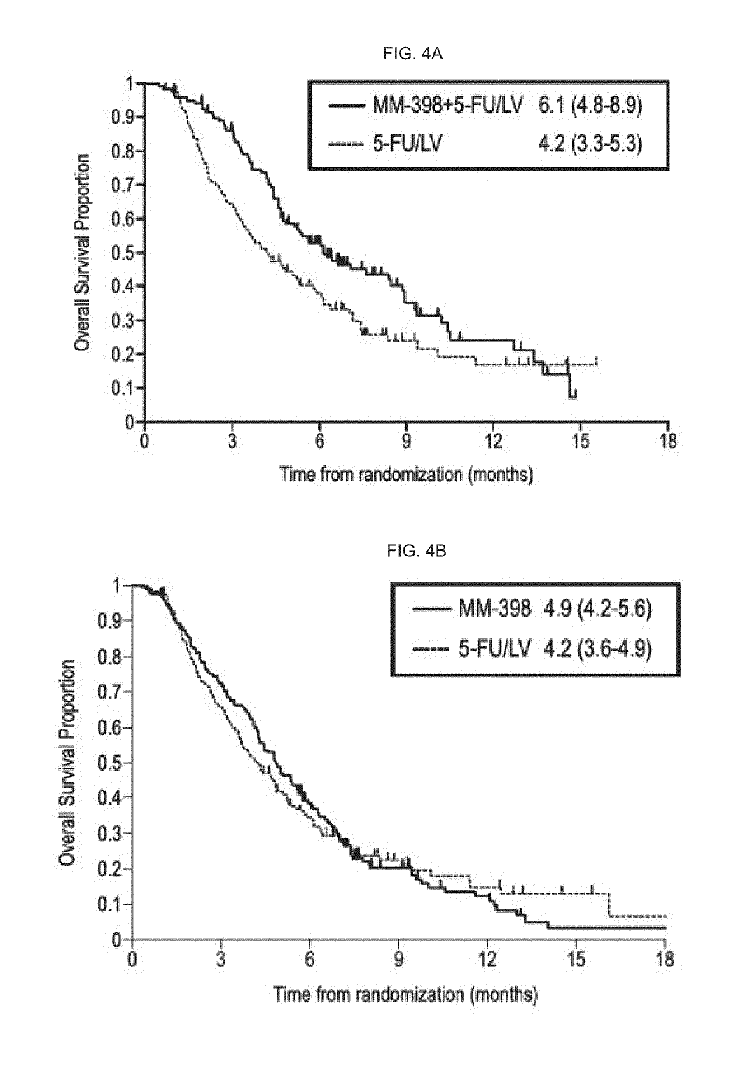

Overall Survival Results from the ITT patient group in the NAPOLI-1 clinical trial are shown in FIGS. 4A and 4B. FIG. 4A shows the median overall survival rate for the MM-398+5-FU/LV arm was 6.1 months (95% CI 4.8-8.9) and the 5-Fu/LV arm overall survival rate was 4.2 months (95% CI 3.3-5.3) and the stratified HR was 0.57 (95 CI 0.41-0.8), p=0.0009. FIG. 4B shows the median overall survival rate for the MM-398 arm was 4.9 months (95% CI 4.2-5.6) and the 5-Fu/LV arm median OS was 4.2 months (95% CI 3.6-4.9) and the stratified HR was 0.93 (95 CI 0.71-1.21), p=035545.

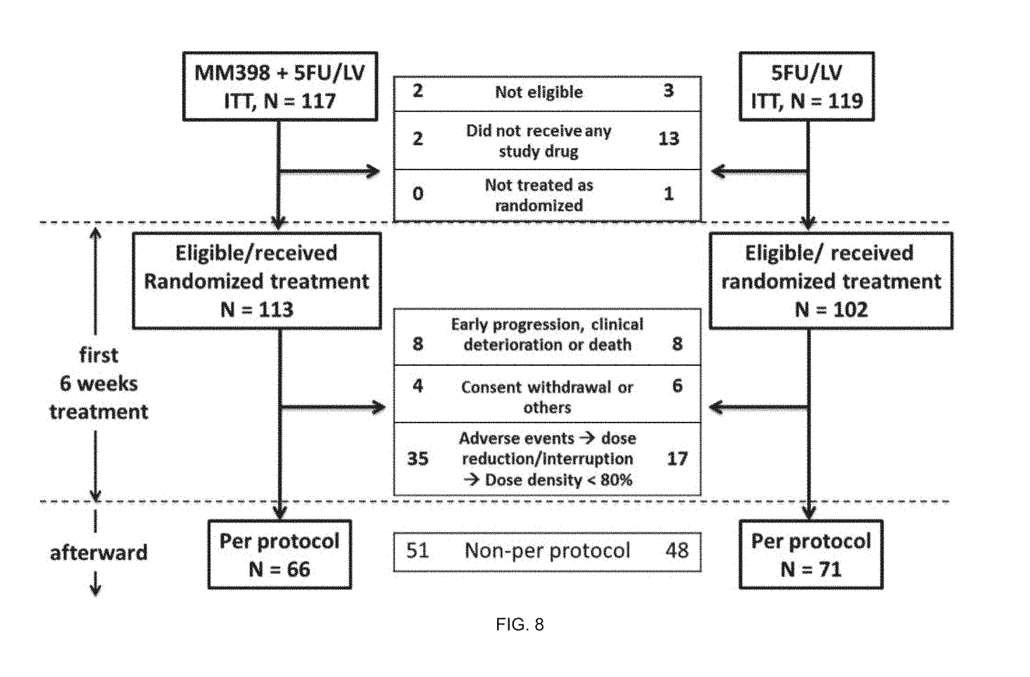

Analysis of the ITT (intent to treat) patient group demonstrated statistically significant increase in overall survival (OS) of MM-398+5-FU/LV (MM-398 80 mg/m² q2w regimen) over 5-FU/LV alone (FIGS. 4A and 4B) of 6.1 months versus 4.2 months respectively. FIG. 8 is a flow chart explaining the Intent To Treat (ITT) patients (all randomized patients) Non-per protocol (NPP) and PP (Per Protocol) populations. The Per Protocol population comprises eligible patients who received≧80% dose density of the protocol defined treatment during the first 6 weeks of treatment and did not have the following protocol violations: receipt of any prohibited therapies as defined in the protocol, not receiving treatment as randomized, or inclusion/exclusion criteria deviations.

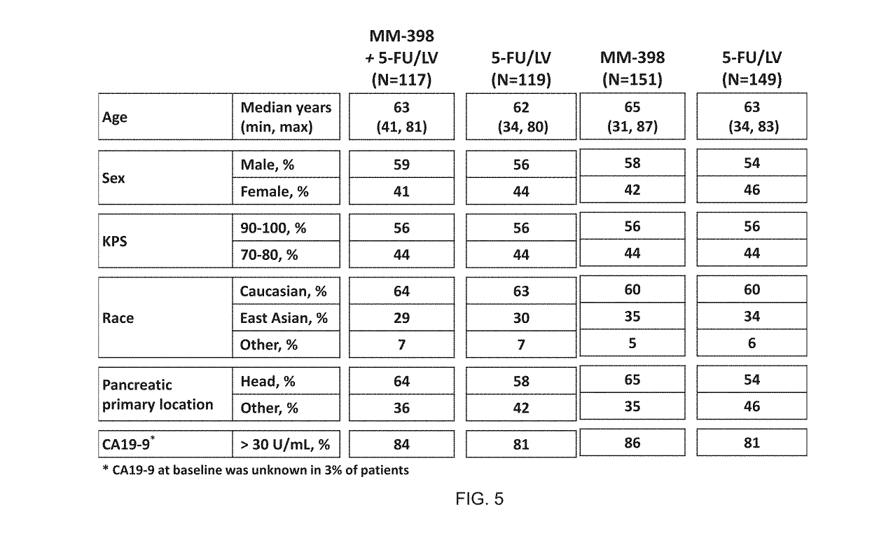

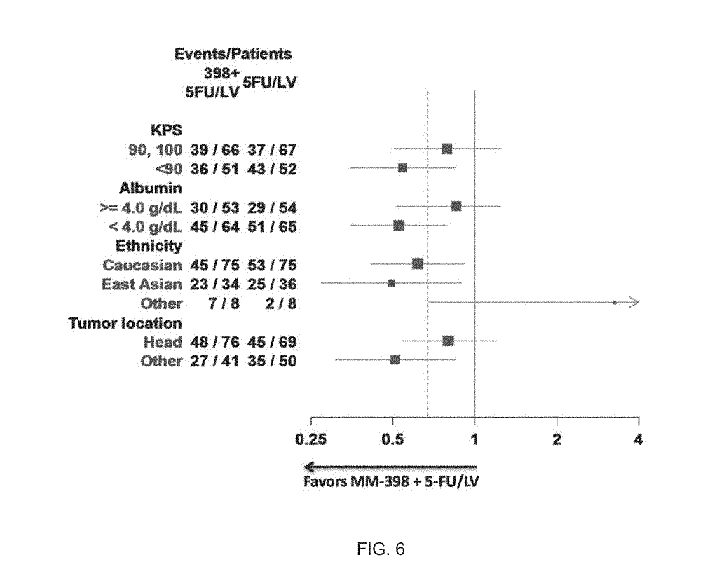

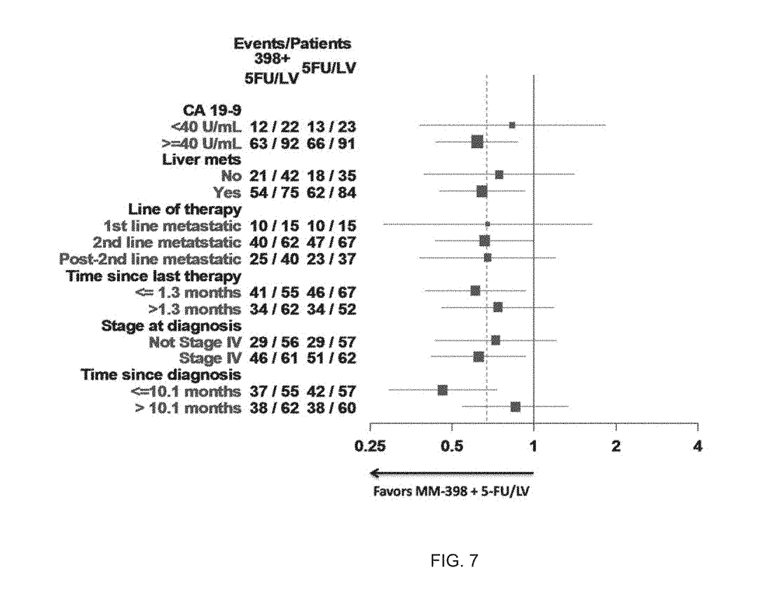

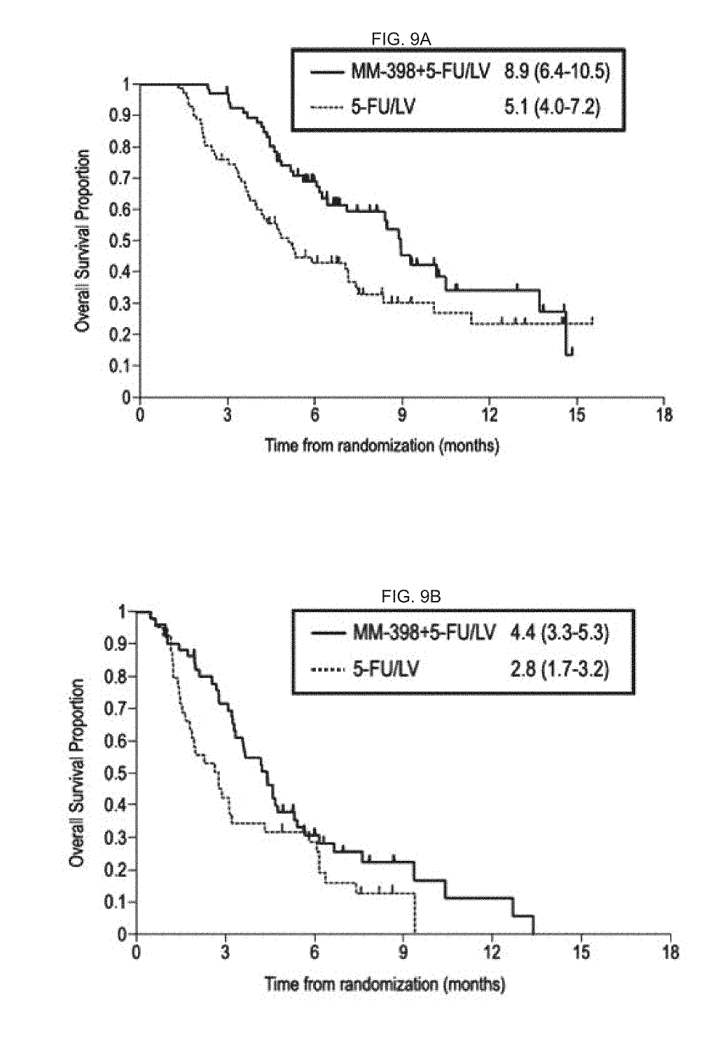

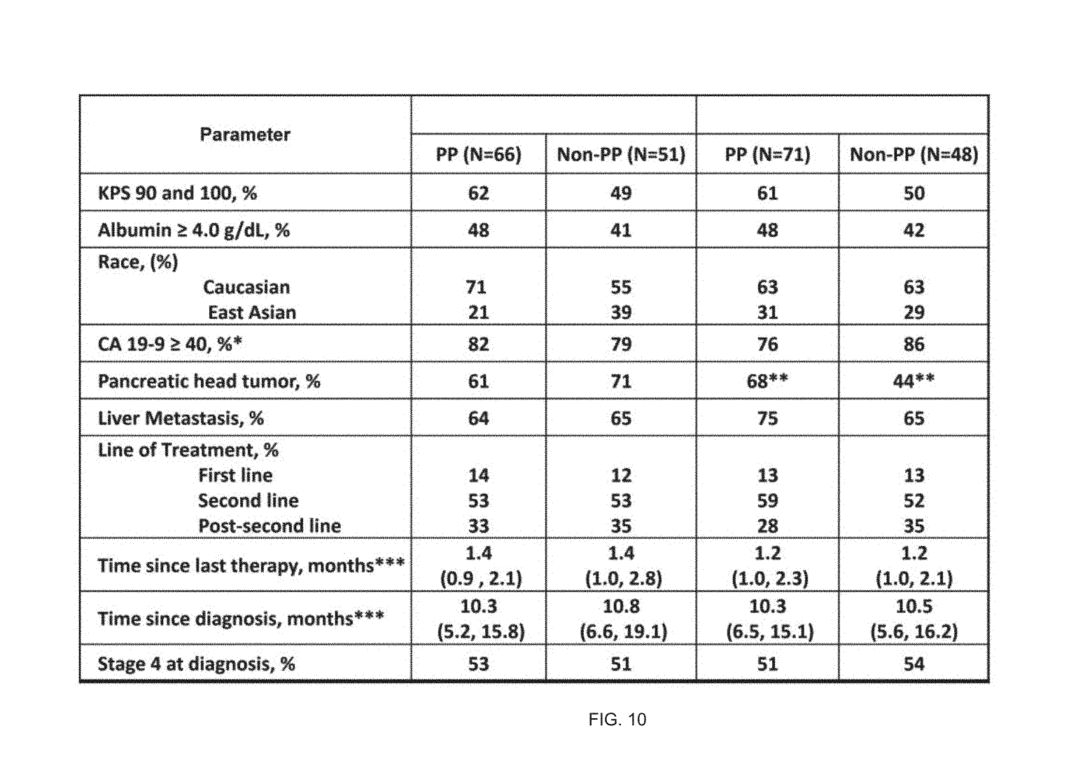

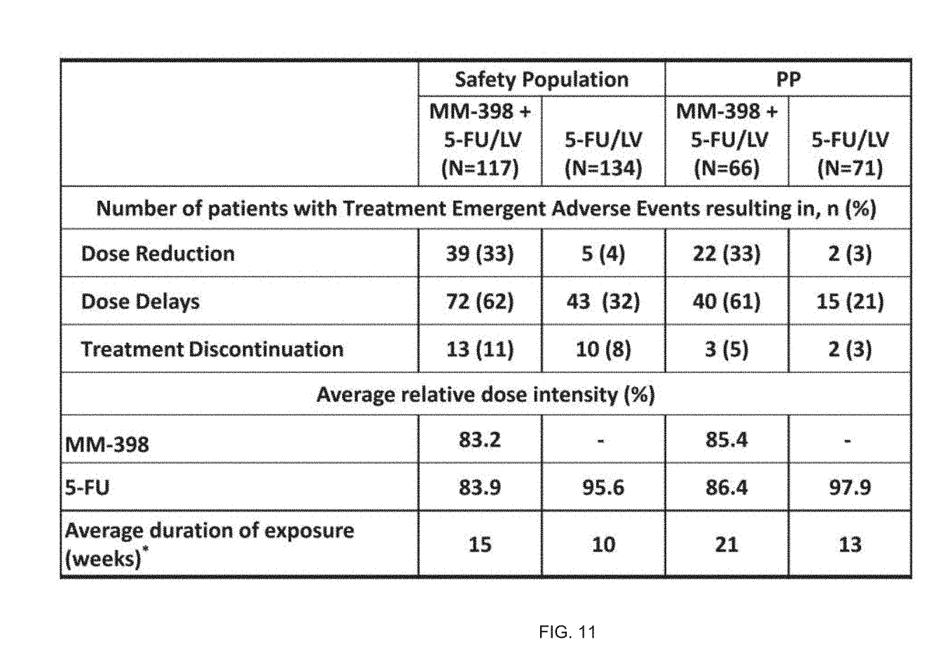

Significant increase in OS was also observed in Progression Free Survival (PFS), Objective Response Rate (ORR), and Tumor Marker Response (CA 19-9) response (FIG. 5). In comparison, MM-398, as a single agent (120 mg/m² q3w regimen), did not show a significant difference in OS. In addition, a Forest plot sensitivity analyses of the NAPOLI-1 human clinical trial favored MM-398+5-FU/LV over 5-FU/LV across prognostic subgroups, tumor characteristics and previous treatment (FIGS. 6 and 7). Notably, the Per Protocol (PP) population (described in FIG. 8) of patients in the NAPOLI-1 human clinical trial (patients receiving 6 weeks of treatment), the MM-398+5-FU/LV combination regimen achieved a median OS of 8.9 months versus 5.1 months in the 5-FU/LV arm (stratified Hazard Ratio (HR): 0.47, p=0.0018; FIGS. 9A and 9B).

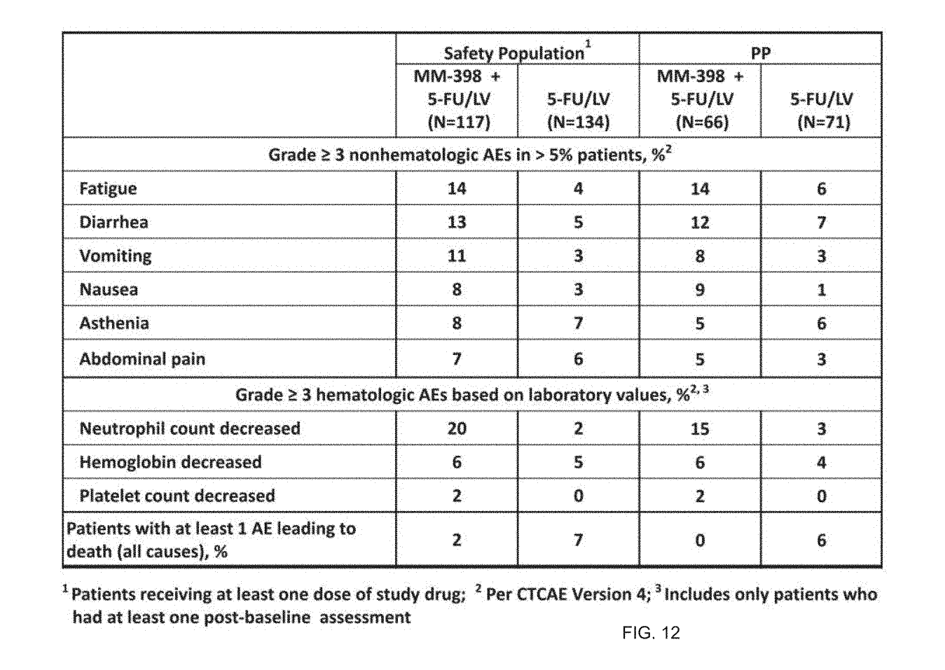

Safety profile was manageable, with most frequent being grade≧3 adverse events including neutropenia, fatigue and GI effects, such as diarrhea and vomiting (FIG. 12).

Overall Survival Results for the PP and Non-PP populations are shown in FIGS. 9A and 9B respectively. FIG. 9A shows the median overall survival rate for the PP population; the median OS for the MM-398+5-FU/LV arm was 8.9 months (95% CI 6.4-10.5) and the median OS rate for the 5-Fu/LV arm was 5.1 months (95% CI 4.0-7.2) and the stratified HR was 0.47 (95 CI 0.29-0.77), p=0.0018. FIG. 9B shows the median overall survival rate for the Non-PP population; the median OS for the MM-398+5-FU/LV arm was 4.4 months (95% CI 3.3-5.3) and the median OS rate for the 5-Fu/LV arm was 2.8 months (95% CI 1.7-3.2) and the stratified HR was 0.56 (95 CI 0.33-0.97), p=0.365.

As used herein, doses requiring 200 mg/m² leucovorin are to be understood to require 200 mg/m² of the (1) enantiomer of leucovorin, and doses requiring 400 mg/m² leucovorin are to be understood to require 400 mg/m² of the (l+d) racemate of leucovorin. It is further understood that a dose having 200 mg/m² of the (1) enantiomer of leucovorin, and a dose having 400 mg/m² of the (l+d) racemate of leucovorin contain equivalent amounts of the pharmaceutically active (1) form of leucovorin.

The efficacy of MM-398 was evaluated in Study 1, a three-arm, randomized, open-label trial in patients with metastatic pancreatic adenocarcinoma with documented disease progression, after gemcitabine or gemcitabine-based therapy. Key eligibility criteria included Karnofsky Performance Status (KPS)≧70, serum bilirubin within institution limits of normal, and albumin≧3.0 g/dL. Patients were randomized to receive MM-398 plus fluorouracil/leucovorin (MM-398/5-FU/LV), MM-398, or fluorouracil/leucovorin (5-FU/LV). Randomization was stratified by ethnicity (White vs. East Asian vs. other), KPS (70-80 vs. 90-100), and baseline albumin level (≧4 g/dL vs. 3.0-3.9 g/dL). Patients randomized to MM-398/5-FU/LV received MM-398 70 mg/m² as an intravenous infusion over 90 minutes, followed by leucovorin 400 mg/m² intravenously over 30 minutes, followed by fluorouracil 2400 mg/m² intravenously over 46 hours, every 2 weeks. The MM-398 dose in Study 1, 70 mg/m², is based on irinotecan as the free base (equivalent to 80 mg/m² of irinotecan as the hydrochloride trihydrate).

Patients randomized to MM-398 as a single agent received MM-398 100 mg/m² as an intravenous infusion over 90 minutes every 3 weeks. Patients randomized to 5-FU/LV received leucovorin 200 mg/m² intravenously over 30 minutes, followed by fluorouracil 2000 mg/m² intravenously over 24 hours, administered on Days 1, 8, 15 and 22 of a 6-week cycle. Patients homozygous for the UGT1A1*28 allele initiated MM-398 at a reduced dose (50 mg/m² MM-398, if given with 5-FU/LV or 70 mg/m² MM-398 as a single agent). When MM-398 was withheld or discontinued for adverse reactions, 5-FU was also withheld or discontinued. When the dose of MM-398 was reduced for adverse reactions, the dose of 5-FU was reduced by 25%. Treatment continued until disease progression or unacceptable toxicity.

The major efficacy outcome measure was overall survival (OS) with two pairwise comparisons: MM-398 versus 5-FU/LV and MM-398/5-FU/LV versus 5-FU/LV. Additional efficacy outcome measures were progression-free survival (PFS) and objective response rate (ORR). Tumor status assessments were conducted at baseline and every 6 weeks thereafter. The trial was initiated as a two-arm study and amended after initiation to include a third arm (MM-398/5-FU/LV). The comparisons between the MM-398/5-FU/LV and the 5-FU/LV arms are limited to patients enrolled in the 5-FU/LV arm after this protocol amendment.

Four hundred seventeen patients were randomized to: MM-398/5-FU/LV (N=117), MM-398 (N=151), or 5-FU/LV (N=149). Baseline demographics and tumor characteristics for the 236 patients randomized to MM-398/5-FU/LV or 5-FU/LV (N=119) after the addition of the third arm to the study were a median age of 63 years (range 34-81 years) and with 41%≧65 years of age; 58% were men; 63% were White, 30% were Asian, 3% were Black or African American, and 5% were other. Mean baseline albumin level was 3.97 g/dL, and baseline KPS was 90-100 in 53% of patients. Disease characteristics included liver metastasis (67%) and lung metastasis (31%). A total of 13% of patients received gemcitabine in the neoadjuvant/adjuvant setting only, 55% of patients had 1 prior line of therapy for metastatic disease, and 33% of patients had 2 or more prior lines of therapy for metastatic disease. All patients received prior gemcitabine (alone or in combination with another agent); 54% received prior gemcitabine in combination with another agent, and 13% received prior gemcitabine in combination with nab-paclitaxel.

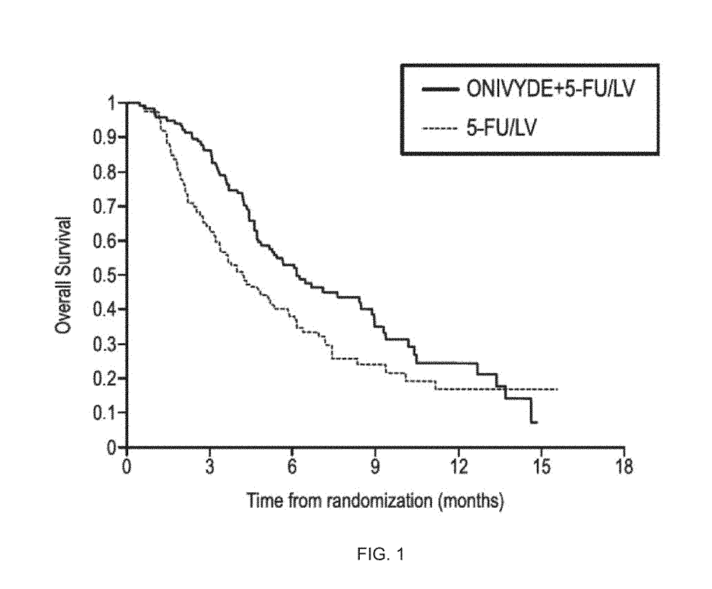

Study 1 demonstrated a statistically significant improvement in overall survival for the MM-398/5-FU/LV arm over the 5-FU/LV arm as summarized in Table 5 and shown graphically in FIG. 1.

There was no improvement in overall survival for the MM-398 arm over the 5-FU/LV arm (hazard ratio=1.00, p-value=0.97 (two-sided log-rank test)).

TABLE 5
Efficacy Results from Study 1†‡

| | |
|---|---|
| MM-398/5-FU/LV | 5-FU/LV |
| (N = 117) | (N = 119) |

Overall Survival

| | | |
|---|---|---|
| Number of Deaths, n (%) | 77 (66) | 86 (72) |
| Median Overall Survival (months) | 6.1 | 4.2 |
| (95% CI) | (4.8, 8.5) | (3.3, 5.3) |

| | |
|---|---|
| Hazard Ratio (95% CI) | 0.68 (0.50, 0.93) |
| p-value (log-rank test) | 0.014 |

Progression-Free Survival

| | | |
|---|---|---|
| Death or Progression, n (%) | 83 (71) | 94 (79) |
| Median Progression-Free Survival | 3.1 | 1.5 |
| (months) | | |
| (95% CI) | (2.7, 4.2) | (1.4, 1.8) |

| | |
|---|---|
| Hazard Ratio (95% CI) | 0.55 (0.41, 0.75) |
| p-value (log rank test) | p < 0.001 |

Objective Response Rate

| | | |
|---|---|---|
| Confirmed complete or partial | 9 (7.7) | 1 (0.8) |
| response n (%) | | |
| (95% CI) | (2.9, 12.5) | (0, 2.5) |

†5-FU/LV = 5-fluorouracil/leucovorin; CI = confidence interval
‡The MM-398 dose in Study 1, 70 mg/m², is based on irinotecan as the free base (equivalent to 80 mg/m² of irinotecan as a hydrochloride trihydrate).

Table 5 sets out that the medial overall survival of the MM-398/5-FU/LV is 6.1 months. Median overall survival is used to express survival rates. It is the amount of time after which, in the MM-398/5-FU/LV of Study 1 described herein (presented in table 5), 50% of the patients have died and 50% have survived in a study population. The expected lifetime in months from commencement of treatment with the MM-398/5-FU/LV treatment regimen as disclosed herein is defined by the parameter, tₛᵤᵣᵥ. In some embodiments, the tₛᵤᵣᵥ of an individual being treated is at least ⅔ of the median overall survival rate (≧4.1 months (to one decimal place (dp))), such as at least ⅚ of the median overall survival (≧5.1 months (1 dp)) or at least the median overall survival (≧6.1 months). In some embodiments, the tₛᵤᵣᵥ of an individual being treated is less than 2 times the median overall survival rate (<12.2 months (1 dp)), such as less than 1.5 times the median overall survival (<9.15 months (2 dp)) or less than 1.2 times the median overall survival (<7.32 months (1 dp)). In some embodiments, the tₛᵤᵣᵥ of an individual being treated is at least ⅔ of the median overall survival rate and less than 2 times the median overall survival rate, such as less than 1.5 times the median overall survival or less than 1.2 times the median overall survival. In some embodiments, the tₛᵤᵣᵥ of an individual being treated is at least ⅚ of the median overall survival rate and less than 2 times the median overall survival rate, such as less than 1.5 times the median overall survival or less than 1.2 times the median overall survival. In some embodiments, the tₛᵤᵣᵥ of an individual being treated is at least the median overall survival rate and less than 2 times the median overall survival rate, such as less than 1.5 times the median overall survival or less than 1.2 times the median overall survival.

Because clinical trials are conducted under widely varying conditions, adverse reaction rates observed in clinical trials of MM-398 cannot be directly compared to rates in clinical trials of other drugs and may not reflect the rates observed in practice.

The safety data described below are derived from Study 1, an international, randomized, active-controlled, open-label trial, in which patients with metastatic adenocarcinoma of the pancreas previously treated with gemcitabine-based therapy received any part of protocol-specified therapy, as follows: MM-398 70 mg/m² with leucovorin 400 mg/m² and fluorouracil 2400 mg/m² over 46 hours every 2 weeks (MM-398/5-FU/LV; n=117), MM-398 100 mg/m² every 3 weeks (n=147), or leucovorin 200 mg/m² and fluorouracil 2000 mg/m² over 24 hours weekly for 4 weeks followed by 2 week rest (5-FU/LV; n=134). Serum bilirubin within the institutional normal range, albumin≧3 g/dL, and Karnofsky Performance Status (KPS)≧70 were required for study entry. The median duration of exposure was 9 weeks in the MM-398/5-FU/LV arm, 9 weeks in the MM-398 monotherapy arm, and 6 weeks in the 5-FU/LV arm.

The most common adverse reactions (≧20%) of MM-398 were diarrhea, fatigue/asthenia, vomiting, nausea, decreased appetite, stomatitis, and pyrexia. The most common laboratory abnormalities (≧10% Grade 3 or 4) were lymphopenia and neutropenia. The most common serious adverse reactions (≧2%) of MM-398 were vomiting, diarrhea, neutropenic fever or sepsis, nausea, pyrexia, anemia, pneumonia, sepsis, dehydration, septic shock, acute renal failure, thrombocytopenia and ileus.

The most common adverse reactions (≧20%) of patients receiving MM-398 were diarrhea, fatigue/asthenia, vomiting, nausea, decreased appetite, stomatitis, and pyrexia. Severe (21%) or life-threatening (7%) neutropenia or neutropenic sepsis, of which 1% resulted in septic shock, occurred in patients receiving MM-398 in combination with fluorouracil and leucovorin. In some embodiments, MM-398 is withheld from patients with an absolute neutrophil count below 1500/mm³ or neutropenic fever. The blood cell counts of patients are preferably monitored periodically during treatment. Severe or life-threatening diarrhea occurred in 13% of patients receiving MM-398 in combination with 5FU and leucovorin.

Severe or life threatening neutropenia occurred in 27% of patients receiving MM-398/5-FU/LV compared to 2% of patients receiving fluorouracil/leucovorin alone (5-FU/LV) in Study 1 described herein. The expected likelihood of severe or life threatening neutropenia (as a %) during treatment with MM-398/5-FU/LV in the protocol used in Study 1, is expressed by the parameter Pₐₑₙ. In some embodiments, Pₐₑₙ is <50%. In some embodiments it is <45%, such as <40%, <35%, <30% or ≦27%.

In Study 1, Grade 3 or 4 diarrhea occurred in 13% receiving MM-398/5-FU/LV compared to 4% receiving 5-FU/LV. The expected likelihood of Grade 3 or 4 diarrhea (as a %) during treatment with MM-398/5-FU/LV in the protocol used in Study 1, is expressed by the parameter P_{aed}. In some embodiments, P_{aed} is <50%. In some embodiments it is <45%, such as <40%, <35%, <30%, <25%, <20%, <15% or ≦13%.

In some embodiments, tₛₑᵣᵥ is ≧6.1 months, Pₐₑₙ is ≦27% and P_{aed} is ≦13%.

Adverse reactions led to permanent discontinuation of MM-398 in 11% of patients receiving MM-398/5-FU/LV; the most frequent adverse reactions resulting in discontinuation of MM-398 were diarrhea, vomiting, and sepsis. Dose reductions of MM-398 for adverse reactions occurred in 33% of patients receiving MM-398/5-FU/LV; the most frequent adverse reactions requiring dose reductions were neutropenia, diarrhea, nausea, and anemia. MM-398 was withheld or delayed for adverse reactions in 62% of patients receiving MM-398/5-FU/LV; the most frequent adverse reactions requiring interruption or delays were neutropenia, diarrhea, fatigue, vomiting, and thrombocytopenia.

Table 6 provides the frequency and severity of adverse reactions in Study 1 that occurred with higher incidence (≧5% difference for Grades 1-4 or ≧2% difference for Grades 3-4) in patients who received MM-398/5 FU/LV compared to patients who received 5-FU/LV arm.

TABLE 6
Adverse Reactions with Higher Incidence (≧5% Difference
for Grades 1-4* or ≧2% Difference for Grades 3
and 4) in the MM-398/5-FU/LV.

| | MM-398/5-FU/LV | | 5-FU/LV | |
|---|---|---|---|---|
| | N = 117 | | N = 134 | |
| | Grades | Grades | Grades | Grades |
| | 1-4 | 3-4 | 1-4 | 3-4 |
| Adverse Reaction | (%) | (%) | (%) | (%) |

Gastrointestinal disorders

| | | | | |
|---|---|---|---|---|
| Diarrhea | 59 | 13 | 26 | 4 |
| Diarrhea, early^{†} | 30 | 3 | 15 | 0 |
| Diarrhea, late^{‡} | 43 | 9 | 17 | 4 |
| Vomiting | 52 | 11 | 26 | 3 |
| Nausea | 51 | 8 | 34 | 4 |
| Stomatitis^{§} | 32 | 4 | 12 | 1 |

Infections and infestations

| | | | | |
|---|---|---|---|---|
| Sepsis | 4 | 3 | 2 | 1 |
| Neutropenic fever/ | 3 | 3 | 1 | 0 |
| neutropenic sepsis [symbol] | | | | |
| Gastroenteritis | 3 | 3 | 0 | 0 |
| Device related | 3 | 3 | 0 | 0 |
| infection | | | | |

General disorders and administration site conditions

| | | | | |
|---|---|---|---|---|
| Fatigue/asthenia | 56 | 21 | 43 | 10 |
| Pyrexia | 23 | 2 | 11 | 1 |

Metabolism and nutrition disorders

| | | | | |
|---|---|---|---|---|
| Decreased appetite | 44 | 4 | 32 | 2 |
| Weight loss | 17 | 2 | 7 | 0 |
| Dehydration | 8 | 4 | 7 | 2 |

Skin and subcutaneous tissue disorders

| | | | | |
|---|---|---|---|---|
| Alopecia | 14 | 1 | 5 | 0 |

*NCI CTCAE v4.0
^{†}Early diarrhea: onset within 24 hours of MM-398 administration
^{‡}Late diarrhea: onset >1 day after MM-398 administration
^{§}Includes stomatitis, aphthous stomatitis, mouth ulceration, mucosal inflammation. [symbol]
Includes febrile neutropenia.

Cholinergic Reactions:

MM-398 can cause cholinergic reactions manifesting as rhinitis, increased salivation, flushing, bradycardia, miosis, lacrimation, diaphoresis, and intestinal hyperperistalsis with abdominal cramping and early onset diarrhea. In Study 1, Grade 1 or 2 cholinergic symptoms other than early diarrhea occurred in 12 (4.5%) MM-398-treated patients. Six of these 12 patients received atropine; in 1 of the 6 patients who received atropine, the atropine was administered for cholinergic symptoms other than diarrhea.

Infusion Reactions:

Infusion reactions, consisting of allergic reaction, rash, urticaria, periorbital edema, or pruritus, occurring on the day of MM-398 administration were reported in 3% of patients receiving MM-398 or MM-398/5-FU/LV.

Additional clinically significant adverse reactions occurring in <10% of MM-398/5-FU/LV-treated patients were:

Cardiovascular:

Severe hypotension

Laboratory abnormalities that occurred with higher incidence in the MM-398/5 FU/LV arm compared to the 5-FU/LV arm (≧5% difference) are summarized in the following table 7.

TABLE 7
Laboratory Abnormalities with Higher Incidence (≧5%
Difference) in the MM-398/5-FU/LV Arm*#

| | MM-398/5-FU/LV | | 5-FU/LV | |
|---|---|---|---|---|
| | Grades | Grades | Grades | Grades |
| Laboratory | 1-4 | 3-4 | 1-4 | 3-4 |
| abnormality | (%) | (%) | (%) | (%) |

Hematology

| | | | | |
|---|---|---|---|---|
| Anemia | 97 | 6 | 86 | 5 |
| Decreased lymphocytes | 81 | 27 | 75 | 17 |
| Decreased leukocytes | 67 | 16 | 20 | 0 |
| Decreased neutrophil | 52 | 20 | 6 | 2 |
| counts | | | | |
| Decreased platelet | 41 | 2 | 33 | 0 |
| counts | | | | |

Hepatic

| | | | | |
|---|---|---|---|---|
| Increased alanine | 51 | 6 | 37 | 1 |
| aminotransferase | | | | |
| (ALT) | | | | |
| Decreased albumin | 43 | 2 | 30 | 0 |

Metabolic

| | | | | |
|---|---|---|---|---|
| Decreased magnesium | 35 | 0 | 21 | 0 |
| Decreased potassium | 32 | 2 | 19 | 2 |
| Decreased calcium | 32 | 1 | 20 | 0 |
| Decreased phosphate | 29 | 4 | 18 | 1 |
| Decreased sodium | 27 | 5 | 12 | 3 |

Renal

| | | | | |
|---|---|---|---|---|
| Increased creatinine | 18 | 0 | 13 | 0 |

*NCI CTCAE v4.0, worst grade shown
#Percent based on number of patients with a baseline and at least one post-baseline measurement.

Of the 264 patients who received MM-398 as a single agent or in combination with 5-FU and leucovorin in Study 1, 49% were ≧65 years old and 13% were ≧75 years old. No overall differences in safety and effectiveness were observed between these patients and younger patients.

### Example 2: Overall Survival Analysis of the Napoli-1 Phase 3 Study

NAPOLI-1 is a global, randomized Phase 3 study evaluating nal-IRI—a nanoliposomal irinotecan—with or without 5-FU/LV in 417 patients with Metastatic Pancreatic Cancer (mPAC) previously treated with gemcitabine-based therapy. Primary survival analysis was based on 313 events. Nal-IRI+5FU/LV significantly improved overall survival (OS, primary endpoint), 6.1 months (mo) vs 4.2 mo; with 5-FU/LV (unstratified hazard ratio [HR]=0.67; P=0.012). Primary endpoint was supported by improved progression-free survival, time to treatment failure, objective response and CA 19-9 tumor marker response rates, and manageable toxicities. An updated analysis of OS, 6- and 12-month-survival estimates, and safety is presented.

Methods: The updated descriptive analysis of OS, based on 378 events, includes data from all randomized patients across the 3 arms.

Results: After 378 OS events, nal-IRI+5-FU/LV (n=117) retained an OS advantage relative to 5-FU/LV (n=119): 6.2 mo (95% confidence interval [CI], 4.8-8.4) vs 4.2 mo (95% CI, 3.3-5.3) with an unstratified HR of 0.75 (P=0.0417). In contrast, there was no OS advantage with nal-IRI monotherapy (n=151) vs 5-FU/LV (n=149): 4.9 mo [95% CI, 4.2-5.6] vs 4.2 mo [95% CI, 3.6-4.9], HR=1.08; P=0.5. Six-month survival estimates were 53% (95% CI, 44-62%) for nal-IRI+5-FU/LV vs 38% (95% CI, 29-47%) for 5-FU/LV; 12-month survival estimates were 26% (95% CI, 18-35%) for nal-IRI+5-FU/LV vs 16% (95% CI, 10-24%) for 5-FU/LV. With events in nearly all patients, the OS curves converge at about 20 months with 19 patients (16.2%) surviving beyond 20 months. This is a reason for attenuation of the HR estimate and unstratified log rank p-value. The most common grade 3+adverse events occurring at a≧2% incidence in the nal-IRI-containing arms were neutropenia, diarrhea, vomiting, and fatigue.

Conclusions: In an updated analysis, the median OS benefit for nal-IRI+5FU/LV over 5-FU/LV was maintained, with a similar safety profile. Nal-IRI+5-FU/LV may be a new standard of care for patients with mPAC previously treated with gemcitabine-based therapy.

In another embodiment, the pancreatic cancer is an exocrine pancreatic cancer selected from the group consisting of acinar cell carcinoma, adenocarcinoma, adenosquamous carcinoma, giant cell tumor, intraductal papillary-mucinous neoplasm (IPMN), mucinous cystadenocarcinoma, pancreatoblastoma, serous cystadenocarcinoma, and solid and pseudopapillary tumors. Metastatic Pancreatic Cancer (mPAC) represents a significant unmet need, with approximately 80% of patients with mPAC succumbing to disease within 12 months.

In another embodiment, the liposomal formulation of irinotecan is irinotecan sucrose octasulfate salt liposome injection.

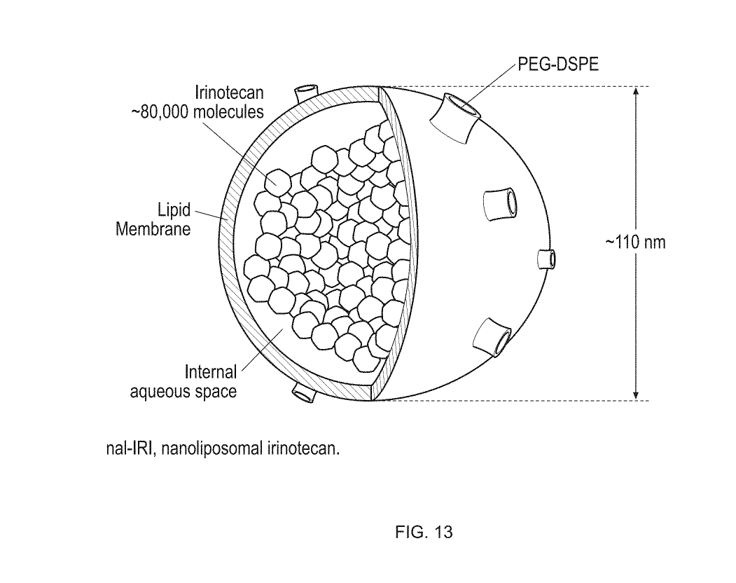

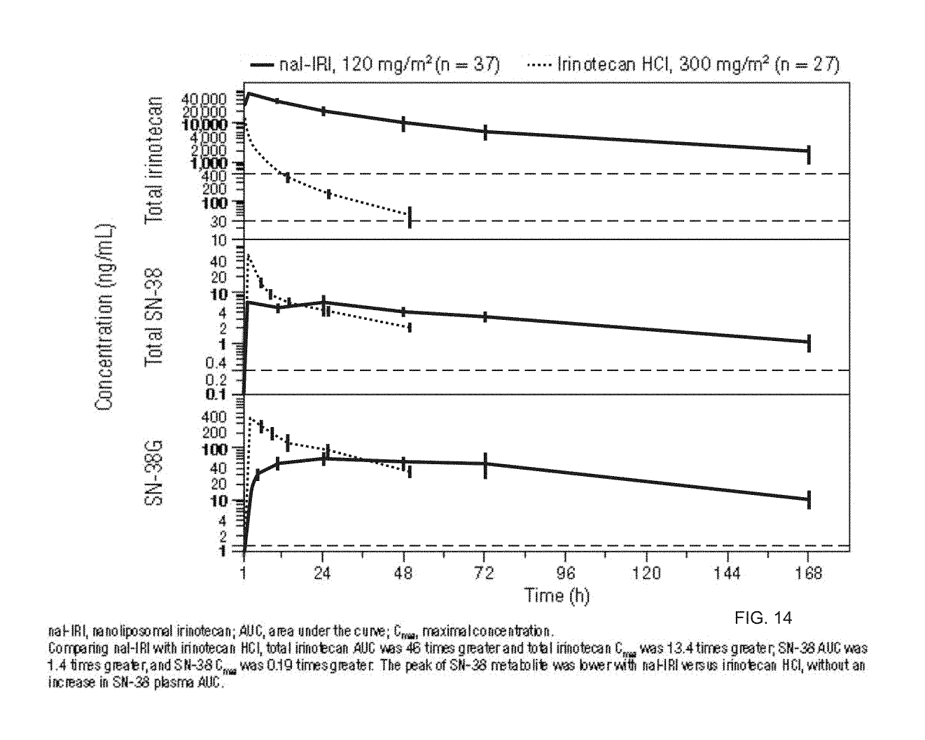

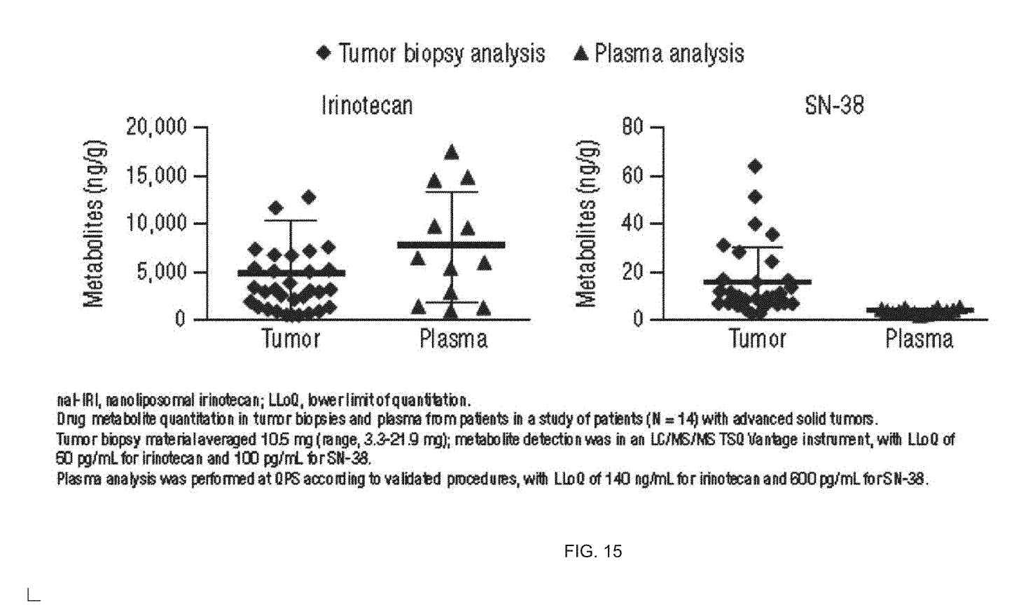

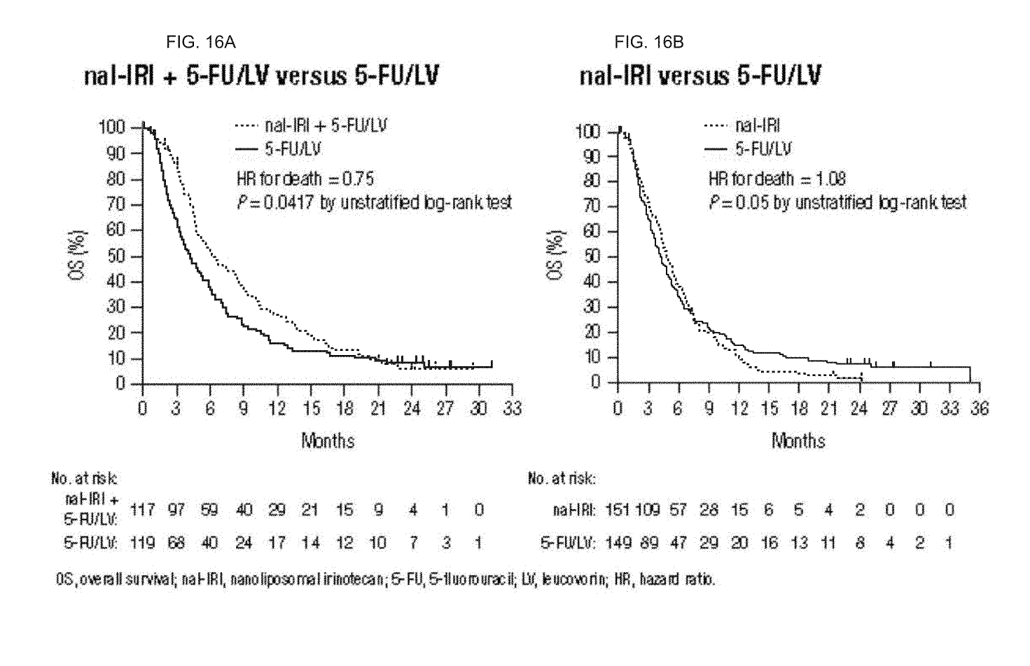

Nal-IRI (ONIVYDE® [irinotecan liposome injection]; MM-398) is a nanoliposomal formulation of irinotecan, a topoisomerase inhibitor, for intravenous use (FIG. 13). Pharmacokinetic analyses showed extended circulation of irinotecan within the liposome in patients with gastric cancer (FIG. 14). The liposome facilitates intratumoral drug deposition through the enhanced permeability and retention effect. nal-IRI promotes local activation and release of irinotecan's active metabolite, with SN-38 levels 5-fold higher in biopsied tumor tissue than in plasma at 72 hours after nal-IRI infusion (FIG. 15).

nal-IRI was recently approved by the US Food and Drug Administration for use in combination with 5-FU/LV for the treatment of patients with mPAC after disease progression following gemcitabine-based therapy, based in part on results from the primary analysis of the large (N=417) phase 3 NAPOLI-1 trial in this setting.

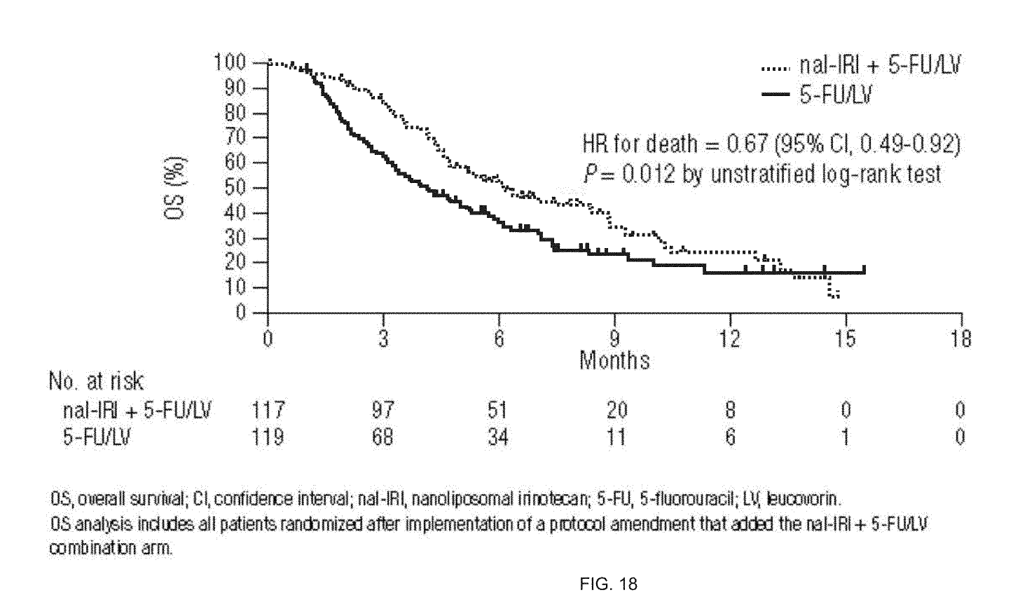

The median overall survival (OS) increased significantly with nal-IRI+5-FU/LV relative to 5-FU/LV (6.1 vs 4.2 months; unstratified hazard ratio [HR]=0.67 [95% confidence interval (CI), 0.49-0.92]; P=0.012) (FIG. 18).

The median OS did not differ between patients assigned nal-IRI monotherapy and those allocated to 5-FU/LV (4.9 vs 4.2 months; unstratified HR=0.99 [95% CI, 0.77-1.28]; P=0.94).

The median progression-free survival (PFS; 3.1 vs 1.5 months; unstratified HR=0.56 [95% CI, 0.41-0.75]; P=0.0001) and objective response rate (ORR; 16% vs 1%; P<0.0001) were also improved with nal-IRI+5-FU/LV compared with 5-FU/LV alone.

nal-IRI+5-FU/LV demonstrated a predictable and manageable safety profile; the most frequently reported grade≧3 treatment-emergent adverse events (TEAEs) were neutropenia, fatigue, diarrhea, and vomiting.

The objectives of the current descriptive analysis of the NAPOLI-1 trial are to evaluate the robustness of the previously observed OS treatment effect for nal-IRI+5-FU/LV versus 5-FU/LV control using data from longer follow-up, and to assess the long-term safety and tolerability of nal-IRI.

Methods

Study Design:

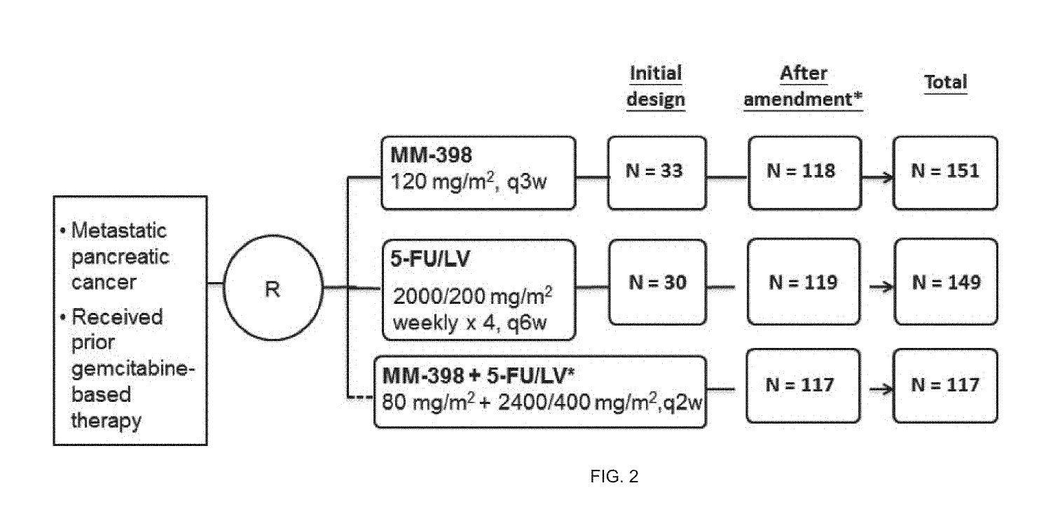

NAPOLI-1 was an international, open-label, randomized, phase 3 trial (FIG. 2). Patients were initially randomized to nal-IRI monotherapy or 5-FU/LV. The protocol was amended to add a third arm of combination nal-IRI+5-FU/LV once safety data of the combination became available from a concurrent study in metastatic colorectal cancer. The decision to add the third arm was made shortly after the trial was initiated, and 63 patients were enrolled under protocol version 1 before all sites switched to version 2. Combination therapy was compared with only those patients in the 5-FU/LV control arm who were randomized after the protocol amendment.

Randomization was stratified by baseline albumin levels, Karnofsky performance status (KPS), and ethnicity.

Analysis of the primary endpoint (OS) compared each treatment arm to its corresponding 5-FU/LV control by unstratified log-rank test; family-wise type I error rate was controlled at the 2-sided 0.05 level using the Bonferroni-Holm method. The primary analysis was planned for when ≧305 death events had occurred, in order to have 85% power to detect an HR of 0.67 in the nal-IRI arm and 98% power to detect an HR of 0.50 in the nal-IRI+5-FU/LV arm. A supportive stratified analysis that accounted for the randomization strata was also performed

Results presented herein are based on unaudited data from an updated data snapshot after 378 OS events.

Key Inclusion Criteria:

Patients must have/be: metastatic pancreatic ductal adenocarcinoma (measurable or non-measurable); disease progression after prior gemcitabine or gemcitabine-containing therapy in a neoadjuvant, adjuvant (only if distant metastases occurred within 6 months of completing adjuvant therapy), locally advanced, or metastatic setting; KPS≧70; adequate hematologic (including absolute neutrophil count>1,500/μL), hepatic (including normal serum total bilirubin and albumin levels≧3.0 g/dL), and renal function; and ≧18 years of age.

Patient Characteristics

A total of 76 sites in 14 countries enrolled 417 patients between January 2012 and September 2013. Patient demographics and baseline clinical characteristics were well balanced across treatment arms (Table 8).

Treatment Exposure

The mean duration of treatment exposure was 18.5 weeks (median, 8.7 weeks; range, 2-115 weeks) in the nal-IRI+5-FU/LV arm, 12.3 weeks (median, 8.9 weeks; range, 3-69 weeks) in the nal-IRI arm, and 10.8 weeks (median, 6.0 weeks; range, 1-68 weeks) in the 5-FU/LV control arm.

The mean relative dose intensity of nal-IRI was 83% in the combination arm and 90% in the monotherapy arm.

TABLE 8
Patient Demographic and Baseline Clinical Characteristics

| | | 5-FU/LV | | 5-FU/LV |
|---|---|---|---|---|
| | nal-IRI + | combination | nal-IRI | monotherapy |
| | 5-FU/LV | control | monotherapy | control |
| Parameter | (n = 117) | (n = 119) | (n = 151) | (n = 149) |
| Median age | 63 (57-70) | 62 (55-69) | 65 (58-70) | 63 (55-69) |
| (IQR), y | | | | |
| KPS, % | | | | |
| 100 | 15 | 14 | 15 | 15 |
| 90 | 44 | 34 | 42 | 36 |
| 80 | 32 | 43 | 33 | 41 |
| 70 | 6 | 8 | 10 | 7 |
| 50-60 | 3 | 0 | 0 | 0 |
| Race, % | | | | |
| Caucasian | 62 | 64 | 59 | 62 |
| East Asian | 29 | 30 | 34 | 34 |
| Other | 9 | 6 | 7 | 5 |
| CA19-9 ≧ | 81 | 80 | 86 | 81 |
| 40 U/mL, %^{a} | | | | |
| Pancreatic head | 65 | 58 | 66 | 54 |
| tumor, % | | | | |
| Prior lines of | | | | |
| metastatic | | | | |
| therapy, % | | | | |
| 0^{b} | 13 | 13 | 11 | 13 |
| 1 | 53 | 56 | 57 | 58 |
| 2 | 34 | 31 | 32 | 30 |

naI-IRI, nanoliposomal irinotecan; 5-FU, 5-flurouracil; LV, leucovorin; IQR, interquartile range; KPS, Karnofsky performance status; CA 19-9, carbohydrate antigen 19-9.
^{a}Includes only patients who had a measured CA 19-9 value prior to treatment. Data were missing for 3 patients in the nal-IRI + 5-FU/LV group and 5 patients each in the ral-IRI monotherapy and 5-FU/LV groups.
^{b}Patients received neoadjuvant, sdjuvant, or locally advanced treatment, but had no previous therapy for metatstatic disease.

Efficacy

After 378 events, nal-IRI+5-FU/LV retained an OS advantage relative to 5-FU/LV (Table 9 and FIG. 16A).

With events in nearly all patients, the Kaplan-Meier OS curves converge at approximately 20 months, with 19 (16.2%) patients surviving beyond 20 months.

No OS advantage was observed with nal-IRI monotherapy versus 5-FU/LV (FIG. 16B).

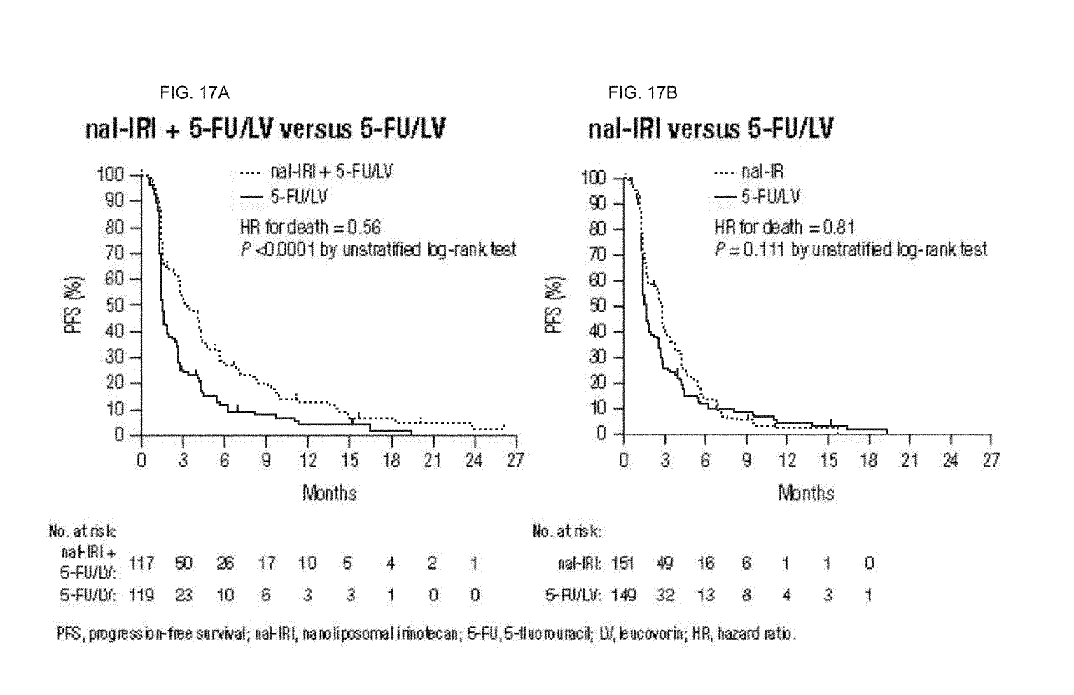

Median PFS was 3.1 months for nal-IRI+5-FU/LV versus 1.5 months for the 5-FU/LV combination control, and was 2.6 months for nal-IRI monotherapy compared with 1.6 months for the 5-FU/LV monotherapy control (Table 9 and FIG. 17).

ORR was higher than 5-FU/LV control for both nal-IRI+5-FU/LV (difference of 16% [95% CI, 9-24]) and nal-IRI monotherapy (difference of 5% [95% CI, 1-9]; Table 9)

TABLE 9
Summary of the Updated Efficacy

| | naI-IRI + | | | | | |
|---|---|---|---|---|---|---|
| | 5-FU/LV | 5-FU/LV | Treatment | nal-IRI | 5-FU/LV | Treatment |
| Endpoint | (n = 117) | (n = 119) | effect^{a} | (n = 151) | (n = 149) | effect^{a} |
| Median OS (95% CI), | 6.2 (4.8-8.4) | 4.2 (3.3-5.3) | HR 0.75 | 4.9 (4.2-5.6) | 4.2 (3.6-4.9) | HR 1.08 |
| months | | | P = 0.042 | | | P = 0.513 |
| OS rate at 6 months | 53 (44-62) | 38 (29-47) | — | | | — |
| (95% CI), % | | | | | | |
| OS rate at 12 months | 26 (18-35) | 16 (10-24) | — | | | — |
| (95% CI), % | | | | | | |
| Median PFS (95% CI), | 3.1 (2.7-4.2) | 1.5 (1.4-1.8) | HR 0.56 | 2.7 (2.1-2.9) | 1.6 (1.4-1.8) | HR 0.81 |
| months | | | P < 0.0001 | | | P = 0.111 |
| ORR (95% CI), %^{b} | 17 (10-24) | 1 (0-2) | P < 0.0001 | 6 (2-10) | 1 (0-2) | P = 0.020 |
| Best overall | | | | | | |
| response, % | | | | | | |
| Partial response^{b} | 17 | 1 | — | 6 | 1 | — |
| Stable disease^{c} | 33 | 22 | — | 36 | 24 | — |
| Progressive disease | 29 | 47 | — | 34 | 48 | — |
| Other^{d} | 3 | 2 | — | 2 | 1 | — |
| Not evaluable | 19 | 29 | — | 23 | 27 | — |

naI-IRI, nanoliposomal irinotecan;
5-FU, 5-flurouracil;
LV, leucovorin;
OS, overall survival;
CI, confidence interval;
HR, hazard ratio;
PFS, progression-free survival;
ORR, objective response rate.
^{a}Unstratified HR and log-rank P value.
^{b}Designation of response did not require confirmation and was based solely on the investigator's assessment using RECIST v1.1 criteria.
^{c}Minimum duration for stable disease from baseline is 6 weeks from the date of randomization.
^{d}Patients without measurable (target) disease at baseline may have a best overall-response of non-complete response/non-partial response.

Safety/Tolerability

The safety profiles of nal-IRI+5-FU/LV and nal-IRI monotherapy described in the current updated analysis did not change appreciably from that reported in the primary analysis.

The most frequently reported grade≧3 TEAEs in the nal-IRI—containing arms were neutropenia, diarrhea, vomiting, and fatigue (Table 10).

TEAEs led to dose delay, reduction, and/or discontinuation in 73% of patients in the nal-IRI+5-FU/LV arm, 56% of patients in the nal-IRI monotherapy arm, and 37% of patients in the 5-FU/LV arm.

The most common reasons for dose reduction in the nal-IRI+5-FU/LV and nal-IRI monotherapy arms were gastrointestinal events (12% and 17%, respectively) and neutropenia (18% and 10%).

The rate of treatment discontinuation due to a TEAE was 12% with nal-IRI+5-FU/LV, 14% with nal-IRI, and 8% with 5-FU/LV; neutropenia, diarrhea, and vomiting were the most common reasons for discontinuation in the nal-IRI—containing arms.

Grade≧3 febrile neutropenia occurred in 2 (2%) patients receiving nal-IRI+5-FU/LV and 6 (4%) patients receiving nal-IRI; 1 and 5 patients, respectively, required a dose reduction but no patient discontinued treatment due to febrile neutropenia.

No additional deaths due to treatment-related TEAEs have been reported since the primary analysis.

TABLE 10
Grade ≧3 TEAE (Treatment Emergent Adverse Event), %

| | nal-IRI + | nal-IRI | |
|---|---|---|---|
| | 5-FU/LV | monotherapy | 5-FU/LV |
| Grade ≧3 TEAE, % | (n = 117) | (n = 151) | (n = 134) |
| Any TEAE | 80 | 76 | 56 |
| Neutropenia^{a} | 28 | 15 | 2 |
| Fatigue | 14 | 6 | 4 |
| Diarrhea | 13 | 21 | 5 |
| Vomiting | 12 | 14 | 4 |
| Anemia | 9 | 11 | 7 |
| Asthenia | 8 | 7 | 7 |
| Nausea | 8 | 5 | 3 |
| While blood cell | 8 | 3 | 0 |
| count decreased | | | |
| Abdominal pain | 7 | 8 | 7 |
| Decreased appetite | 5 | 9 | 2 |
| Hypokalemia | 3 | 12 | 2 |
| Hyponatremia | 3 | 6 | 2 |
| Hyperglycemia | 2 | 5 | 2 |

TEAE, treatment-emergent adverse event; nal-IRI, nanoliposomal irinotecan; 5-FU, 5-fluoruracil; LV, leucovorin. The tabel includes all grade ≧3 TEAEs reproted for ≧5% of patients in any treatment arm.
^{a}Neutropenia includes agranulocytosis, febrile neutropenia, granulocytopenia, neutropenia, neutropenic sepsis, decreased neutrophil count, and pancytopenia.

In this analysis of updated data from the NAPOLI-1 trial, the previously described OS and PFS benefits were maintained for nal-IRI+5-FU/LV compared with 5-FU/LV alone. Convergence of the OS curves at 20 months (with 19 [16%] patients surviving beyond 20 months) is likely a reason for the observed attenuation of the OS HR estimate and unstratified log-rank P value. No new safety concerns were detected with nal-IRI as monotherapy or in combination with 5-FU/LV.

### Example 3: Alternative Analysis of the NAPOLI-1 Study: Effect of Baseline Carbohydrate Antigen 19-9 (CA 19-9) Level on Overall Survival in the NAPOLI-1 Phase 3 Sstudy

The objective of the current analysis of the NAPOLI-1 trial is to evaluate a potential predictive and/or prognostic effect between baseline CA 19-9 levels and efficacy in patients receiving nal-IRI+5-FU/LV versus 5-FU/LV alone.

CA 19-9 Assessment

Blood samples were taken at baseline and every 6 weeks thereafter until disease progression, initiation of a new antineoplastic treatment, or withdrawal of consent and assessed for CA 19-9 levels by a central lab in order to evaluate its predictive and prognostic roles.

CA 19-9 Quartile Analysis

The results of baseline CA 19-9 measurements were divided into quartiles (Q1-Q4). Treatment comparisons were carried out by an unstratified Cox proportional hazards regression model to estimate hazard ratios and corresponding 95% CI s for the effect of baseline CA 19-9 levels on OS, PFS, and ORR.

Quartile ranges were based on 404 available baseline CA 19-9 values from the 417 randomized patients in NAPOLI-1. Of the 236 patients randomized to receive nal-IRI+5-FU/LV or 5-FU/LV alone (after the protocol amendment), 218 received study treatment and had a baseline CA 19-9 measurement, and were included in this analysis (Table 11).

TABLE 11
CA 19-9 Quartiles

| | CA19-9 level | nal-IRI + | |
|---|---|---|---|
| | (U/mL) | 5-FU/LV, n | 5-FU/LV, n |
| Quartile 1 | <120 | 27 | 31 |
| Quartile 2 | 120 to <1,542 | 35 | 25 |
| Quartile 3 | 1,542 to <12,815 | 27 | 21 |
| Quartile 4 | ≧12,815 | 26 | 26 |

CA 19-9, carbohydrate antigen 19-9;
nal-IRI, nanoliposomal irinotecan;
5-FU, 5-flurouracil;
LV, leucovorin

Association of Baseline CA 19-9 Levels with Efficacy

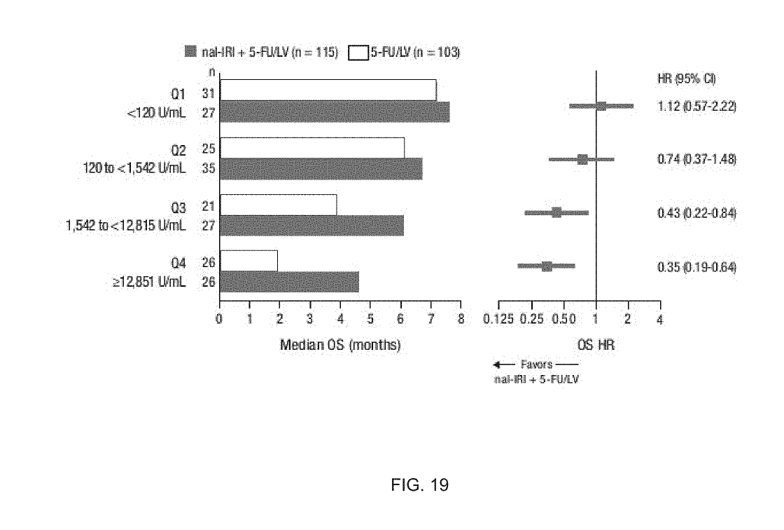

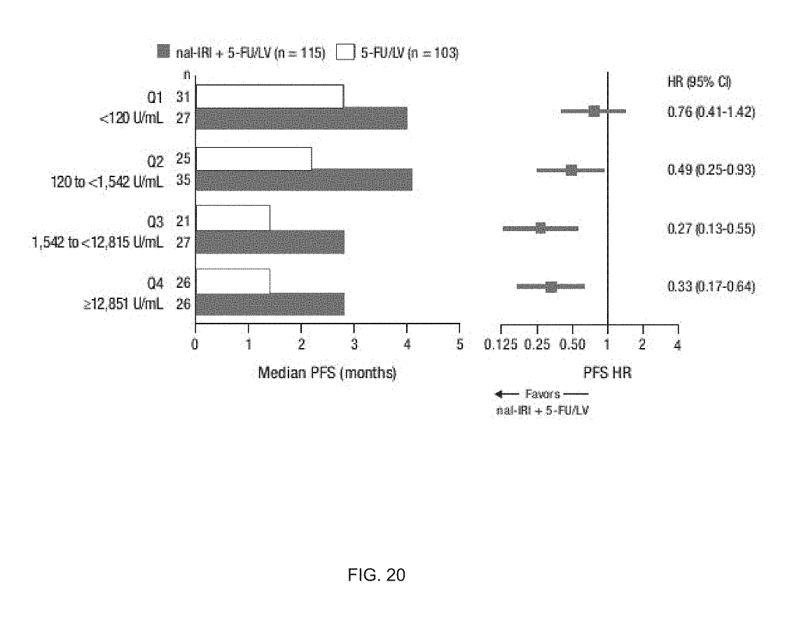

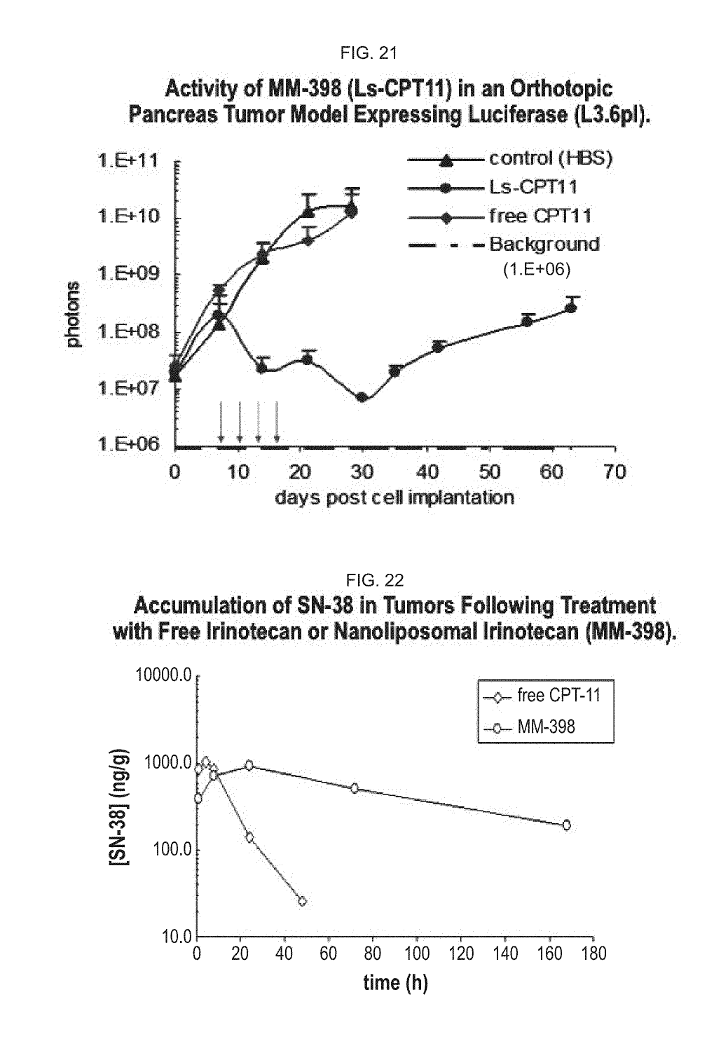

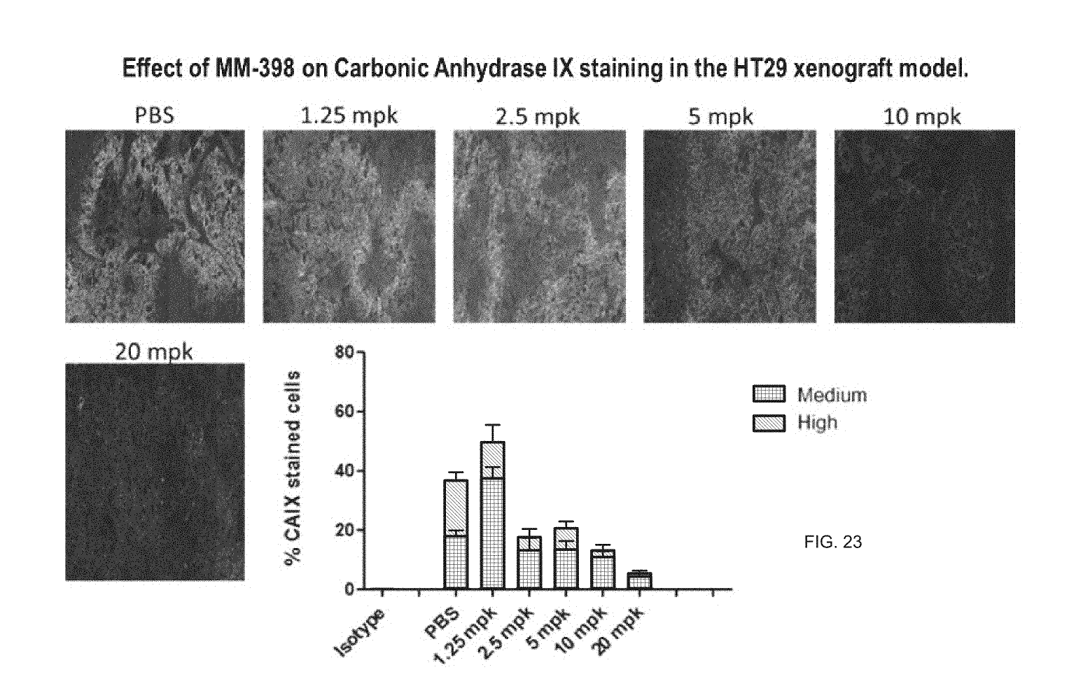

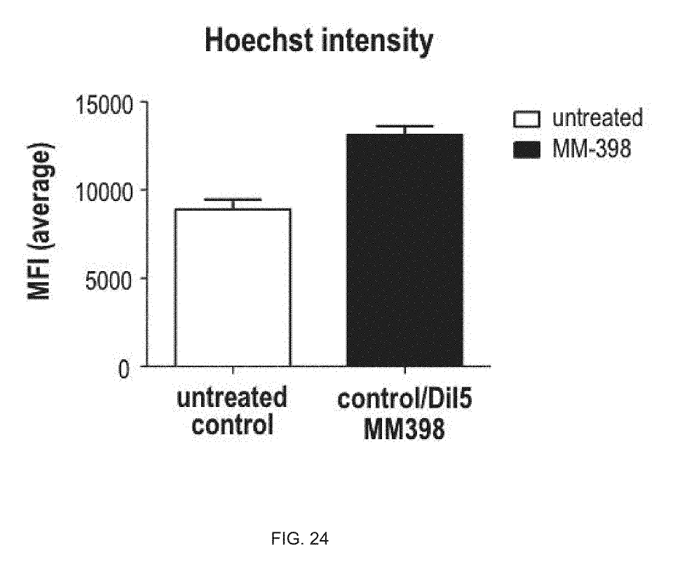

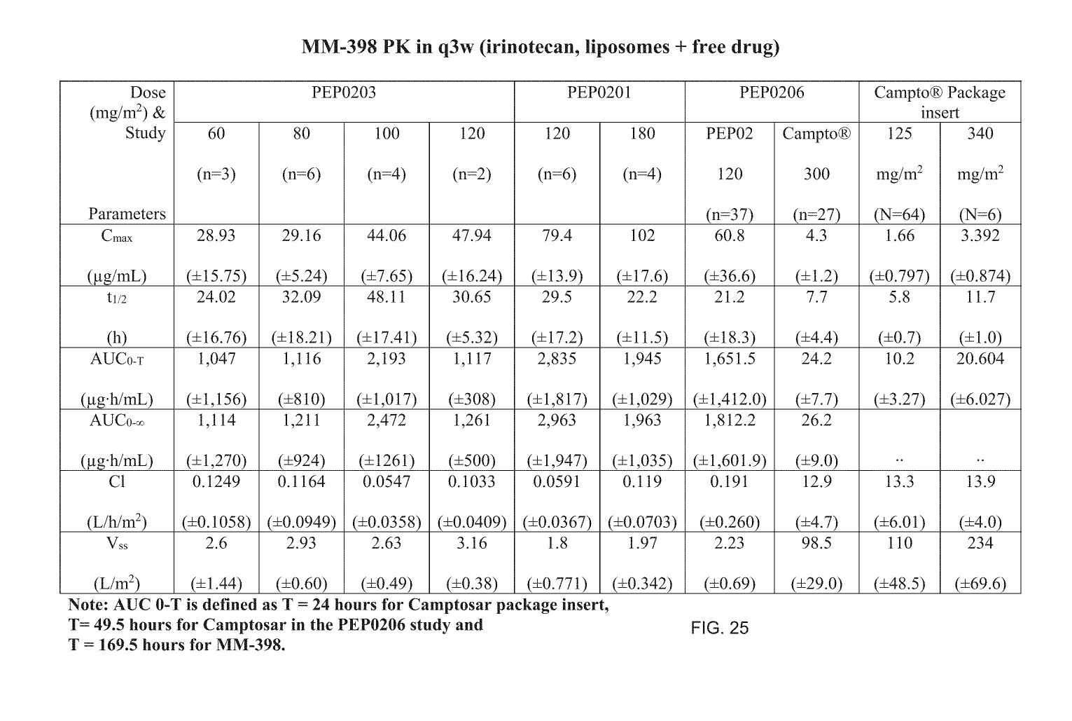

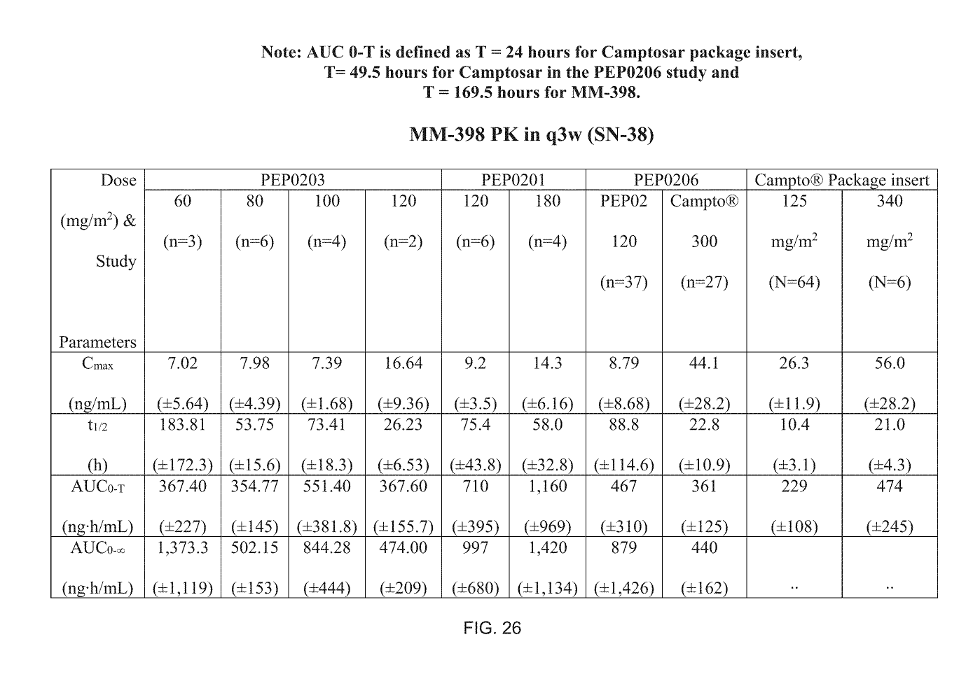

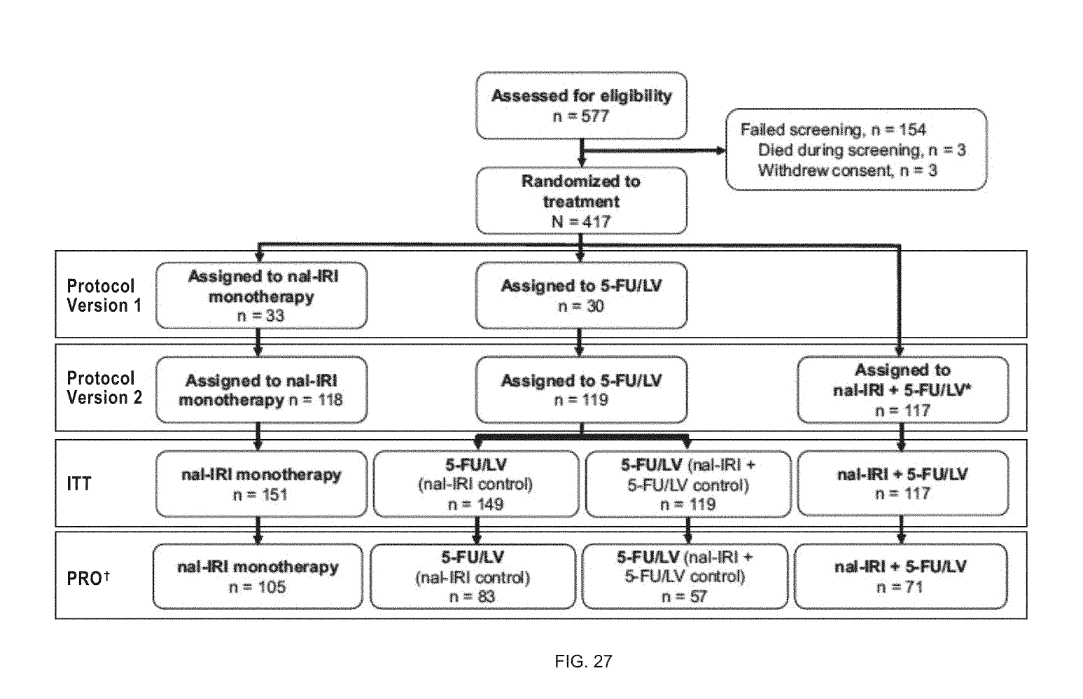

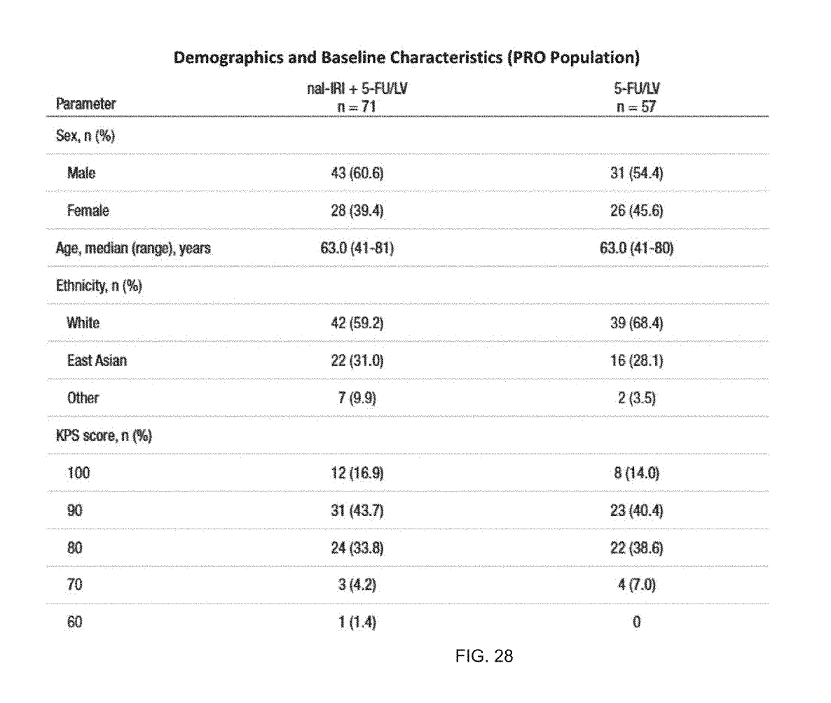

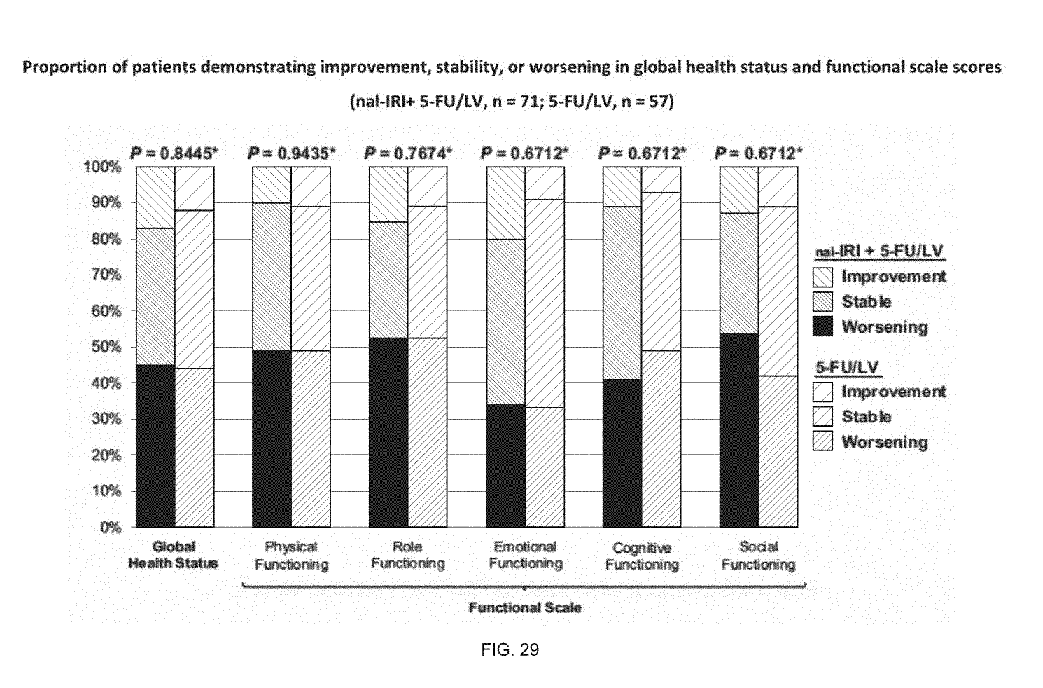

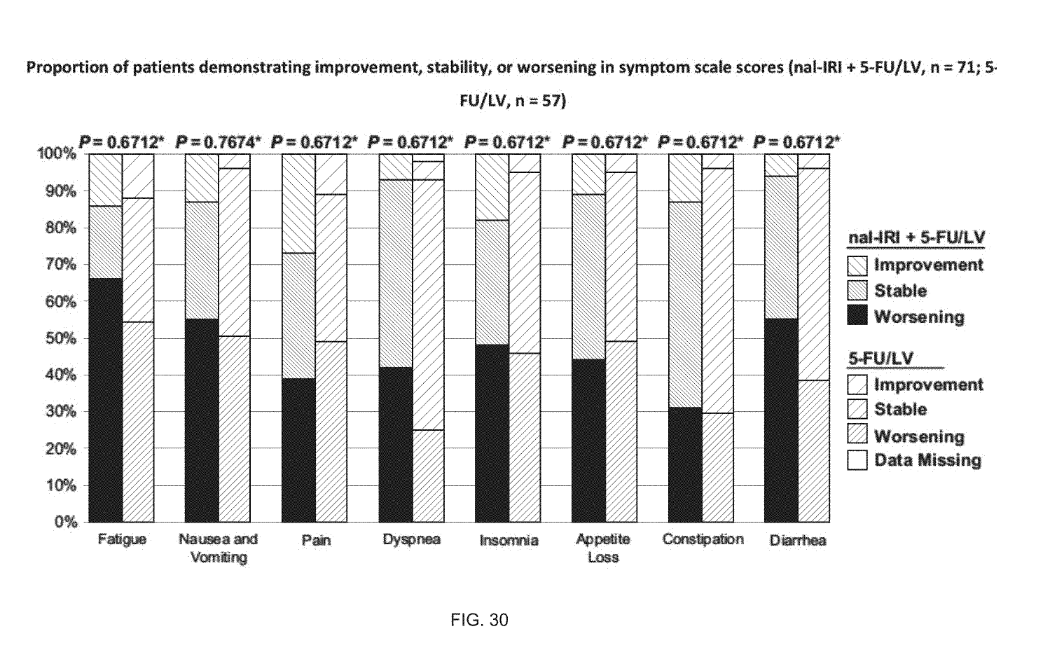

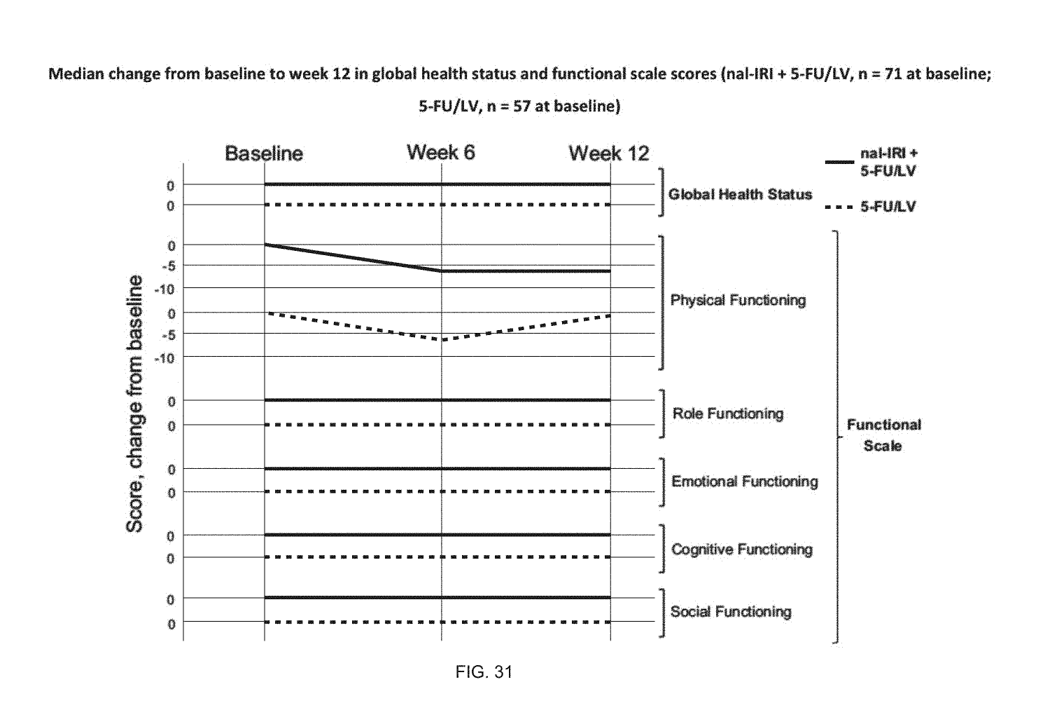

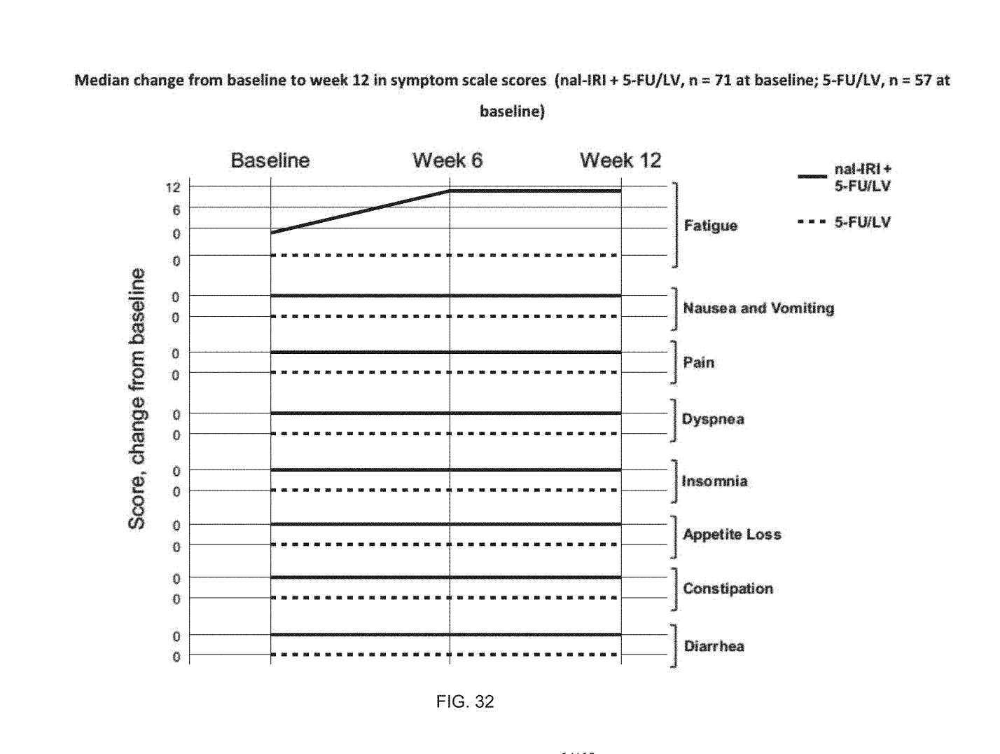

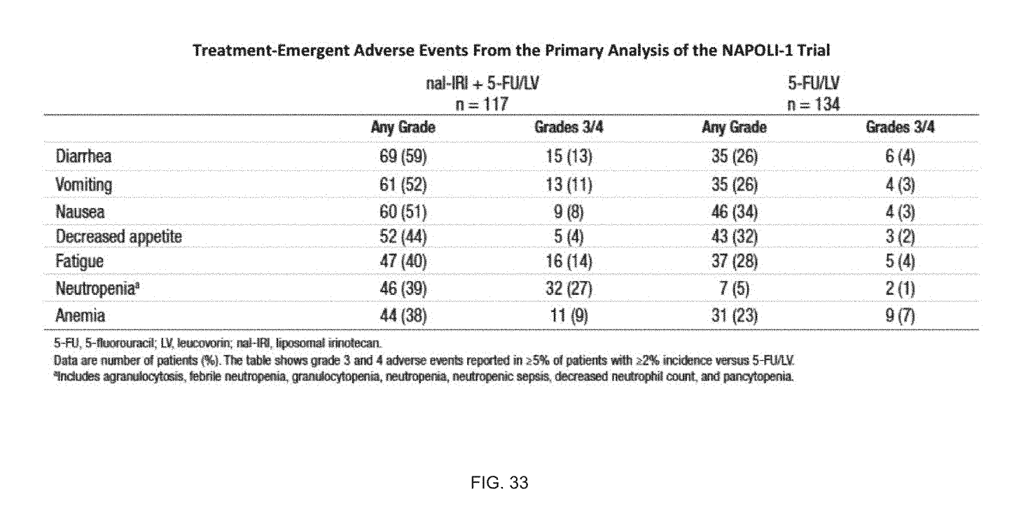

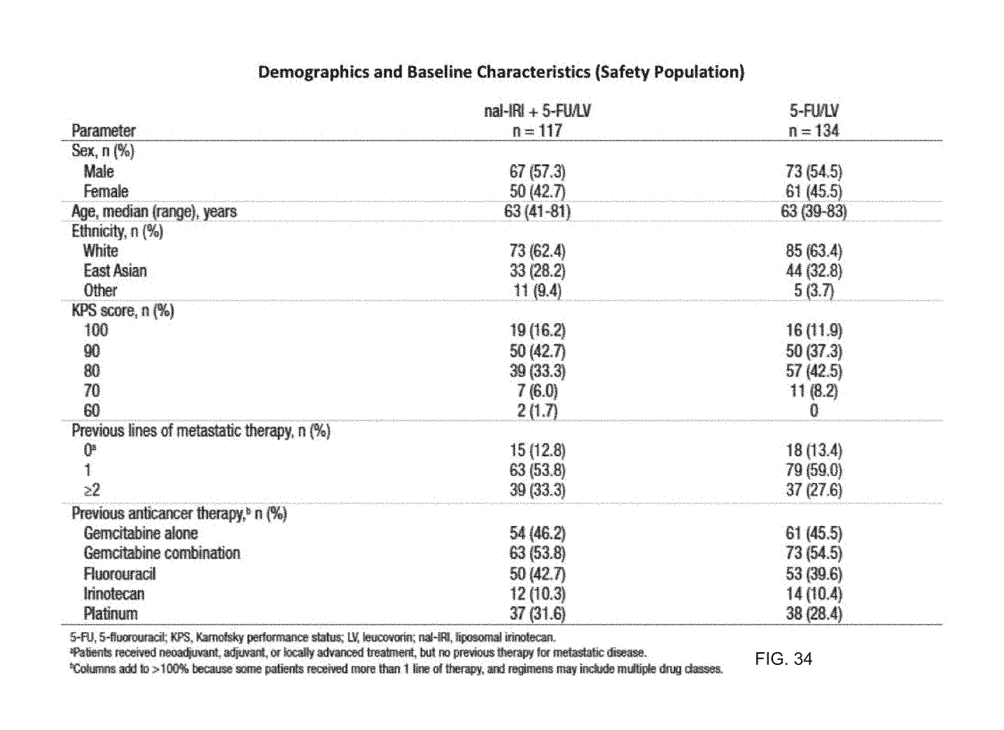

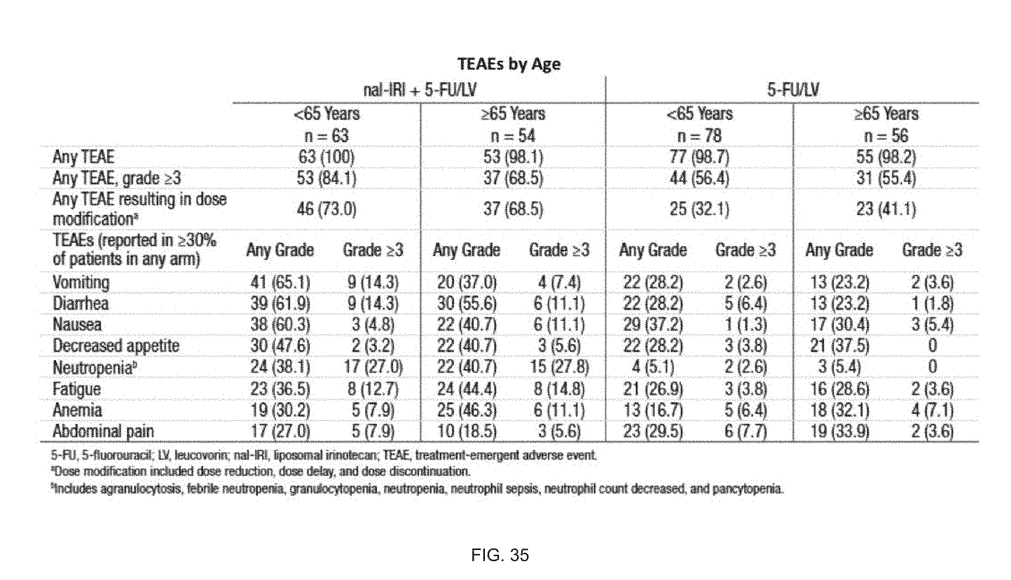

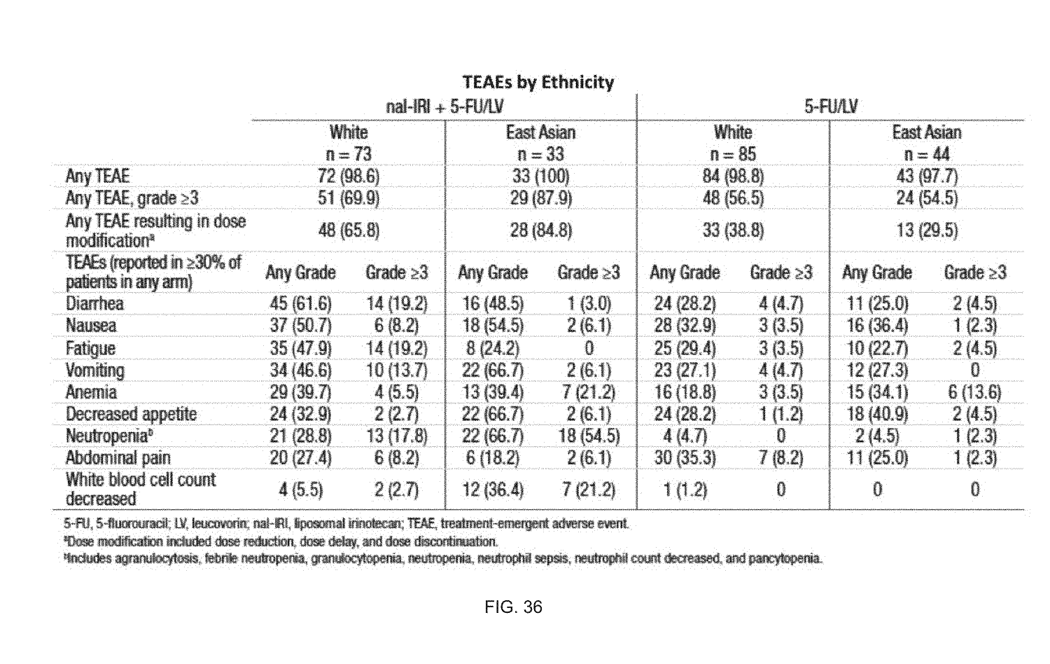

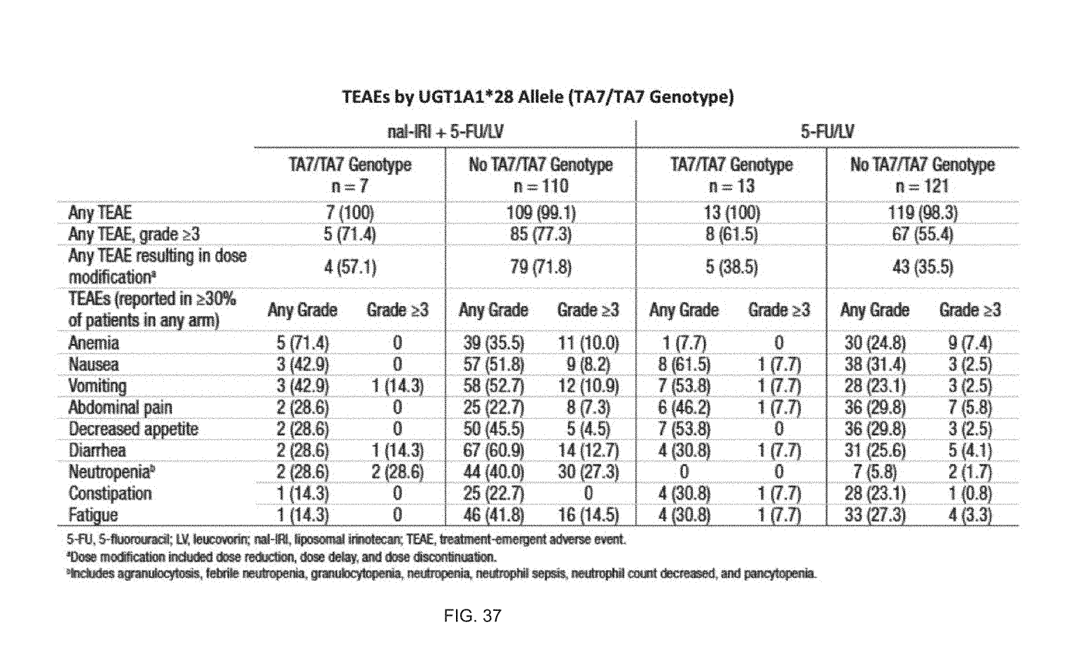

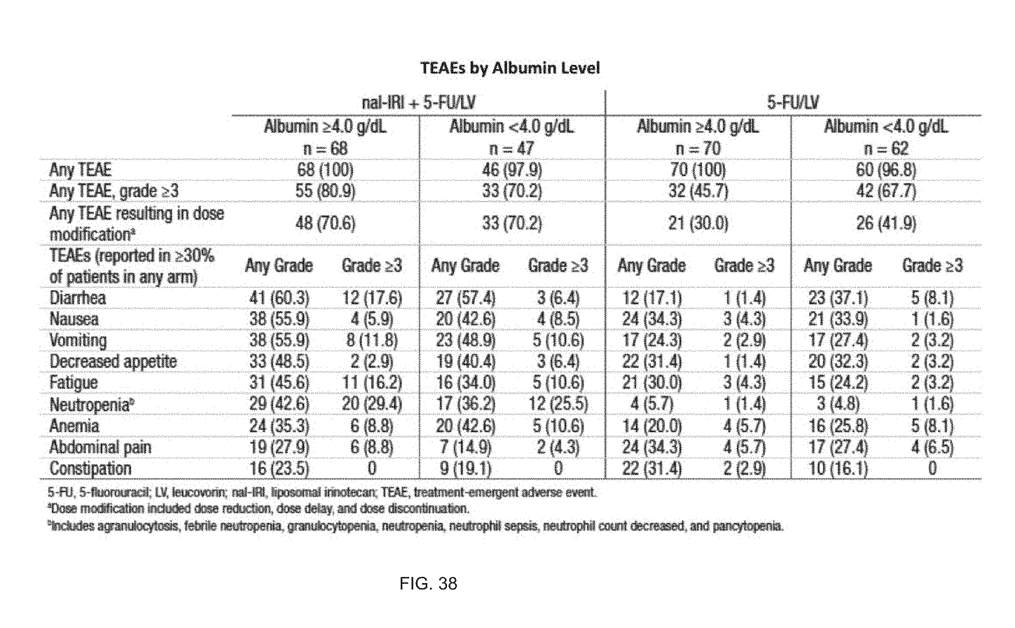

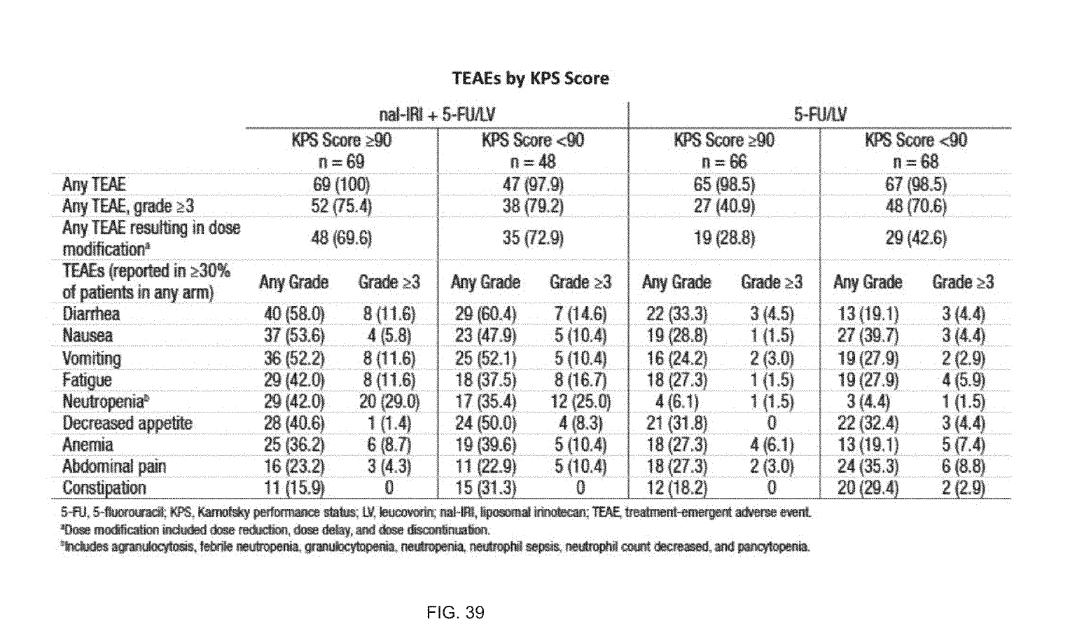

Results show a greater treatment effect on OS with higher CA 19-9 level relative to 5-FU/LV. CA 19-9 response (>50% decline from baseline) was superior with nal-IRI.5-FU/LV compared with 5-FU/LV (29% vs. 9%; P=0.0006). Nal-IRI alone did not show a statistical improvement in survival. A greater treatment effect of nal-IRI+5-FU/LV on OS and PFS relative to 5-FU/LV was observed with higher baseline CA 19-9 levels (U/mL) (FIG. 19 and FIG. 20) and table 12.

TABLE 12
Association Of Baseline CA 19-9 Levels with Overall Survival (OS)

| | Q2 | Q3 | |
|---|---|---|---|
| Q1 | 120 ≦ CA 19-9 < | 1,542 ≦ CA 19-9 < | Q4 |
| CA 19-9 < 120 | 1,542 | 12,815 | CA 19-9 > 12,815 |

Median OS, months (n in each group)

| | | | | | | | | |
|---|---|---|---|---|---|---|---|---|
| nal-IRI + | 7.6 | (n = 27) | 6.7 | (n = 35) | 6.1 | (n = 27) | 4.6 | (n = 26) |
| 5-FU/LV | | | | | | | | |
| (n = 117) | | | | | | | | |
| 5-FU/LV | 7.2 | (n = 31) | 6.1 | (n = 25) | 3.8 | (n = 21) | 1.9 | (n = 26) |
| (n = 103) | | | | | | | | |
| HR for death | 1.12 | (0.57, 2.22) | 0.74 | (0.37, 1.48) | 0.43 | (0.22, 0.84) | 0.35 | (0.19, 0.64) |
| (95% Cl) | | | | | | | | |

Objective Response Rate (ORR) was higher in the nal-IRI+5-FU/LV arm compared with the 5-FU/LV arm across all quartiles of baseline CA 19-9 levels (Table 13)

TABLE 13
Association Of Baseline CA 19-9 Levels with Objective Response Rate (ORR).

| | CA19-9 (U/mL) quartile | | | | | | | |
|---|---|---|---|---|---|---|---|---|
| | | | Q2 | | Q3 | | | |
| | Q1 | | 120 ≦ CA19-9 < | | 1,542 ≦ CA19-9 < | | Q4 | |
| ORR, n/N (%) | CA19-9 < 120 | | 1,542 | | 12,815 | | CA19-9 ≧ 12,815 | |
| nal-IRI + | 2/27 | (7) | 7/35 | (20) | 7/27 | (26) | 3/26 | (12) |
| 5-FU/LV | | | | | | | | |
| (n = 115) | | | | | | | | |
| 5-FU/LV | 0/31 | (0) | 1/25 | (4) | 0/21 | (0) | 0/26 | (0) |
| (n = 103) | | | | | | | | |

CA 19-9, carbohydrate antigen 19-9;
nal-IRI, nanoliposomal irinotecan;
5-FU, 5-flurouracil;
LV, leucovorin

nal-IRI+5-FU/LV significantly improved OS and PFS compared with 5-FU/LV control in the NAPOLI-1 trial of patients with mPAC following gemcitabine-based therapy. The results show a greater treatment effect on OS with higher CA 19-19 levels relative to 5-FU. The observed OS and PFS benefits for nal-IRI+5-FU/LV over 5-FU/LV were greatest among patients with the highest baseline CA 19-9 levels. ORR was greater with nal-IRI+5-FU/LV relative to 5-FU/LV control in the overall population, and there was no clear trend in impact on ORR relative to baseline CA 19-9. These results suggest that baseline CA 19-9 levels are associated with the treatment effect observed for the combination of nal-IRI+5-FU/LV in mPAC patients and that CA 19-9 serum level can provide important information with regards to overall survival.

### Example 4: Activity of MM-398 in an Orthotopic Pancreas Tumor Model Expressing Luciferase (L3.6pl)

The anti-tumor activity of MM-398 was assessed in an orthotopic pancreatic cancer model (L3.6pl), a highly hypoxic preclinical tumor model. Approximately 2.5×10⁻⁵ L3.6pl pancreatic tumor cells were implanted by direct injection into the pancreas. The bioluminescence images (BLI) were followed over time for tumor burden detection/quantitation. MM-398 and free irinotecan were dosed at a dose of 20 mg/kg/dose weekly for three weeks. As shown in FIG. 1, MM-398 (liposomal CPT11) had significant anti-tumor activity, as compared to a control (FIBS) and free CPT11.

### Example 5: Accumulation of SN-38 in Tumors Following Treatment with Free Irinotecan or Liposomal Irinotecan (MM-398)

It was hypothesized that the anti-tumor activity observed in the orthotopic pancreatic cancer model is due to the effect of macrophages in converting irinotecan to the more active SN-38 locally. To test this hypothesis, human colon cancer cells (HT-29) were injected subcutaneously into SCID mice, 40 mg/kg of free irinotecan or MM-398 was injected intravenously when the tumors reached 1000 mm³ in size. Tumor-bearing mice were sacrificed at different time points, tumors from both groups were extracted and the concentrations of SN-38 were measured.

As shown in FIG. 2, there was a 20-fold increase in the tumor AUC_{SN-38} for MM-398 as compared to free irinotecan. The long duration of exposure allows for prolonged exposure of the slow proliferating cancer cells to the active metabolite as they progress through the cell cycle. In addition, this activity was also hypothesized to result from a reduction in intra-tumoral hypoxia, and the subsequent downstream effects on angiogenesis, metastasis, and the immunosuppressive environment in tumors.

### Example 6: Effect of MM-398 on Carbonic Anhydrase IX Staining in a HT29 Xenograft Model

To test whether MM-398 reduces markers of hypoxia, experiments were conducted in a human colon cancer cell (HT-29) model. Specifically, HT-29 cells were injected subcutaneously into nude mice, on day 13 either PBS control or 1.25, 2.5, 5, 10 or 20 mg/kg MM-398 was injected intravenously. MM-398 was dosed once a week for 4 weeks at the indicated doses. Tumors from both groups (n=5) were extracted 24 hours after the last dose. Frozen tumor sections were used for immunohistochemical staining of Carbonic Anhydrase IX (CAIX). Quantification of CAIX staining was performed using Definiens® (Definiens AG, Munich) software.

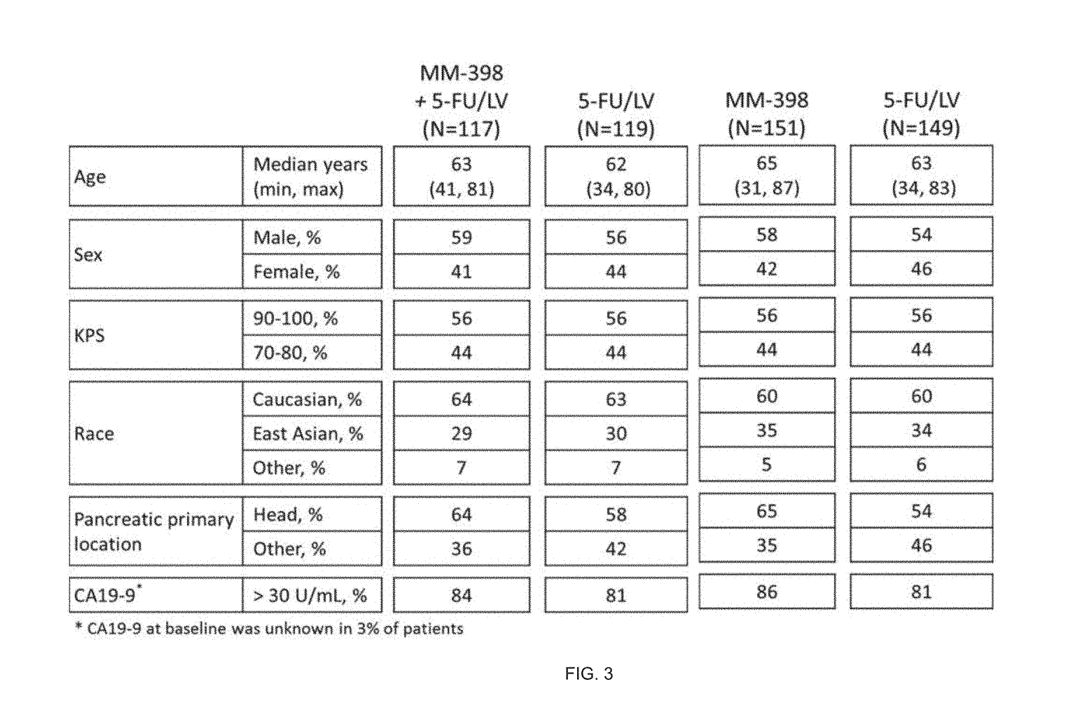

As shown in FIG. 3, MM-398 reduced markers of hypoxia. Specifically, the graphs in FIG. 3 show the percentage of cells that stained with medium (middle third) or high (top third) intensity for CAIX. Representative samples from each group are shown as well as the group average (mean+/−stdev). MM-398 treatment modifies the tumor microenvironment by decreasing the percentage of both medium and high CAIX positive cells in a dose-dependent manner. As hypoxia is a hallmark of resistant and aggressive disease, a reduction in hypoxia is expected to make tumor cells more sensitive to chemotherapies.

### Example 7: MM-398 Increases Perfusion of Hoechst Stain

In addition to changing the chemosensitivity of tumor cells through modification of the tumor microenvironment, lowering hypoxia can indicate improved tumor vascularization, which can facilitate delivery of small molecule therapies. MM-398 treatment led to increased microvessel density 6 days after treatment as measured by CD31 (platelet endothelial cell adhesion molecule) staining in an HT29 xenograft study. To further assess the effect of MM-398 on small molecule tumor vascularization, a Hoechst 33342 perfusion experiment was conducted. Specifically, a primary pancreatic tumor was grown in NOD-SCID mice and given one dose of MM-398 (20 mg/kg). After 24 hours, Hoechst 33342 stain was administered 20 minutes prior to sacrificing the animal. As shown in FIG. 4, the increase in stain intensity in treated mice was statistically significant, p<0.001. These data indicate that MM-398 modifies the tumor microenvironment in a manner that should make tumors more susceptible to agents such as 5-FU/LV, through decreasing tumor hypoxia and increasing small molecule perfusion.

### Example 8: MM-398 Pharmacokinetics in Humans (Phase I)

The pharmacokinetic profile of MM-398 single agent was investigated in a phase I clinical study (PEP0201) in patients at 60, 120 or 180 mg/m² dose levels and in a phase II clinical trial in gastric cancer patients (PEP0206) at 120 mg/m². Plasma levels of total irinotecan, SN-38 and encapsulated irinotecan were measured in these studies.

The peak serum concentrations of total irinotecan (Cₘₐₓ) ranged from 48-79 μg/ml for 120 mg/m² of MM-398, which was approximately 50 fold higher than 125 mg/m² free irinotecan. The total irinotecan half-life (t_{1/2}) for MM-398 ranged from 21 to 48 hours, which was approximately 2-3 fold higher than 125 mg/m² of free irinotecan. Overall, total irinotecan exposure at one week (AUC 0-T) ranged from 1200-3000 (μg*h/ml) at a dose of 120 mg/m² of MM-398, approximately 50-100 fold higher than 300 mg/m² of free irinotecan. In contrast, SN38 Cₘₐₓ levels at 120 mg/m² of MM-398 ranged from 9 to 17 ng/ml, which was approximately 50% less than free irinotecan at 125 mg/m². Overall, exposure of SN38 at one week (AUC 0-T) ranged from 474 to 997 ng*/ml and was only 1-2 fold higher than achieved by free irinotecan at 300 mg/m². For both SN38 and total irinotecan, AUC increased less than proportionally with dose of MM-398. The PK parameters of encapsulated irinotecan almost matched that of total irinotecan indicates that most of irinotecan remained encapsulated in the liposomes during circulation. The MM-398 PK parameters were not significantly changed when combined with 5-FU/LV. FIGS. 5 and 6 summarize the PK findings in previous studies of MM 398.

### Example 9: Phase 1 Dose Escalation Study

A regimen combining fluorouracil, leucovorin, and MM-398 was studied in a phase 1 trial of solid tumors in 16 subjects, of whom 5 were patients with pancreatic cancer. The objective tumor response rate, duration of response, and disease control rate were efficacy endpoints of the study. Among the 15 efficacy-evaluable patients, 2 (13.3%) had confirmed PR, 9 (60.0%) had SD, and 4 (26.7%) had PD. The overall disease control rate was 73.3%. Partial response was observed in one gastric cancer patient (at 80 mg/m² dose level) and one breast cancer patient (at 100 mg/m² dose level), with the duration of response of 142 and 76 days, respectively. Among the 6 patients who received the MTD dose of 80 mg/m², there were 1 PR, 4 SD and 1 PD. The tumor response rate and disease control rate were 16.7% and 83.3%, respectively. The main DLTs were grade 3 diarrhea, leucopenia, neutropenia and febrile neutropenia. The MTD for MM-398 was 80 mg/m².

In the phase 1 dose-escalation study of MM-398 in combination with 5-FU/LV in advanced solid tumors (PEP0203), a total of 401 episodes of AE were reported from the 16 treated subjects (safety population), of which 74 (18.4%) were of CTC grade 3 or above. Among all AEs, 231 (57.6%) were considered by the investigators to be treatment-related. The most common treatment-related AEs, included nausea (81.3%), diarrhea (75.0%), vomiting (68.8%), fatigue (43.8%), mucositis (43.8%), leucopenia (37.5%), neutropenia (37.5%), weight loss (37.5%), anemia (31.3%), and alopecia (31.3%). Acute cholinergic diarrhea was rarely observed. Table 14 provides the incidence of treatment-emergent adverse events by maximum CTC grade and by causality (incidence 20%), as seen in the PEP0203 study. Table 15 provides the incidence of grade 3 or higher treatment-emergent adverse events seen in the 5 pancreatic cancer patients treated in the PEP0203 study.

TABLE 14
Incidence of treatment-emergent adverse events by maximum CTC grade
and by causality (incidence ≧20%) in the PEP0203 Study

| | | Severity | | | | | |
|---|---|---|---|---|---|---|---|
| System organ class | Total | (Grade)¹ | | | | Causality² | |
| Preferred Term | (N = 16) | I | II | III | IV | es | o |
| Blood and lymphatic | | | | | | | |
| system disorders | | | | | | | |
| Anemia | 7 (43.8%) | 3 | 2 | 2 | 0 | 5 | |
| Leucopenia | 6 (37.5%) | 0 | 3 | 2 | 1 | 6 | |
| Neutropenia | 6 (37.5%) | 0 | 2 | 3 | 1 | 6 | |
| Gastrointestinal disorders | | | | | | | |
| Abdominal pain | 7 (43.8%) | 3 | 2 | 2 | 0 | 3 | |
| Constipation | 6 (37.5%) | 3 | 3 | 0 | 0 | 0 | |
| Diarrhea | 12 (75.0%) | 3 | 4 | 5 | 0 | 12 | |
| Nausea | 13 (81.3%) | 6 | 6 | 1 | 0 | 13 | |
| Vomiting | 12 (75.0%) | 3 | 8 | 1 | 0 | 11 | |
| General disorders and | | | | | | | |
| administration site | | | | | | | |
| conditions | | | | | | | |
| Fatigue | 8 (50.0%) | 4 | 3 | 1 | 0 | 7 | |
| Mucosal inflammation | 7 (43.8%) | 4 | 3 | 0 | 0 | 7 | |
| Pyrexia | 7 (43.8%) | 3 | 4 | 0 | 0 | 2 | |
| Infections and infestations | | | | | | | |
| Infection | 6 (37.5%) | 0 | 3 | 3 | 0 | 2 | |
| Investigations | | | | | | | |
| ALT increased | 5 (31.3%) | 3 | 2 | 0 | 0 | 4 | |
| AST increased | 4 (25.0%) | 3 | 1 | 0 | 0 | 1 | |
| Weight decreased | 8 (50.0%) | 4 | 4 | 0 | 0 | 6 | |
| Metabolism and | | | | | | | |
| nutrition disorders | | | | | | | |
| Anorexia | 4 (25.0%) | 1 | 2 | 1 | 0 | 3 | |
| Hypoalbuminaemia | 4 (25.0%) | 0 | 3 | 1 | 0 | 0 | |
| Hypocalcaemia | 5 (31.3%) | 1 | 4 | 0 | 0 | 0 | |
| Hypokalaemia | 8 (50.0%) | 2 | 0 | 5 | 1 | 2 | |
| Hyponatraemia | 4 (25.0%) | 2 | 0 | 0 | 2 | 0 | |
| Nervous system disorders | | | | | | | |
| Dizziness | 4 (25.0%) | 4 | 0 | 0 | 0 | 1 | |
| Psychiatric disorders | | | | | | | |
| Insomnia | 4 (25.0%) | 4 | 0 | 0 | 0 | 1 | |
| Respiratory, thoracic and | | | | | | | |
| mediastinal disorders | | | | | | | |
| Cough | 5 (31.3%) | 3 | 1 | 1 | 0 | 0 | |
| Skin and subcutaneous | | | | | | | |
| tissue disorders | | | | | | | |
| Alopecia | 5 (31.3%) | 5 | 0 | 0 | 0 | 5 | |

¹Severity grading used the highest grading ever rated for each subject if the subject had such adverse event reported
²Defined as subject ever experienced AE related to the study drug in causality or not

TABLE 15
Incidence of Grade 3 or higher treatment-emergent adverse
events in pancreatic cancer patients in the PEP0203 Study

| Primary system organ | Overall | 60 mg/m2 | 80 mg/m2 | 120 mg/m2 |
|---|---|---|---|---|
| class | N = 5 | N = 1 | N = 3 | N = 1 |
| Preferred term | n (%) | n (%) | n (%) | n (%) |
| Any primary system | | | | |
| organ class | | | | |
| Total | 3 (60.0) | 0 | 2 (66.7) | 1 (100.0) |
| Infections and | | | | |
| infestations | | | | |
| Total | 3 (60.0) | 0 | 2 (66.7) | 1 (100.0) |
| Hepatitis viral | 1 (20.0) | 0 | 1 (33.3) | 0 |
| Infection | 1 (20.0) | 0 | 0 | 1 (100.0) |
| Pneumonia | 1 (20.0) | 0 | 1 (33.3) | 0 |
| Septic shock | 1 (20.0) | 0 | 1 (33.3) | 0 |
| Blood and lymphatic | | | | |
| system disorders | | | | |
| Total | 2 (40.0) | 0 | 1 (33.3) | 1 (100.0) |
| Lymphopenia | 1 (20.0) | 0 | 0 | 1 (100.0) |
| Neutropenia | 1 (20.0) | 0 | 1 (33.3) | 0 |
| White blood cell | 1 (20.0) | 0 | 0 | 1 (100.0) |
| disorder | | | | |
| Gastrointestinal | | | | |
| disorders | | | | |
| Total | 2 (40.0) | 0 | 1 (33.3) | 1 (100.0) |
| Diarrhea | 2 (40.0) | 0 | 1 (33.3) | 1 (100.0) |
| Abdominal pain | 1 (20.0) | 0 | 0 | 1 (100.0) |
| Gastrointestinal | 1 (20.0) | 0 | 1 (33.3) | 0 |
| haemorrhage | | | | |
| Investigations | | | | |
| Total | 2 (40.0) | 0 | 1 (33.3) | 1 (100.0) |
| Blood bilirubin | 1 (20.0) | 0 | 1 (33.3) | 0 |
| increased | | | | |
| Lipase increased | 1 (20.0) | 0 | 0 | 1 (100.0) |
| Neutrophil count | 1 (20.0) | 0 | 0 | 1 (100.0) |
| decreased | | | | |
| White blood cell | 1 (20.0) | 0 | 0 | 1 (100.0) |
| count decreased | | | | |
| Metabolism and | | | | |
| nutrition disorders | | | | |
| Total | 2 (40.0) | 0 | 1 (33.3) | 1 (100.0) |
| Hypoalbuminaemia | 1 (20.0) | 0 | 1 (33.3) | 0 |
| Hypokalaemia | 1 (20.0) | 0 | 1 (33.3) | 0 |
| Hyponatraemia | 1 (20.0) | 0 | 0 | 1 (100.0) |
| Hypophosphataemia | 1 (20.0) | 0 | 0 | 1 (100.0) |
| Respiratory, thoracic | | | | |
| and mediastinal | | | | |
| disorders | | | | |
| Total | 2 (40.0) | 0 | 1 (33.3) | 1 (100.0) |
| Dyspnoea | 1 (20.0) | 0 | 0 | 1 (100.0) |
| Pleural effusion | 1 (20.0) | 0 | 1 (33.3) | 0 |
| General disorders and | | | | |
| administration site | | | | |
| conditions | | | | |
| Total | 1 (20.0) | 0 | 0 | 1 (100.0) |
| Death | 1 (20.0) | 0 | 0 | 1 (100.0) |

### Example 10: Time Course of Select Treatment Emergent Adverse Events (TEAEs) in NAPOLI-1: A Phase 3 Study of Nal-IRI (MM-398)±5-Fluorouracil and Leucovorin (5-FU/LV) Vs 5-FU/LV in Metastatic Pancreatic Cancer (mPAC) Previously Treated with Gemcitabine-Based Therapy

Background: Liposomal irinotecan (nal-IRI, ONIVYDE®, MM-398) plus 5-FU/LV is approved in the US for patients (pts) with mPAC previously treated with gemcitabine-based therapy. Primary analysis from NAPOLI-1 (NCT01494506) showed a significant median survival advantage for nal-IRI+5-FU/LV vs 5-FU/LV (6.1 vs 4.2 mo; HR 0.67; 95% CI 0.49-0.92; P=0.012); Wang-Gillam et al., Lancet. 2016). The most common TEAEs included diarrhea, vomiting, nausea, decreased appetite, fatigue, neutropenia, and anemia. Here we report incidence and prevalence of selected TEAEs over time in NAPOLI-1.

Methods: Pts were randomly assigned to nal-IRI+5-FU/LV, nal-IRI, or 5-FU/LV. In this post-hoc analysis (data cutoff, Feb. 14, 2014), incidence (ie, first occurrence) and prevalence (ie, first occurrence, ongoing event, or recurrence) of selected TEAEs were analyzed by treatment period (first 6 wk [period 1], second 6 wk [period 2], and beyond second 6 wk [period 3]). Denominators for percentages were the number of pts in the risk set during each period (for incidence: pts still on treatment without a previous event; for prevalence: all safety-evaluable pts).

Results: 398 pts were treated with nal-IRI+5-FU/LV (n=117), nal-IRI (n=147), or 5-FU/LV (n=134). In the nal-IRI+5-FU/LV arm, most first occurrences of neutropenia, diarrhea, nausea, and vomiting were during the first 6 wk of treatment, with incidence and severity generally decreasing thereafter (Table 16). Similarly, prevalence and severity were highest in the first 6 wk and tended to decrease over time. Similar trends were observed in the nal-IRI and 5-FU/LV arms.

Conclusions: Neutropenia, diarrhea, nausea, and vomiting typically first occur early during the course of treatment with nal-IRI+5-FU/LV and tend to decrease in incidence and severity thereafter.

TABLE 16

| | nal-IRI + 5-FU/LV | | | nal-IRI | | | 5-FU/LV | | |
|---|---|---|---|---|---|---|---|---|---|
| | Period | | | Period | | | Period | | |
| Incidence, % | 1 | 2 | 3 | 1 | 2 | 3 | 1 | 2 | 3 |
| Neutropenia grade | n = 117 | n = 73 | n = 34 | n = 147 | n = 95 | n = 43 | n = 134 | n = 111 | n = 43 |
| 1 | 1 | 3 | 3 | 1 | 2 | 0 | 1 | 0 | 0 |
| 2 | 8 | 3 | 3 | 8 | 3 | 0 | 1 | 2 | 2 |
| 3 | 14 | 4 | 9 | 5 | 1 | 0 | 2 | 0 | 0 |
| 4 | 7 | 0 | 0 | 6 | 2 | 0 | 0 | 0 | 0 |
| 5 | 0 | 0 | 0 | 0 | 0 | 0 | 0 | 0 | 0 |
| Diarrhea grade | n = 117 | n = 51 | n = 24 | n = 147 | n = 46 | n = 11 | n = 134 | n = 89 | n = 32 |
| 1 | 21 | 4 | 0 | 25 | 11 | 18 | 15 | 5 | 6 |
| 2 | 17 | 12 | 4 | 20 | 7 | 9 | 2 | 1 | 0 |
| 3 | 12 | 0 | 4 | 16 | 4 | 0 | 2 | 1 | 3 |
| 4 | 0 | 0 | 0 | 1 | 0 | 0 | 0 | 0 | 0 |
| 5 | 0 | 0 | 0 | 1 | 0 | 0 | 0 | 0 | 0 |
| Nausea grade | n = 117 | n = 56 | n = 28 | n = 147 | n = 53 | n = 25 | n = 134 | n = 87 | n = 26 |
| 1 | 21 | 5 | 7 | 23 | 4 | 8 | 19 | 4 | 12 |
| 2 | 16 | 0 | 7 | 27 | 4 | 8 | 5 | 4 | 4 |
| 3 | 7 | 2 | 0 | 5 | 2 | 0 | 2 | 2 | 0 |
| 4 | 0 | 0 | 0 | 0 | 0 | 0 | 0 | 0 | 0 |
| 5 | 0 | 0 | 0 | 0 | 0 | 0 | 0 | 0 | 0 |
| Vomiting grade | n = 117 | n = 61 | n = 35 | n = 147 | n = 61 | n = 28 | n = 134 | n = 89 | n = 29 |
| 1 | 19 | 5 | 11 | 27 | 2 | 7 | 16 | 2 | 4 |
| 2 | 14 | 0 | 9 | 10 | 3 | 4 | 5 | 1 | 4 |
| 3 | 10 | 0 | 3 | 12 | 2 | 0 | 1 | 1 | 4 |
| 4 | 0 | 0 | 0 | 0 | 0 | 0 | 0 | 0 | 0 |
| 5 | 0 | 0 | 0 | 0 | 0 | 0 | 0 | 0 | 0 |

### Example 11: Quality-Adjusted Time without Symptoms or Toxicity (Q-TWiST) of Nanoliposomal Irinotecan (Nal-IRI; MM-398) Plus 5-Fluorouracil and Leucavorin (5-FU/LV) Vs 5-FU/LV Alone in Patients (Pts) with Metastatic Pancreatic Adenocarcinoma (mPAC) Previously Treated with Gemcitabine-Based Therapy

In the primary analysis of the Phase 3 NAPOLI-1 trial, nal-IRI+5-FU/LV significantly improved median overall survival (OS; 6.1 vs 4.2 months; hazard ratio [HR]=0.67; P=0.012) and progression-free survival (PFS; 3.1 vs 1.5 months; HR=0.56; P=0.0001) vs 5-FU/LV alone in mPAC patients (pts) previously treated with gemcitabine-based therapy. Here we report between-treatment differences in quality-adjusted survival using Q-TWiST methodology.

The total survival in NAPOLI-1 intent-to-treat cohort over 12 months was partitioned into time with adverse events grade≧3 toxicity (TWiST). Mean Q-TWiST was calculated by multiplying time spent in each health state by its respective utility. In the base case, the utility for toxicity (uTOX), relapse (uREL) and TWiST (uTWiST) were set to 0.5, 0.5 and 1.0. Non-parametric bootstrapped 95% confidence intervals (CIs) were derived. The relative Q-TWiST gain of nal-IRI+5-FU/LV vs OS for 5-FU/LV was calculated. Threshold analyses varied uTOX and uTWiST between 0.1 and 1.0. Scenario analysis was conducted using the per protocol (PP) population.

Compared with pts receiving 5-FU/LV (n=119), those receiving nal-IRI+5-FU/LV (117) had significantly more time in TWiST (mean 3.4 vs 2.4 months) and TOX (1.0 vs 0.3 months), but similar time in REL (2.5 vs 2.7 months). In the base case, nal-IRI+5-FU/LV resulted in 1.3 months (95% CI: 0.4-2.1; 5.1 vs 3.9) greater Q-TWiST (range threshold analyses (RTA; 0.9-1.6 months), with relative Q-TWiST gain of 24% (RTA: 17-31%). In the PP population, Q-TWiST was also significantly superior in MM-398+5-FU/LV pts (Q-TWiST gain=1.8 months; 95% CI: 0.7-3.0).

In NAPOLI-1, nal-IRI+5-FU/LV resulted in statistically significantly and clinically important gains in quality-adjusted survival vs 5-FU/LV alone. This confirms the clinical outcome benefit of nal-IRI+5-FU/LV in patients with mPAC.

### Example 12: Effects of Nanoliposomal Irinotecan (Nal-IRI; MM-398)±5-Fluorouracil and Leucavorin (5-FU/LV) on Quality of Life (QoL) in NAPOLI-1: A Phase 3 Study in Patients (Pts) with Metastatic Pancreatic Adenocarcinoma (mPAC) Previously Treated with Gemcitabine-Based Therapy

Here we report QoL results from the NAPOLI-1 trial: A randomized Phase 3 study of nal-IRI plus 5-FU/LV vs 5FU/LV in pts with mPDAC previously treated with gemcitabine-based therapy.

QoL was assessed using the EORTC-QLQ-C30, which includes functional and symptom scales, and a global health and QoL scale. Patients (Pts) completed the questionnaire at treatment start, every 6 weeks (wks), and 30 days post follow-up visit. Pts who provided baseline and ≧1 subsequent assessment were included. Linear transformations were applied to raw scores to produce reported scores in the 0-100 range. Pts were classified as improved (≧10% increase in scale of breadth at a post-baseline time point and remained above baseline for ≧6 wks), worsened (did not meet improvement criteria and died, or had ≧10% decrease from baseline in scale of breadth at a post-baseline time point), or stable (did not meet criteria for improvement or worsening) for each subscale. Pairwise treatment group comparisons on response classification were performed for each subscale and adjusted for multiplicity to control false discovery rate at 0.05 level for the 15 comparisons.

154 pts were included in the analysis; 69% (49/71) in the nal-IRI+5-FU/LV group and 53% (44/83) in the 5-FU/LV group had evaluable data at 12 wks. At baseline, median Global Health Status scores were near the midpoint of the scoring range, median Functional Scale scores were high, and Symptom Scale scores were low, with baseline values similar between groups. The observed median change in score at 12 wks was 0 for both treatment groups for Global Health Status and for the following subscale scores: Role functioning, emotional functioning, cognitive functioning, social functioning, nausea and vomiting, pain, dyspnea, insomnia, appetite loss, constipation, diarrhea, and financial difficulties. For subscale scores where the median change was not 0 (nal-IRI+5-FU/LV: physical functioning and fatigue), the between-group differences were not substantial. There were no significant differences in the proportion of pts classified as improved, worsened, or stable between the treatment groups. Across subscales, adjusted P values for the comparisons were >0.05 (NS).

Conclusion: While limited by pt numbers, in this analysis nal-IRI+5-FU/LV-treated pts tended to maintain baseline QoL over 12 wks. There were no significant differences versus 5-FU/LV-treated pts in QoL response despite the addition of a second cytotoxic agent.

Final Results of NAPOLI-1: A Phase 3 Study of Nal-IRI (MM-398)±5-Fluorouracil and Leucovorin (5-FU/LV) Vs 5-FU/LV in Metastatic Pancreatic Cancer (mPAC) Previously Treated with Gemcitabine-Based Therapy

Background:

nal-IRI, a liposomal formulation of irinotecan (ONIVYDE, MM-398), plus 5-FU/LV is approved in the US for patients (pts) with mPAC previously treated with gemcitabine-based therapy. Primary analysis (data cutoff, Feb. 14, 2014) of the NAPOLI-1 trial (NCT01494506) showed that, after 313 events, nal-IRI+5-FU/LV significantly improved median overall survival (OS) vs 5-FU/LV (6.1 vs 4.2 mo; HR 0.67; 95% CI 0.49-0.92; P=0.012; Wang-Gillam et al, Lancet. 2016). Here we report the final analysis of NAPOLI-1 (data cutoff, Nov. 16, 2015).

Methods:

417 pts were randomly assigned to nal-IRI 70 mg/m² (equivalent to 80 mg/m² irinotecan HCl trihydrate salt)+5-FU/LV 2400/400 mg/m² q2w (n=117), nal-IRI 100 mg/m² (equivalent to 120 mg/m² irinotecan HCl trihydrate salt) q3w (n=151), or 5-FU/LV 2000/200 mg/m² weekly for weeks 1-4 q6w (n=149). Log-rank P values are 2-sided.

Results:

After 382 events, median OS was improved with nal-IRI+5-FU/LV vs 5-FU/LV (6.2 vs 4.2 mo; HR 0.75; 95% CI 0.57-0.99; P=0.038), but not for nal-IRI vs 5-FU/LV (4.9 vs 4.2 mo; HR 1.07; 95% CI 0.84-1.36; P=0.567). Kaplan-Meier estimates of OS for nal-IRI+5-FU/LV and 5-FU/LV, respectively, were 53% and 38% at 6 mo, and 26% and 16% at 12 mo. Median progression-free survival was longer for nal-IRI+5-FU/LV vs 5-FU/LV (3.1 vs 1.5 mo; HR 0.57; 95% CI 0.43-0.76; P<0.001), but not for nal-IRI vs 5-FU/LV (2.7 vs 1.6 mo; HR 0.81; 95% CI 0.63-1.04; P=0.111). Response rates per RECIST v1.1 were higher for nal-IRI+5-FU/LV vs 5-FU/LV (17% vs 1%; P<0.001) and for nal-IRI vs 5-FU/LV (6% vs 1%; P=0.020). Grade≧3 treatment-emergent adverse events in ≧10% of pts in either nal-IRI arm were neutropenia (28%, 15%, and 1% in the nal-IRI+5-FU/LV, nal-IRI, and 5-FU/LV arms, respectively), fatigue (14%, 6%, and 4%), diarrhea (13%, 21%, and 5%), vomiting (12%, 14%, and 4%), anemia (9%, 11%, and 7%), and hypokalemia (3%, 12%, and 2%).

Conclusions:

Final results from NAPOLI-1 continue to show OS benefit for nal-IRI+5-FU/LV vs 5-FU/LV. No new safety concerns were identified. nal-IRI+5-FU/LV provides a new treatment option for pts with mPAC previously treated with gemcitabine-based therapy.

### Example 13: Effects of Nal-IRI (MM-398)±5-Fluorouracil on Quality of Life (QoL) in NAPOLI-1: A Phase 3 Study in Patients with Metastatic Pancreatic Ductal Adenocarcinoma (mPDAC) Previously Treated with Gemcitabine-Based Therapy

Patients with mPDAC frequently experience a significant symptom burden. This in turn negatively impacts their QoL. nal-IRI, a nanoliposomal formulation of irinotecan, was evaluated with or without 5-FU/LV vs 5-FU/LV in a randomized phase 3 study in patients with mPDAC previously treated with gemcitabine-based therapy (NAPOLI-1). Results showed nal-IRI+5-FU/LV significantly improved overall survival compared with 5-FU/LV (6.1 vs 4.2 months; unstratified hazard ratio 0.67; P=0.012) (Wang-Gillam et al., Lancet 2016). QoL was a secondary endpoint of the study.

QoL was assessed using the European Organization for Research and Treatment of Cancer quality-of-life core questionnaire (EORTC-QLQ-C30), which includes functional scales (physical, role, cognitive, emotional, and social); symptom scales (appetite loss, constipation, diarrhea, dyspnea, fatigue, insomnia, nausea and vomiting, and pain); and a global health and quality-of-life scale. Patients were to complete the questionnaire at treatment start, every 6 weeks, and 30 days post-follow-up visit. The population analyzed included all patients who provided baseline and at ≧1 subsequent EORTC-QLQ-C30 assessment. Linear transformations were applied to the raw scores to produce reported scores in the 0-100 range. In the responder analysis, patients were classified as improved (≧10% increase in scale of breadth at a post-baseline time point and remained above baseline for ≧6 weeks), worsened (did not meet improvement criteria and died, or had ≧10% decrease from baseline in scale of breadth at a post-baseline time point), or stable (did not meet criteria for improvement or worsening) for each subscale. Pairwise treatment group comparisons on response classification were performed for each subscale using Cochran-Mantel-Haenszel testing adjusted for multiplicity with a Benjamini-Hochberg correction to control false discovery rate at 0.05 level for the 15 comparisons.

NAPOLI-1 was an international, open-label, randomized, phase 3 trial. Patients were initially randomized to nal-IRI monotherapy (120 mg/m² irinotecan hydrochloride trihydrate salt equivalent to 100 mg/m² irinotecan free base every 3 weeks) or 5-FU/LV (200 mg/m² LV and 2000 mg/m² 5-FU, every week for the first 4 weeks of each 6-week cycle; protocol version 1). Once safety data for the combination treatment became available from a concurrent study in metastatic colorectal cancer, the protocol was amended to include a third arm, nal-IRI+5-FU/LV (80 mg/m² irinotecan hydrochloride trihydrate salt equivalent to 70 mg/m² irinotecan free base every 2 weeks; 400 mg/m² LV and 2400 mg/m² 5-FU every 2 weeks; protocol version 2).

A total of 154 patients (nal-IRI+5-FU/LV, n=71; 5-FU/LV, n=83) comprised the population for this analysis of which 69% (49/71) of patients in the nal-IRI+5-FU group and 53% (44/83) in the 5-FU/LV group had evaluable data at 12 weeks. At baseline, median Global Health Status scores were near the midpoint of the scoring range, median Functional Scale scores were high, and Symptom Scale scores were low, with baseline values similar between groups. The observed median change in score at 12 weeks was 0 for both treatment groups for Global Health Status and for the following subscale scores: role functioning, emotional functioning, cognitive functioning, social functioning, nausea and vomiting, pain, dyspnea, insomnia, appetite loss, constipation, diarrhea, and financial difficulties. For subscale scores for which the median change was not 0 (nal-IRI+5-FU/LV: physical functioning and fatigue), the between-group differences were not substantial. Additionally, there were no significant differences in the proportion of patients classified as improved, worsened, or stable between the treatment groups. Across subscales, adjusted P values for the comparisons were >0.05 (NS).

In NAPOLI-1, evaluable nal-IRI+5-FU/LV-treated patients with data through 12 weeks tended to maintain baseline QoL over 12 weeks, and there were no significant differences versus the 5-FU/LV-treated patients in QoL response despite the addition of a second cytotoxic agent. These results are limited by the small number of patients and variability in QoL subscale scores.

As of the data cutoff of Feb. 14, 2014 (primary analysis) median overall survival (OS) increased significantly with nal-IRI+5-FU/V relative to 5-FU/LV (6.1 vs 4.2 months; unstratified hazard ratio [HR], 0.67 [95% confidence inteval (CI), 0.49-0.92]; P=0.012), but did not differ significantly between nal-IRI monotherapy and 5-FU/LV (4.9 vs 4.2 months; unstratified HR 0.99 [95% CI, 0.77-1.28]; P=0.94).

Median PFS was significantly longer with nal-IRI+5-FU/V compared with 5-FU/LV (3.1 vs 1.5 months; unstratified HR 0.56; 95% CI, 0.41-0.75; P=0.0001).

Median ORR was significantly higher with nal-IRI+5-FU/V compared with 5-FU/LV (16% vs 1%; P<0.0001).

nal-IRI+5-FU/LV exhibited a manageable safety profile; grade ¾ adverse events (AEs) occurring more frequently with nal-IRI+5-FU/LV vs 5-FU/LV included neutropenia (27% vs 1%), fatigue (14% vs 4%), diarrhea (13% vs 4%), and vomiting (11% vs 3%).

71 patients (61% of the ITT population randomized under protocol version 2) in the nal-IRI+5-FU/LV arm and 57 patients (48% of the ITT population randomized under protocol version 2) in the 5-FU/LV arm provided baseline and ≧1 subsequent EORTC assessment (PRO population). Patient demographics and baseline characteristics were similar between the treatment arms.

No substantial differences were identified in the proportion of patients exhibiting improved, stable, or worsening QoL in symptom scale scores between the nal-IRI+5-FU/LV and 5-FU/LV arms.

Baseline global health status and functional scale scores ranged from 58-83 and were similar between the treatment arms. Overall, there were no appreciable changes from baseline in global health status and functional scale scores between the nal-IRI+5-FU/LV and 5-FU/LV arms. The observed median change from baseline to week 6 in physical functioning score was 6.7 points in both arms; which corresponds to “a little” decrease. Baseline symptom scale scores ranged from 0-33 and were similar between the treatment arms. Overall, there were no appreciable changes from baseline in symptom scale scores between the nal-IRI+5-FU/LV and 5-FU/LV arms. The observed median change from baseline to week 6 in fatigue score was approximately 11 points in the nal-IRI+5-FU/LV arm, which corresponds to a “moderate” increase.

nal-IRI+5-FU/LV significantly improves OS in patients with mPDAC previously treated with gemcitabine-based therapy compared with 5-FU/LV. Global health status and functional scale scores were not significantly different between treatment arms at baseline, and showed no appreciable change over 12 weeks. Median symptom scale scores at baseline ranged from 0-33 (low levels of symptomatology), and showed no appreciable change over 12 weeks. nal-IRI+5-FU/LV provides a new treatment option that does not compromise QoL in patients with mPDAC previously treated with gemcitabine-based therapy.

### Example 14: Safety Across Subgroups in NAPOLI-1: A Phase 3 Study of Nal-IRI (MM-398)±5-Fluorouracil and Leucovorin Versus 5-Fluorouracil and Leucovorin in Metastatic Pancreatic Cancer (mPAC) Previously Treated with Gemcitabine-Based Therapy

Nal-IRI+5-FU/LV significantly improved overall survival compared with 5-FU/LV (6.1 vs 4.2 months; P=0.012) and was generally well tolerated (Wang-Gillam et al, Lancet. 2016). The most common grade≧3 treatment-emergent adverse events (TEAEs) in the nal-IRI+5-FU/LV arm were neutropenia, fatigue, diarrhea, and vomiting. Based on NAPOLI-1, the nal-IRI+5-FU/LV regimen received regulatory approval from the US Food and Drug Administration for the treatment of patients with metastatic adenocarcinoma of the pancreas after disease progression following gemcitabine-based therapy. Here, we present results of a prespecified safety analysis by patient subgroup from NAPOLI-1.

TEAEs were graded by NCI CTCAE v4.0 and coded by MedDRA v14.1 for the following prespecified subgroups: sex, age (<65 vs ≧65 years), ethnicity (white vs Asian), UGT1A1*28 status, prior conventional irinotecan therapy (yes vs no), and prior 5-FU therapy (yes vs no). All TEAEs were followed until resolution or patient discontinuation. Analyses were performed on the safety population (ie, those who received≧1 dose of study medication). Results herein are for the nal-IRI+5-FU/LV arm unless otherwise noted.

Overall, the incidence and severity of TEAEs were similar between men (n=67) and women (n=50). Patients aged≧65 years (n=54) generally had a higher incidence of TEAEs than those<65 years (n=63) (eg, stomatitis: 20.4% vs 7.9%; anemia: 46.3% vs 30.2%), although the most common types of TEAEs were similar regardless of age. Overall, Asian (n=33) patients had a higher incidence of grade≧3 TEAEs than white (n=73) patients (87.9% vs 69.9%), primarily because of an increased incidence of neutropenia (24.2% vs 12.3%) and decreased neutrophil counts (33.3% vs 1.4%); febrile neutropenia was reported in 3.0% of Asian patients and 0 white patients. Gastrointestinal disorders also occurred slightly more frequently in Asian patients than white patients (any grade: 100% vs 87.7%), although diarrhea was less frequent and less severe among Asian patients (any grade: 48.5% vs 61.6%; grade≧3: 3.0% vs 19.2%). The UGT1A1 gene encodes an enzyme responsible for glucuronidation of the active metabolite of irinotecan, SN-38. Patients homozygous for the UGT1A1*28 allele (UGT1A1 7/7 genotype) may be at increased risk for neutropenia during irinotecan treatment due to reduced glucuronidation of SN-38. However, in this analysis, there were no differences in incidence, type, and severity of TEAEs between patients homozygous (n=7) for the UGT1A1*28 allele and those who were not (n=110). There were also no notable differences in the incidence or severity of TEAEs between patients with (n=12) and without (n=105) prior conventional irinotecan therapy, or between patients with (n=50) or without (n=67) prior 5-FU therapy.

Overall, the safety profile of nal-IRI+5-FU/LV was generally similar across patient subgroups, apart from an increased risk of grade≧3 neutropenia/reduced neutrophil counts in Asian patients. The results of this prespecified subgroup analysis further support the tolerability profile of nal-IRI+5-FU/LV in patients with mPAC previously treated with gemcitabine-based therapy.

As of the data cutoff of Feb. 14, 2014, median overall survival (OS) increased significantly with nal-IRI+5-FU/LV relative to 5-FU/LV (6.1 vs 4.2 months; unstratified hazard ratio [HR], 0.67 [95% confidence inteval (CI), 0.49-0.92]; P=0.012), but did not differ significantly between nal-IRI monotherapy and 5-FU/LV (4.9 vs 4.2 months; unstratified HR 0.99 [95% CI, 0.77-1.28]; P=0.94).

Adverse events that resulted in a dose reduction occurred in 39 (33%) patients in the nal-IRI+5-FU/LV arm, 46 (31%) patients in the nal-IRI monotherapy arm, and 5 (4%) patients in the 5-FU/LV arm. Adverse events leading to treatment discontinuation occurred in 13 (11%) patients in the nal-IRI+5-FU/LV arm, 17 (12%) patients in the nal-IRI monotherapy arm, and 10 (7%) patients in the 5-FU/LV arm.

NAPOLI-1 was an international, open-label, randomized, phase 3 trial. Patients were initially randomized to nal-IRI monotherapy (120 mg/m² irinotecan hydrochloride trihydrate salt equivalent to 100 mg/m² irinotecan free base every 3 weeks) or 5-FU/LV (200 mg/m² LV and 2000 mg/m² 5-FU, every week for the first 4 weeks of each 6-week cycle). Once safety data for the combination regimen became available from a concurrent study in metastatic colorectal cancer, the protocol was amended to include a third arm, nal-IRI+5-FU/LV (80 mg/m² irinotecan hydrochloride trihydrate salt [equivalent to 70 mg/m² irinotecan free base], 400 mg/m² LV, and 2400 mg/m² 5-FU over 46 hours, every 2 weeks).

The initial nal-IRI dose in the nal-IRI+5-FU/LV arm was 60 mg/m² for patients homozygous for the UGT1A1*28 allele (TA7/TA7 genotype) and could be increased to the standard dose (80 mg/m²) in the absence of drug-related toxic effects.

Randomization was stratified by baseline albumin levels (≧4.0 g/dL vs <4.0 g/dL) KPS (70 and 80 vs ≧90), and ethnicity (white vs east Asian vs all others).

TEAEs were graded by National Cancer Institute Common Terminology Criteria for Adverse Events, version 4.0, and coded by Medical Dictionary for Regulatory Activities, version 14.1. All TEAEs were followed until resolution or patient discontinuation. The safety analysis population included all patients who received≧1 dose of study drug. The presence of the UGT1A1*28 allele was determined by genotype testing, and homozygous patients were identified (A7/TA7 genotype)

The UGT1A1 gene codes an enzyme responsible for glucuronidation of the active metabolite of irinotecan, SN-3. Patients homozygous for the UGT1A1*28 allele may be at increased risk for neutropenia, diarrhea, and other SN-38 exposure related side-effects during irinotecan treatment because of reduced glucuronidation of SN-38.

Data from the nal-IRI+5-FU/LV arm (nal-IRI combination arm) and the 5-FU/LV arm (control arm) are presented herein (data cutoff of Feb. 14, 2014)

Eligibility Criteria:

Key Inclusion Criteria: Adults≧18 years of age; Histologically or cytologically confirmed PDAC; Documented measurable or nonmeasurable distant metastatic disease (as defined by Response Evaluation Criteria in Solid Tumors, version 1.1); Disease progression after prior gemcitabine or gemcitabine-containing therapy in a neoadjuvant, adjuvant (only if distant metastases occurred within 6 months of completing adjuvant therapy), locally advanced, or metastatic setting; KPS score≧70; Adequate hematologic (including absolute neutrophil count>1.5×109 cells/L), hepatic (including normal serum total bilirubin and albumin levels≧30 g/L), and renal function.

Key Exclusion Criteria: Active central nervous system metastasis; Clinically significant gastrointestinal disorder; Severe arterial thromboembolic events<6 months before inclusion; New York Heart Association class III or IV congestive heart failure, ventricular arrhythmias, or uncontrolled blood pressure; Active infection or uncontrolled fever.

Of the 417 patients included in the intention-to-treat population, 398 (95%) received≧1 dose of any study drug (safety analysis population). Patient demographics and baseline characteristics were well balanced between the nal-IRI combination and control arms.

Median duration of exposure to nal-IRI in the nal-IRI combination arm was 8.7 weeks (interquartile range [IQR], 5.4-22.0 weeks); mean dose intensity of nal-IRI over 6 weeks was 167.5 mg/m² (standard deviation [SD], 52.05 mg/m²). Median duration of exposure to 5-FU was 8.7 weeks (IQR, 5.4-22.0 weeks) in the nal-IRI combination arm and 6.0 weeks (IQR, 5.9-12.1 weeks) in the control arm; mean dose intensities of 5-FU over 6 weeks were 5065.0 mg/m² (SD, 1539.1 mg/m²) and 6718.0 mg/m² (SD, 1770.18 mg/m2), respectively.

Incidence of any-grade and grade≧3 TEAEs was similar between patients aged<65 years and those aged≧65 years in each treatment arm. Grade≧3 TEAEs of note (difference of ≧5% between subgroups): In the nal-IRI combination arm, incidence of vomiting (14.3% vs 7.4%) was higher in patients<65 years; incidence of nausea (11.1% vs 4.8%) was higher in patients≧65 years.

Incidence of any-grade TEAEs was similar between white and east Asian patients in each treatment arm, with the exception of diarrhea, which occurred less frequently in east Asians. Incidence of grade≧3 TEAEs in the control arm was similar between white and east Asian patients (56.5% vs 54.5%), whereas the incidence of grade≧3 TEAEs in the nal-IRI combination arm was higher for east Asians compared with whites (87.9% vs 69.9%). Grade≧3 TEAEs of note (difference of ≧5% between subgroups). In the nal-IRI combination arm, incidence of diarrhea (19.2% vs 3.0%), fatigue (19.2% vs 0%), and vomiting (13.7% vs 6.1%) was higher in white patients; incidence of anemia (21.2% vs 5.5%), neutropenia (54.5% vs 17.8%), and white blood cell decrease (21.2% vs 2.7%) was higher in east Asian patients. In the control arm, incidence of abdominal pain (8.2% vs 2.3%) was higher in white patients; incidence of anemia (13.6% vs 3.5%) was higher in east Asian patients.

UGT1A1*28 Allele (TA7/TA7 Genotype): Although the low number of patients with the TA7/TA7 genotype makes comparison difficult the incidence of any-grade and grade≧3 TEAEs appeared to be similar between patients with or without the TA7/TA7 genotype. In the nal-IRI combination arm, 3 of the 7 patients with the TA7/TA7 genotype were able to escalate the nal-IRI dose to 80 mg/m² without needing dose reduction. 1 patient escalated but required dose reduction back to 60 mg/m²; 2 patients maintained the initial dose; 1 patient required dose reduction to 40 mg/m²; 1 additional patient in the nal-IRI combination arm with the TA7/TA7 genotype discontinued treatment (without dose reduction) because of grade 3 vomiting.

Incidence of any-grade and grade≧3 TEAEs was similar between patients with albumin levels≧4.0 g/dL or <4.0 g/dL. Grade≧3 TEAEs of note (difference of ≧5% between subgroups). In the nal-IRI combination arm, incidence of diarrhea (17.6% vs 6.4%) and fatigue (16.2% vs 10.6%) was higher in patients with albumin levels≧4.0 g/dL. In the control arm, incidence of diarrhea (8.1% vs 1.4%) was higher in patients with albumin levels<4.0 g/dL.

Karnofsky Performance Status: Incidence of any-grade TEAEs was similar between patients with KPS score of ≧90 or <90. Incidence of grade≧3 TEAEs was similar between patients with KPS score of ≧90 or <90 in the nal-IRI combination arm; incidence of grade≧3 TEAEs was lower in patients with KPS score of ≧90 vs patients with KPS score of <90 in the control arm (40.9% vs 70.6%). Grade≧3 TEAEs of note (difference of ≧5% between subgroups): In the nal-IRI combination arm, incidence of decreased appetite (8.3% vs 1.4%) and abdominal pain (10.4% vs 4.3%) was higher in patients with KPS score<90; In the control arm, incidence of abdominal pain (8.8% vs 3.0%) was higher in patients with KPS score<90.

The safety profiles for nal-IRI+5-FU/V and 5-FU/LV were generally similar across patient subgroups; diarrhea, vomiting, and nausea were the most commonly occurring TEAEs. The incidence of grade≧3 TEAEs within the subgroups was in line with the safety profile of the overall population. Study limitations included small patient numbers in some of the subgroups and lack of statistical analysis, which preclude definitive conclusions from being drawn. The results of this subgroup analysis further support that nal-IRI+5-FU/LV has a manageable safety profile in patients with mPAC previously treated with gemcitabine-based therapy.

What is claimed is:

1. A method of treating metastatic adenocarcinoma of the pancreas in a human patient who has previously been treated with the antineoplastic agent gemcitabine and has a baseline CA 19-9 level of at least 1,540 U/mL, the method comprising intravenously administering to the patient once every two weeks, an antineoplastic therapy consisting of: 80 mg/m² of the antineoplastic agent MM-398 liposomal irinotecan in combination with 200 mg/m² of (l)-form of leucovorin and 2,400 mg/m² of the antineoplastic agent 5-fluorouracil to treat the metastatic adenocarcinoma of the pancreas in the human patient.

2. The method of claim 1, wherein the patient has a baseline CA 19-9 level of from about 1542 U/mL to about 12,815 U/mL.

3. The method of claim 1, wherein the patient has a baseline CA 19-9 level of 12,815 U/mL or more.

4. The method of claim 1, wherein the 200 mg/m² of (l)-form of leucovorin is administered as 400 mg/m² of the (l+d) racemic form of leucovorin.

5. The method of claim 1, wherein the method comprises at least three cycles administered to the human patient for the metastatic adenocarcinoma of the pancreas.

6. The method of claim 5, wherein the MM-398 liposomal irinotecan, leucovorin and 5-fluorouracil are administered to the patient over 48 hours beginning on day 1 of a 2-week cycle.

7. The method of claim 6, wherein the 200 mg/m² of (l)-form of leucovorin is administered as 400 mg/m² of the (l+d) racemic form of leucovorin, and the leucovorin is administered to the patient prior to the 5-fluorouracil.

8. The method of claim 7, wherein the MM-398 liposomal irinotecan is administered to the patient prior to the leucovorin.

9. The method of claim 8, wherein the MM-398 liposomal irinotecan is administered as a 90 minute infusion, followed by administration of the leucovorin over 30 minutes, followed by administration of the 5-fluorouracil over 46 hours.

10. A method of treating metastatic adenocarcinoma of the pancreas in a human patient who has previously been treated with the antineoplastic agent gemcitabine and the patient has a baseline CA 19-9 level of at least 1542 U/mL, the method comprising intravenously administering to the patient once every two weeks, an antineoplastic therapy consisting of 80 mg/m² of the antineoplastic agent MM-398 liposomal irinotecan in combination with 200 mg/m² of (l)-form of leucovorin or 400 mg/m² of the (l+d) racemic form of leucovorin and 2,400 mg/m² of the antineoplastic agent 5-fluorouracil to treat the metastatic adenocarcinoma of the pancreas in the human patient, wherein the method comprises at least three cycles.

11. The method of claim 10, wherein the patient has a baseline CA 19-9 level of from about 1542 U/mL to about 12,815 U/mL.

12. The method of claim 10, wherein the patient has a baseline CA 19-9 level of at least about 12,815 U/mL.

13. The method of claim 10, wherein the leucovorin is administered as 400 mg/m² of the (l+d) racemic form of leucovorin.

14. The method of claim 10, wherein no other antineoplastic agent is administered to the human patient for treatment of the metastatic adenocarcinoma of the pancreas.

15. The method of claim 14, wherein the MM-398 liposomal irinotecan, leucovorin and 5-fluorouracil are administered to the patient over 48 hours beginning on day 1 of a 2-week cycle.

16. The method of claim 15, wherein 400 mg/m² of the (l+d) racemic form of leucovorin is administered to the patient prior to the 5-fluorouracil.

17. The method of claim 16, wherein the MM-398 liposomal irinotecan is administered to the patient prior to the leucovorin.

18. The method of claim 17, further comprising pre-medicating the patient with dexamethasone and a 5-HT3 antagonist or other anti-emetic prior to administering the MM-398 liposomal irinotecan.

19. A method of treating metastatic adenocarcinoma of the pancreas in a human patient who has previously been treated with the antineoplastic agent gemcitabine and has a baseline CA 19-9 level of less than 120 U/mL, the method comprising intravenously administering to the patient once every two weeks, 80 mg/m² of the antineoplastic agent MM-398 liposomal irinotecan in combination with 200 mg/m² of (l)-form of leucovorin or 400 mg/m² of the (l+d) racemic form of leucovorin and 2,400 mg/m² of the antineoplastic agent 5-fluorouracil to treat the metastatic adenocarcinoma of the pancreas in the human patient.

20. The method of claim 19, wherein the 200 mg/m² of (l)-form of leucovorin is administered as 400 mg/m² of the (l+d) racemic form of leucovorin.
